(19) United States
(12) Patent Application Publication
(10) Pub. No.: US 2023/0303665 A1
(43) Pub. Date: Sep. 28, 2023

(54) MODIFIED ANTI-VIRAL BINDING AGENTS

(71) Applicant: Sana Biotechnology, Inc., Seattle, WA (US)

(72) Inventors: Richard C. MULLIGAN, Cambridge, MA (US); Michael J. VOLLES, Cambridge, MA (US)

(73) Assignee: Sana Biotechnology, Inc., Seattle, WA (US)

(21) Appl. No.: 18/023,331

(22) PCT Filed: Aug. 30, 2021
(86) PCT No.: PCT/US2021/048253

§ 371 Date: Feb. 24, 2023

Related U.S. Application Data
(60) Provisional application No. 63/186,019, filed on May 7, 2021.
(60) Provisional application No. 63/117,864, filed on Nov. 24, 2020.
(60) Provisional application No. 63/072,042, filed on Aug. 28, 2020.

(51) Int. Cl. C07K 16/10; A61P 29/00; A61P 31/14; A61K 39/215

(52) U.S. Cl. CPC C07K 16/10; A61P 29/00; A61P 31/14; A61K 39/215; A61K 2039/505

(57) ABSTRACT

Provided herein are methods of treating a virus infection, such as a coronavirus infection, by delivering to a subject in need a binding agent, wherein the binding agent comprising (i) at least one binding domain that binds to a viral protein expressed on the surface of a virus, and (ii) a modified Fc domain that exhibits either (a) reduced binding to an Fc activating receptor or (b) increased binding to an Fc inhibitory receptor compared to a wild-type Fc domain. The binding agent may be delivered as a polynucleotide (e.g. mRNA) or as a protein, and may be contained in a vehicle for delivery, such as a viral or non-viral vector. The present disclosure also relates to the polynucleotides, proteins, and vehicles (e.g. viral and non-viral vector) and composition thereof, including for use in the methods.

### CROSS-REFERENCE TO RELATED APPLICATIONS

This application claim priority to U.S. provisional application 63/072,042 entitled “Modified Anti-viral Binding Agents”, filed Aug. 28, 2020, to U.S. provisional application 63/117,864 entitled “Modified Anti-viral Binding Agents”, filed Nov. 24, 2020, and to U.S. provisional application 63/186,019 entitled “Modified Anti-viral Binding Agents”, filed May 7, 2021, the contents of each of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 18615_2004040_SEQLISTING created Aug. 27, 2021 which is 277,137 bytes in size. The information in the electronic format of the Sequence Listing is incorporated by reference in its entirety.

### FIELD

The present disclosure relates to methods of treating a virus infection, such as a coronavirus infection, by delivering to a subject in need a binding agent, wherein the binding agent comprising (i) at least one binding domain that binds to a viral protein expressed on the surface of a virus, and (ii) a modified Fc domain that exhibits either (a) reduced binding to an Fc activating receptor or (b) increased binding to an Fc inhibitory receptor compared to a wild-type Fc domain. The binding agent may be delivered as a polynucleotide (e.g. mRNA) or as a protein, and may be contained in a vehicle for delivery, such as a viral or non-viral vector. The present disclosure also relates to the polynucleotides, proteins, and vehicles (e.g. viral and non-viral vector) and composition thereof, including for use in the methods.

### BACKGROUND

Coronaviruses (CoVs) constitute a group of phylogenetically diverse enveloped viruses that encode the largest plus strand RNA genomes and replicate efficiently in most mammals. Human CoV infections typically result in mild to severe upper and lower respiratory tract disease. Severe Acute Respiratory Syndrome Coronavirus (SARS-CoV) emerged in 2002-2003 causing acute respiratory distress syndrome (ARDS) with 10% mortality overall and up to 50% mortality in aged individuals. Middle Eastern Respiratory Syndrome. Coronavirus (MERS-CoV) emerged in the Middle East in April of 2012, manifesting as severe pneumonia, acute respiratory distress syndrome (ARDS) and acute renal failure. More recently, SARS-CoV-2, which is the strain of coronavirus that causes coronavirus disease 2019 (COVID-19), has emerged as an infectious strain in humans.

There are limited therapies for the treatment or prevention of coronavirus infection, including SARS-CoV-2 infection. Many of the drugs under investigation were originally designed for other pathogens and were promptly repurposed for the current COVID-19 trial, including remdesivir, hydroxychloroquine, and favipiravir. In some aspects, there is insufficient evidence that any existing anti-viral drugs can efficiently treat coronavirus infection. There remains a need for improved therapies for treating virus infection, including coronavirus infections, including those associated with SARS-CoV-2.

### SUMMARY

Provided herein are binding agents, polynucleotides, vectors, and methods of delivery of the same for reducing inflammation in a subject in response to a viral infection. Also provided herein are binding agents, polynucleotides, vectors, and methods of delivery of the same for modulating immune complex function, such as inhibiting activating immune complexes and promoting inhibitory immune complex function. In some aspects, the methods further comprise treatment of a viral infection.

Provided herein is a binding agent, comprising: (i) at least one binding domain that binds to a viral protein exposed on the surface of a virus, and (ii) a modified Fc domain, wherein the binding agent is capable of neutralizing the virus and exhibits reduced pro-inflammatory activity compared to an unmodified Fc domain. Also provided herein is a binding agent, comprising: (i) at least one binding domain that binds to a viral protein exposed on the surface of a virus, and (ii) an Fc domain with reduced pro-inflammatory activity as compared to an IgG1 Fc domain, wherein the binding agent is capable of neutralizing the virus.

In some of any of the provided embodiments, the Fc domain is an IgG2 or IgG4 Fc domain. In some of any of the provided embodiments, the modified Fc exhibits reduced binding to an Fc activating receptor. In some of any of the provided embodiments, the modified Fc exhibits increased binding to an Fc inhibitory receptor compared to a wild-type Fc domain.

Also provided herein is binding agent, comprising (i) at least one binding domain that binds to a surface exposed viral protein, and (ii) a modified Fc domain wherein the modified Fc domain has decreased binding to at least one Fc activating receptor family member compared to the wild-type Fc domain. In some of any of the provided embodiments, the Fc activating receptor is Fc gamma receptor I (FcγRI), Fc gamma receptor IIA (FcγRIIA) or Fc gamma receptor III (FcγRIII).

In some of any of the provided embodiments, the binding agent is capable of forming an immune complex with decreased pro-inflammatory activity compared to an immune complex formed with a binding agent comprising the at least one binding domain and a wild-type Fc domain.

In some of any of the provided embodiments, the modified Fc domain comprises an amino acid substitution selected from, Ser228Pro, Glu233Pro, Leu234Ala, Leu234Glu, Leu235Ala, Leu235Glu, Leu235Phe, Gly236Arg, Gly237Ala, Pro238Ser, Asp265Ala, His268Ala, His268Gln, Ser288Pro, Asn297Ala, Asn297Gly, Asn297Gln, Val309Leu, Gly318Ala, Leu328Arg, Pro329Gly, Ala330Ser, and Pro331Ser, each based on EU numbering, or combinations of any of the foregoing. In some of any of the provided embodiments, the modified Fc domain comprises a Leu235Glu substitution based on EU numbering. In some of any of the provided embodiments, the modified Fc domain comprises a Leu234Ala substitution based on EU numbering and Leu235Ala substitution based on EU numbering. In some of any of the provided embodiments, the modified Fc domain comprises a Ser288Pro substitution based on EU numbering and Leu235Glu substitution based on EU numbering. In some of any of the provided embodiments, the modified Fc domain comprises a Leu234Ala substitution based on EU numbering, Leu235Ala substitution based on EU numbering, and Pro329Gly substitution based on EU numbering. In some of any of the provided embodiments, the modified Fc domain comprises a Pro331Ser substitution based on EU numbering, Leu234Glu substitution based on EU numbering, and Leu235Phe substitution based on EU numbering.

In some of any of the provided embodiments, the modified Fc domain comprises a Asp265Ala substitution based on EU numbering. In some of any of the provided embodiments, the modified Fc domain comprises a Gly237Ala substitution based on EU numbering. In some of any of the provided embodiments, the modified Fc domain comprises a Gly318Ala substitution based on EU numbering. In some of any of the provided embodiments, the modified Fe domain comprises a Glu233Pro substitution based on EU numbering. In some of any of the provided embodiments, the modified Fe domain comprises a Gly236Arg substitution based on EU numbering, Leu328Arg substitution based on EU numbering, and Pro329Gly substitution based on EU numbering. In some of any of the provided embodiments, the modified Fe domain comprises a His268Gln substitution based on EU numbering, Val309Leu substitution based on EU numbering, and Ala330Ser substitution based on EU numbering, and/or Pro331Ser substitution based on EU numbering. In some of any of the provided embodiments, the modified Fe domain comprises a Leu234Ala substitution based on EU numbering, Leu235Ala substitution based on EU numbering, Gly237Ala substitution based on EU numbering, Pro238Ser substitution based on EU numbering, His268Ala substitution based on EU numbering, Ala330Ser substitution based on EU numbering, and Pro331Ser substitution based on EU numbering. In some of any of the provided embodiments, the modified Fe domain comprises a Asn297Ala substitution based on EU numbering, Asn297Gly substitution based on EU numbering, or Asn297Gln substitution based on EU numbering. In some of any of the provided embodiments, the modified Fe domain comprises a Ser228Pro substitution based on EU numbering, Phe234Ala substitution based on EU numbering, and Leu235Ala substitution based on EU numbering.

Also provided herein is a binding agent, comprising (i) at least one binding domain that binds to a viral protein exposed on the surface of a virus, and (ii) a modified Fe domain wherein the modified Fe domain has increased binding to an inhibitory Fc receptor compared to the wild-type Fe domain.

In some of any of the provided embodiments, the inhibitory Fc receptor is an FcγRIIB, optionally wherein the FcRIIB is FcγRIIB1 or FcγRIIB2. In some of any of the provided embodiments, the binding agent is capable of forming an immune complex with increased anti-inflammatory activity compared to an immune complex formed with a binding agent comprising the at least one binding domain and a wild-type Fe domain. In some of any of the provided embodiments, the binding agent is capable of forming an immune complex with decreased inflammatory activity compared to an immune complex formed with a binding agent comprising the at least one binding domain and a wild-type Fe domain. In some of any of the provided embodiments, the modified Fe domain comprises an amino acid substitution selected from, Phe241Ala, Ser267Glu, His268Phe, Leu328Phe, Ser324Thr, Pro238Asp, Leu328Glu, Ser239Asp, Ile332Glu, Gly236Ala each based on EU numbering, or combinations of any of the foregoing.

In some of any of the provided embodiments, the modified Fc domain comprises a Ser267Glu substitution based on EU numbering and His268Phe substitution based on EU numbering, and Ser324Thr substitution based on EU numbering. In some of any of the provided embodiments, the modified Fc domain comprises a Ser267Glu substitution based on EU numbering and Leu328Phe substitution based on EU numbering. In some of any of the provided embodiments, the modified Fc domain comprises a Pro238Asp substitution based on EU numbering. In some of any of the provided embodiments, the modified Fc domain comprises a Leu328Glu substitution based on EU numbering.

In some of any of the provided embodiments, the modified Fc domain comprises a Ser239Asp substitution based on EU numbering and Ile332Glu substitution based on EU numbering. In some of any of the provided embodiments, the modified Fc domain comprises a Ser239Asp substitution based on EU numbering and Ile332Glu substitution based on EU numbering, and Gly236Ala substitution based on EU numbering. In some of any of the provided embodiments, the modified Fc domain comprises a Ser267Glu substitution based on EU numbering. In some of any of the provided embodiments, the modified Fc domain comprises a E233D substitution based on EU numbering. In some of any of the provided embodiments, the modified Fc domain comprises a G237D substitution based on EU numbering. In some of any of the provided embodiments, the modified Fc domain comprises a H268D substitution based on EU numbering.

In some of any of the provided embodiments, the modified Fc domain comprises a P271G substitution based on EU numbering. In some of any of the provided embodiments, the modified Fc domain comprises a A330R substitution based on EU numbering. In some of any of the provided embodiments, the modified Fc domain comprises a E233D substitution based on EU numbering and a A330R substitution based on EU numbering. In some of any of the provided embodiments, the modified Fc domain comprises a E233D substitution based on EU numbering, a P271G substitution based on EU numbering, and a A330R substitution based on EU numbering. In some of any of the provided embodiments, the modified Fc domain comprises a G237D substitution based on EU numbering, a H268D substitution based on EU numbering, and a P271G substitution based on EU numbering. In some of any of the provided embodiments, the modified Fc domain comprises a G237D substitution based on EU numbering, a P271G substitution based on EU numbering, and a A330R substitution based on EU numbering. In some of any of the provided embodiments, the modified Fe domain comprises a E233D substitution based on EU numbering, a H268D substitution based on EU numbering, a P271G substitution based on EU numbering, and a A330R substitution based on EU numbering. In some of any of the provided embodiments, the modified Fe domain comprises a G237D substitution based on EU numbering, a H268D substitution based on EU numbering, a P271G substitution based on EU numbering, and a A330R substitution based on EU numbering. In some of any of the provided embodiments, the modified Fe domain comprises a E233D substitution based on EU numbering, a G237D substitution based on EU numbering, a H268D substitution based on EU numbering, a P271G substitution based on EU numbering, and a A330R substitution based on EU numbering.

In some of any of the provided embodiments, the modified Fe domain comprises one or more amino acid substitutions compared to wildtype Fe domain. In some of any of the provided embodiments, the wildtype Fe domain is a wildtype IgG1. In some of any of the provided embodiments, the wildtype Fe domain comprises the sequence of amino acids set forth in SEQ ID NO 1. In some of any of the provided embodiments, the modified Fe domain comprises a sequence of amino acids that exhibits at least 85%, at least 90%, at least 92%, at least 95%, at least 97% sequence identity to SEQ ID NO 1.

In some of any of the provided embodiments, the wildtype Fe domain is a wildtype IgG2. In some of any of the provided embodiments, the wildtype Fe domain comprises the sequence of amino acids set forth in SEQ ID NO 2. In some of any of the provided embodiments, the modified Fe domain comprises a sequence of amino acids that exhibits at least 85%, at least 90%, at least 92%, at least 95%, at least 97% sequence identity to SEQ ID NO 2.

In some of any of the provided embodiments, the wildtype Fe domain is a wildtype IgG4. In some of any of the provided embodiments, the wildtype Fe domain comprises the sequence of amino acids set forth in SEQ ID NO 3. In some of any of the provided embodiments, the modified Fe domain comprises a sequence of amino acids that exhibits at least 85%, at least 90%, at least 92%, at least 95%, at least 97% sequence identity to SEQ ID NO 3. In some of any of the provided embodiments, the modified Fe domain comprises a sequence of amino acids that exhibits at least 85% sequence identity to any of SEQ ID NO 1-3.

In some of any of the provided embodiments, the at least one binding domain and modified Fe domain are directly linked. In some of any of the provided embodiments, the at least one binding domain and modified Fe domain are indirectly linked via a linker. In some of any of the provided embodiments, the linker is a peptide linker. In some of any of the provided embodiments, the peptide linker is (GmS)n (SEQ ID NO: 4), wherein each of m and n is an integer between 1 to 4, inclusive.

In some of any of the provided embodiments, the at least one binding domain is at least two binding domains. In some of any of the provided embodiments, the at least two binding domains bind at least two distinct epitopes of the viral protein. In some of any of the provided embodiments, the at least two binding domains are directly linked. In some of any of the provided embodiments, the at least two binding domains are indirectly linked via a linker. In some of any of the provided embodiments, the linker is a peptide linker. In some of any of the provided embodiments, the peptide linker is (GmS)n (SEQ ID NO: 4), wherein each of m and n is an integer between 1 to 4, inclusive.

In some of any of the provided embodiments, the viral protein is a viral receptor. In some of any of the provided embodiments, the virus is an RNA virus. In some of any of the provided embodiments, the virus is an orthomyxovirus, optionally wherein the virus is an influenza virus. In some of any of the provided embodiments, the virus is a paramyxovirus. In some of any of the provided embodiments, the virus is Respiratory Syncytial Virus (RSV). In some of any of the provided embodiments, the virus is Measles morbillivirus (MeV). In some of any of the provided embodiments, the virus is a coronavirus. In some of any of the provided embodiments, the virus is Severe Acute Respiratory Syndrome (SARS) CoV-2. In some of any of the provided embodiments, the virus is a variant of SARS CoV-2. In some of any of the provided embodiments, the virus is a SARS CoV-2 Variant of Interest (Vol), Variant of Concern (VoC), and/or Variant of High Consequence (VoHC). In some of any of the provided embodiments, the SARS CoV-2 variant is chosen from the group comprising: Alpha (i.e., B.1.1.7), Beta (i.e., B.1.351, B.1.351.2, B.1.351.3), Delta (i.e., B.1.617.2, AY.1, AY.2, AY.3), and Gamma (i.e., P.1, P.1.1, P.1.2). In some of any of the provided embodiments, the virus is SARS CoV-1. In some of any of the provided embodiments, the virus is Middle Eastern Respiratory Syndrome Virus (MERS-V).

In some of any of the provided embodiments, the binding agent is a dimer. In some of any of the provided embodiments, the binding agent is capable of neutralizing the virus. In some of any of the provided embodiments, the binding agent reduces or prevents viral attachment to a host cell. In some of any of the provided embodiments, the at least one binding domain comprises an antigen-binding fragment of an antibody that specifically binds the viral protein.

In some of any of the provided embodiments, the at least one binding domain specifically binds the S (spike) glycoprotein of a SARS virus.

In some of any of the provided embodiments, the antigen-binding fragment comprises a variable heavy chain (VH) and a variable light chain (VL). In some of any of the provided embodiments, the antigen-binding fragment is selected from among a Fab fragment, F(ab′)2 fragment, Fab′ fragment, Fv fragment. In some of any of the provided embodiments, the at least one binding domain specifically binds the S (spike) glycoprotein of a SARS virus. In some of any of the provided embodiments, the at least one binding domain is an antigen-binding fragment of an antibody selected from among STI-1499, STI-4398, REGN10933, REGN10987, REGN-COV2 and JS016. In some of any of the provided embodiments, the at least one binding domain is an antigen-binding fragment of an antibody selected from among STI-1499, STI-4398, STI-2020 REGN10933, REGN10987, REGN-COV2, JS016, LY-CoV555, LY-3819253, TB181-8, TB181-28, TB181-36, BGB-DXP593, TY027, CT-P59, BRII-196, BRII-198, SCTA01, MW33, AZD8895, AZD1061, HLX70, 15G11, 18F4, 1E5, 1G3, 21C3, 22d(23D11, 26E2, 29F7, 3B3, 3F2, D59047-11955, D70678-12637-S1, D70678-12799-S1, D70678-13531-S1, D70678-13576-S1, D70678-14004-S2, D70678-14027-S2, D70678-2155-S1, D70678-2743-S1, or D70678-5521-S2.

In some of any of the provided embodiments, the at least one binding domain is a single domain antibody (sdAb). In some of any of the provided embodiments, the at least one binding domain is a single domain antibody selected from among TB201-1, TB202-3, TB202-63,

In some of any of the provided embodiments, the at least one binding domain is an antibody mimetic, optionally selected from Designed Ankyrin Repeat Protein (DARPin), adnectins, or an antigen-binding fibronectin type III (Fn3) scaffold.

Provided herein is a particle, comprising (i) the binding agent of any of claims 1-88 and (ii) a viral protein capable of being bound by the at least one binding domain of the binding agent.

In some of any of the provided embodiments, the viral protein is a purified viral protein. In some of any of the provided embodiments, the viral protein is a recombinant viral protein. In some of any of the provided embodiments, the viral protein is the S (spike) glycoprotein of a SARS virus. In some of any of the provided embodiments, the viral protein is the S glycoprotein of a SARS coronavirus 1 (SARS CoV-1). In some of any of the provided embodiments, the viral protein is the S glycoprotein of a SARS coronavirus 2 (SARS CoV-2). In some of any of the provided embodiments, the viral protein is the S glycoprotein of a SARS CoV-2 variant. In some of any of the provided embodiments, the viral protein is the S glycoprotein of a SARS CoV-2 Variant of Interest (Vol), Variant of Concern (VoC), and/or Variant of High Consequence (VoHC). In some of any of the provided embodiments, the viral protein is the S glycoprotein of a SARS CoV-2 variant chosen from the group comprising: Alpha (i.e., B.1.1.7), Beta (i.e., B.1.351, B.1.351.2, B.1.351.3), Delta (i.e., B.1.617.2, AY.1, AY.2, AY.3), and Gamma (i.e., P.1, P.1.1, P.1.2).

Provided herein is a nucleic acid molecule, encoding any of the binding agents provided herein. In some of any of the provided embodiments, the at least one binding domain of the binding agent comprises a variable heavy chain (VH) and a variable light chain (VL), wherein the nucleic acid comprises a first sequence encoding the VH chain and the modified Fc and a second sequence encoding the VL chain and wherein the first and second sequence are separated by a multicistronic element. In some of any of the provided embodiments, the multicistronic element(s) comprises a sequence encoding a ribosome skip element selected from among a T2A, a P2A, a E2A or a F2A or an internal ribosome entry site (IRES). In some of any of the provided embodiments, the first sequence and the second sequence are operably connected to the same promoter. In some of any of the provided embodiments, the nucleic acid molecule is mRNA.

Provided herein is a cell, such as a cell comprising any of the provided nucleic acids.

Also provided herein is a vector, such as a vector comprising any of the nucleic acid molecules provided herein. In some of any of the provided embodiments, the vector is a viral vector or viral like particle. In some of any of the provided embodiments, the vector is derived from an adenovirus. In some of any of the provided embodiments, the viral vector is derived from an Adeno-associated virus (AAV). In some of any of the provided embodiments, the AAV is of serotype 1, 2, 5, 6, 8 or 9. In some of any of the provided embodiments, the AAV is of serotype 8. In some of any of the provided embodiments, the AAV is of serotype 6. In some of any of the provided embodiments, the AAV is of serotype 6.2. In some of any of the provided embodiments, the viral vector is derived from a lentivirus. In some of any of the provided embodiments, the lentivirus is Human Immunodeficiency Virus-1 (HIV-1).

In some of any of the provided embodiments, the viral vector or viral-like particle comprises a fusogen. In some of any of the provided embodiments, the vector is a lipid particle, wherein the lipid particle comprises (i) a lipid bilayer enclosing a lumen, and (ii) a fusogen, wherein the fusogen is embedded in the lipid bilayer. In some of any of the provided embodiments, the lipid bilayer is derived from a membrane of a host cell used for producing a virus or virus-like particle. In some of any of the provided embodiments, the lipid bilayer is derived from a membrane of a host cell used for producing a virus-like particle, wherein the virus-like particle is replication defective. In some of any of the provided embodiments, the fusogen is a viral fusogen selected from a Class I viral membrane fusion protein, a Class II viral membrane protein, a Class II viral membrane fusion protein, a viral membrane glycoprotein, or a viral envelope protein.

In some of any of the provided embodiments, the fusogen is a vesicular stomatitis virus envelope glycoprotein (VSV-G). In some of any of the provided embodiments, the fusogen is a baboon endogenous virus (BaEV) envelope glycoprotein. In some of any of the provided embodiments, the fusogen is a syncytin. In some of any of the provided embodiments, the fusogen is from a coronavirus. In some of any of the provided embodiments, the fusogen is a Severe Acute Respiratory Syndrome (SARS) coronavirus 1 (SARS CoV-1) spike glycoprotein. In some of any of the provided embodiments, the fusogen is a Severe Acute Respiratory Syndrome (SARS) coronavirus 2 (SARS CoV-2) spike glycoprotein. In some of any of the provided embodiments, the fusogen is an alpha coronavirus CD13 protein.

In some of any of the provided embodiments, the fusogen comprises an F protein molecule or a biologically active portion thereof from a Paramyxovirus and/or a glycoprotein G (G protein) or a biologically active portion thereof from a Paramyxovirus. In some of any of the provided embodiments, the fusogen is derived from an F protein molecule or a biologically active portion thereof from a Paramyxovirus and/or a glycoprotein G (G protein) or a biologically active portion thereof from a Paramyxovirus. In some of any of the provided embodiments, the Paramyxovirus is a henipavirus. In some of any of the provided embodiments, the Paramyxovirus is Nipah virus. In some of any of the provided embodiments, the Paramyxovirus is Hendra virus.

In some of any of the provided embodiments, the fusogen is a re-targeted fusogen comprising a targeting moiety that binds to a molecule on a target cell. In some of any of the provided embodiments, the targeting moiety is a Design ankyrin repeat proteins (DARPin), a single domain antibody (sdAb), a single chain variable fragment (scFv), or an antigen-binding fibronectin type III (Fn3) scaffold.

In some of any of the provided embodiments, the target cell is known or suspected of being infected by a coronavirus. In some of any of the provided embodiments, the targeting moiety binds a receptor of a coronavirus. In some of any of the provided embodiments, the targeting moiety binds angiotensin-converting enzyme 2 (ACE2). In some of any of the provided embodiments, the target cell is a B lymphocyte. In some of any of the provided embodiments, the targeting moiety binds to human CD20.

In some of any of the provided embodiments, the fusogen is modified to reduce its native binding tropism. In some of any of the provided embodiments, the G protein or the biologically active portion thereof is a mutant NiV-G protein that exhibits reduced binding to Ephrin B2 or Ephrin B3.

In some of any of the provided embodiments, the mutant NiV-G protein comprises one or more amino acid substitutions corresponding to amino acid substitutions selected from the group consisting of E501A, W504A, Q530A and E533A with reference to numbering set forth in SEQ ID NO:5. In some of any of the provided embodiments, the mutant NiV-G protein has the amino acid sequence set forth in SEQ ID NO: 34 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:34. In some of any of the provided embodiments, the mutant NiV-G protein has the amino acid sequence set forth in SEQ ID NO: 35 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35. In some of any of the provided embodiments, the NiV-F protein is a biologically active portion thereof that has a 20 amino acid truncation at or near the C-terminus of the wild-type NiV-F protein (SEQ ID NO:19). In some of any of the provided embodiments, the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32. In some of any of the provided embodiments, the NiV-F protein is a biologically active portion thereof that has a 22 amino acid truncation at or near the C-terminus of the wild-type NiV-F protein (SEQ ID NO:37). In some of any of the provided embodiments, the NiV-F protein has an amino acid sequence set forth in SEQ ID NO:36 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36. In some of any of the provided embodiments, the NiV-F protein has an amino acid sequence set forth in SEQ ID NO:38 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 38.

In some of any of the provided embodiments, the NiV-F protein comprises a point mutation on an N-linked glycosylation site. In some of any of the provided embodiments, the NiV-F protein is a biologically active portion thereof that comprises: i) a 20 amino acid truncation at or near the C-terminus of the wild-type NiV-F protein (SEQ ID NO:36); and ii) a point mutation on an N-linked glycosylation site.

Provided herein is a method of producing a binding agent, the method comprising introducing the nucleic acid molecule of any of claims 93-98 or vector of any of claims 99-144 into a host cell under conditions to express the binding agent in the cell. In some of any of the provided embodiments, the method further comprises isolating or purifying the binding agent from the cell.

Also provided here is a method of forming an immune complex, the method comprising administering the one or more binding agent of any of the provided to a subject known or suspected of having a viral infection, wherein an immune complex comprising the administered one or more binding agent is formed in the subject.

In some of any of the provided embodiments, the immune complex further comprises at least one endogenous antibody against a viral protein exposed on the surface of the virus. In some of any of the provided embodiments, the one or more binding agent and the at least one endogenous antibody bind the same viral protein. In some of any of the provided embodiments, the one or more binding agent and the at least one endogenous agent bind the same epitope of the viral protein. In some of any of the provided embodiments, the one or more binding agent and the at least one endogenous agent bind a distinct epitope of the viral protein. In some of any of the provided embodiments, the one or more binding agent and the at least one endogenous agent bind an overlapping epitope of the viral protein.

Provided herein is a composition comprising an immune complex comprising: (i) any of the provided binding agents and (ii) a surface exposed viral protein bound by the at least one binding domain of the binding agent.

In some of any of the provided embodiments, the immune complex comprises two or more binding agents. In some of any of the provided embodiments, the at least two binding domains bind a distinct epitope of the viral protein. In some of any of the provided embodiments, the immune complex further comprises endogenous binding domains, optionally from an endogenous antibody and/or antibodies. In some of any of the provided embodiments, the at least two binding domains and endogenous binding domains bind the same epitope of the viral protein. In some of any of the provided embodiments, the at least two binding domains and endogenous binding domains bind a distinct epitope of the viral protein. In some of any of the provided embodiments, the at least two binding domains and endogenous binding domains bind an overlapping epitope of the viral protein.

Provided herein is a pharmaceutical composition, comprising any of the provided binding agents, any of the provided nucleic acids, the provided cells, the provided vectors and/or any of the provided particles.

Also provided herein is a pharmaceutical composition, comprising (i) the binding agent of any of claims 1-88 and (ii) a recombinant viral protein capable of being bound by the at least one binding domain of the binding agent.

In some of any of the provided embodiments, the pharmaceutical composition comprises a pharmaceutically acceptable excipient. In some of any of the provided embodiments, the pharmaceutical composition is sterile.

Provided herein is a method of reducing inflammation in response to a viral infection in a subject, the method comprising administering, to a subject known or suspected of having a virus infection, a therapeutically effective amount of any of the provided binding agents, any of the provided particles, any of the provided the nucleic acids, any of the provided cells, or any of the provided vectors.

Provided herein is a method of reducing inflammation in response to a viral infection in a subject, the method comprising administering to a subject known or suspected of having a virus infection a therapeutically effective amount of any of the provided pharmaceutical compositions.

Also provided herein is a method of reducing inflammation in response to a viral infection in a subject, the method comprising administering to a subject known or suspected of having a virus infection, (i) a therapeutically effective amount of any of the provided pharmaceutical compositions, (ii) a recombinant viral protein capable of being bound by the at least one binding domain of the binding agent.

In some of any of the provided embodiments, the inflammation comprises lymphocytic accumulation in the lung, lymphocytic proliferation in the lung, peripheral blood lymphopenia, pro-inflammatory cytokine production, or combinations of any of the foregoing. In some of any of the provided embodiments, the pro-inflammatory cytokine is selected from the group consisting of: MCP-1, IL-8, IL-1β, IFN-γ, IP-10, IL-4, IL-1β, IL-2, IL-7, GCSF, MIP-1A, and TNF-α.

In some of any of the provided embodiments, the pharmaceutical composition and recombinant viral protein are administered concurrently. In some of any of the provided embodiments, the pharmaceutical composition and recombinant viral protein are administered sequentially, optionally wherein the recombinant viral protein is first administered. In some of any of the provided embodiments, the pharmaceutical composition and recombinant viral protein are administered sequentially, optionally wherein the pharmaceutical composition is first administered.

Provided herein is a method of promoting inhibitory immune complex function, comprising administering therapeutically effective amount of any of the provided pharmaceutical composition.

Also provided herein is a method of promoting inhibitory immune complex function, comprising administering therapeutically effective amount of any of the provided binding agents, any of the provided particles, any of the provided nucleic acids, any of the provided cells, any of the provided vectors.

In some of any of the provided embodiments, the inhibitory immune complex function comprises diminished antigen uptake, diminished antigen presentation, reduced cellular activation, reduced antibody secretion, production of anti-inflammatory cytokines, or combinations of any of the foregoing.

Provided herein is a method of reducing activating immune complex function, comprising administering therapeutically effective amount of any of the provided pharmaceutical compositions.

Also provided herein is a method of reducing activating immune complex function, comprising administering therapeutically effective amount of any of the provided binding agents, any of the provided particles, any of the provided nucleic acids, any of the provided cells, any of the provided vectors.

In some of any of the provided embodiments, the activating immune complex function comprises antibody-dependent cell mediated cytotoxicity (ADCC), antibody dependent enhancement (ADE), release of inflammatory mediators, production of pro-cytokines, phagocytosis, or combinations of any of the foregoing.

In some of any of the provided embodiments, the method further comprises treatment of a viral infection in a subject. In some of any of the provided embodiments, the viral infection is an infection caused by a virus with a surface exposed viral protein recognized by the at least one binding domain of the binding agent. In some of any of the provided embodiments, the virus is an RNA virus. In some of any of the provided embodiments, the virus selected from SARS-CoV-1, SARS-CoV-2, MERS, RSV, influenza viruses, and measles virus.

Also provided herein any of the provided binding agents, particles, nucleic acids, cells, vectors, or pharmaceutical compositions provided herein for use in a method of reducing inflammation in response to a viral infection in a subject. Also provided herein is use of any of the provided binding agents, particles, nucleic acids, cells, vectors, or pharmaceutical compositions provided herein for manufacture of a medicament for reducing inflammation in response to a viral infection in a subject. In some of any of the above embodiments, the method of reducing inflammation in response to a viral infection in a subject further comprises use of a recombinant viral protein capable of being bound by the at least one binding domain of the binding agent.

Provided herein is any of the provided binding agents, particles, nucleic acids, cells, vectors, or pharmaceutical compositions provided herein for use in a method of promoting inhibitor immune complex function. Also provided herein is use of any of the provided binding agents, particles, nucleic acids, cells, vectors, or pharmaceutical compositions provided herein in the manufacture of a medicament for reducing activating immune complex function.

### DETAILED DESCRIPTION

Provided herein are binding agents that contain at least one binding domain that binds to a viral surface protein and neutralizes the virus, wherein the binding agent does not exhibit pro-inflammatory activity. The at least one binding domain can include at least one antibody or antigen-binding fragment that binds to a viral surface protein and neutralizes the virus. In some embodiments, the binding agent does not contain an Fc domain that exhibits effector activity via binding to an activating Fc receptor (FcR). In some embodiments, the binding agent contains the at least one binding domain, e.g. antigen-binding fragment of an antibody, and an Fc domain with reduced pro-inflammatory activity compared to an IgG1 Fc domain. In some embodiments, the binding agent contains the at least one binding domain, e.g. antigen-binding fragment of an antibody, and a modified Fc domain that exhibits reduced pro-inflammatory activity compared to an unmodified Fc domain, such as compared to a wild-type IgG1 Fc domain. Also provided are compositions, methods and uses of the binding agents as therapeutic agents for the treatment of viral infections.

In some embodiments, the binding agent is an antibody mimetic, or is full-length antibody containing an antigen-binding domain (e.g. containing a variable heavy (VH) chain and a variable light (VL)) directed against the viral surface protein, in which the Fc portion of the antibody is a wild-type IgG2 or wild-type IgG4. In some cases, the binding agent is based on a reference antibody containing the antigen-binding domain (e.g. containing a variable heavy (VH) chain and a variable light (VL)) of the reference antibody, but in which an IgG1 Fc of the reference antibody is replaced by an Fc of wildtype IgG2 or wildtype IgG4. In some embodiments, the binding agent is a full-length antibody containing an antigen-binding domain (e.g. containing a variable heavy (VH) chain and a variable light (VL)) directed against the viral surface protein, in which the Fc portion of the antibody is modified compared to the Fc of a reference antibody. In some embodiments, the Fc domain is modified by one or more amino acid substitutions to reduce binding to an Fc activating receptor. In some embodiments, the Fc domain is modified by one or more amino acid substitutions to increase binding to an Fc inhibitory receptor.

In some embodiments, the binding agent contains (i) the at least one binding domain, e.g. antigen-binding fragment of an antibody (e.g. containing a variable heavy (VH) chain and a variable light (VL)), and (ii) an Fc domain that exhibits reduced binding to an Fc activating receptor compared to a wild-type Fc domain, such as a wild-type IgG1 Fc domain. In some embodiments, the Fc domain is a modified Fc domain containing one or more amino acid substitutions to reduce binding to an Fc activating receptor. In some embodiments, the provided binding agents reduce pro-inflammatory immune complex formation or effector function.

In some embodiments, the binding agent contains (i) the at least one binding domain, e.g. antigen-binding fragment of an antibody (e.g. containing a variable heavy (VH) chain and a variable light (VL)), and (ii) an Fc domain that exhibits increased binding to an Fc inhibitory receptor compared to a wild-type Fc domain, such as a wild-type IgG1 Fc domain. In some embodiments, the Fc domain is a modified Fc domain containing one or more amino acid substitutions to increase binding to an Fc inhibitor receptor. In some embodiments, the provided binding agents promote the formation of anti-inflammatory inhibitory immune complexes.

Also provided herein are particles containing any of the provided binding agents and a viral protein capable of being bound by the at least one binding domain of the binding agent (hereinafter also called a particle).

Provided herein are methods of treating a viral infection, such as a coronavirus infection, the method comprising administering any of the provided binding agents to a subject known or suspected of having a viral infection, such as a coronavirus infection. Also provided herein are methods of treating a viral infection, such as a coronavirus infection, the method comprising administering any of the provided particles agent to a subject known or suspected of having a viral infection, such as a coronavirus infection. In some embodiments, the binding agent is administered as a polynucleotide. In some embodiments, the binding agent is administered as a protein. In some embodiments, the protein that is administered as a protein complex where the binding agent is provided as a particle with a viral protein. The polynucleotide or protein also may be contained in a vehicle, such as a viral or non-viral vector, for delivery. In particular embodiments, the provided methods are for use in treating a virus infection. For example, the provided embodiments include methods and uses for treating a Coronavirus infection, such as caused or due to SARS-CoV-2.

In some aspects, pathology of viral diseases, such as COVID-19 caused by infection with SARS CoV-2, is mediated by inflammatory responses. Early onset of viral replication in epithelial and endothelial cells can results in cellular damage and apoptosis. Infection may also result in pyroptosis of certain lymphocytes, including macrophages. In some aspects, the release of pro-inflammatory cytokines coupled with vascular leakage results in pathological inflammation.

In some aspects, sequelae of viral diseases, such as COVID-19 caused by infection with SARS CoV-2, are mediated by inflammatory responses. Anti-viral neutralizing antibodies can facilitate severe lung injury seen following the acute phase of infection with some viruses. For example, anti-SARS CoV-2 Spike glycoprotein antibodies can promote pulmonary inflammation via the accumulation of lymphocytes in the lung, including macrophages which in turn can incite local MCP-1 and IL-8 pro-inflammatory cytokine production (Liu et al., JCI Insight. 4(4): 2019)

The inflammatory response initiated by anti-viral neutralizing antibodies is in some aspects mediated through the binding of the antigen-antibody complex to Fc family receptors (FcR) present on lymphocytes (E.g., monocytes, macrophages, B cells, etc.). Anti-viral neutralizing antibodies may also contribute to viral disease pathology through the fixation of complement components or alternatively via antibody-dependent cell-mediated cytotoxicity (ADCC) or antibody-dependent enhancement (ADE). ADE promotes the uptake and presentation of antibody-antigen complexes thus propagating the immune response, and is also mediated via interaction with FcR (Fu et al., Viro Sin (35):266-271, 2020).

An immune complex (IC) is formed via the binding of multiple antigens to antibodies. The ratio of antigen to antibody can determine the IC shape and overall size. In some aspects, owing to a low affinity for a singular antibody, ICs are more efficient at binding and signaling via FcRs. Exposure to a virus or use of antibody therapeutics that bind to the virus or components of the virus, such as IVIG or monoclonal antibodies, can lead to the generation of IC. ICs in turn can deliver signals to immune cells or to the infected cells themselves via specific types of Fc receptors on those cells. Depending upon the properties of the specific Fc of the antibody, a variety of both productive and non-productive immune reactions may occur. In particular, ICs can lead to immune effector functions such as complement fixation that can kill infected cells, and/or induce the expression of pathological pro-inflammatory molecule from cells which come in contact with the ICs (Monsalvo et al., Nat Med17(2):195-199, 2011)

Accordingly, treatment of patients will likely benefit from the ability to specifically control the outcome of interactions between ICs and immune cells and/or targets of a viral infection. There remains a need for improved therapies for treating virus infection, including coronavirus infections, including those associated with SARS-CoV-2

In certain embodiments, the binding agent or particle is capable of effecting decreased immunological complex formation with the Fc activating receptor compared to the wild-type Fc domain. In certain embodiments, the binding agent or particle is capable of effecting increased immunological complex formation with the Fc inhibitory receptor compared to the wild-type Fc domain. It is contemplated that the provided binding agents therefore can modulate immune complex formation to reduce or avoid pro-inflammatory activity upon binding of the binding domain, e.g. an antibody or antigen-binding fragment, to the viral surface protein.

Fcγ receptors are members of the immunoglobulin gene superfamily of proteins that can bind to the Fcγ portion of an antibody molecule. Members of this family recognize one or more isotypes of antibodies via the recognition domain on the a chain of the Fcγ receptor. Fcγ receptors are defined by their specificity for immunoglobulin subtypes.

Members of the Fcγ receptor family are integral membrane glycoproteins with an extracellular domain associated with the C2 set of immunoglobulin-related domains, a single transmembrane domain, and a variable-length cytoplasmic domain. There are three known FcγRs, FcγRI (also known as CD64), FcγRII (CD32), and FcγRIII (CD16). While the three receptors are encoded by different genes, it is thought their sequence homology is evidence for a gene duplication event.

Various lymphocytic cells can have multiple Fcγ receptors to bind antibodies of different isotypes, and the antibody isotype in some aspects determines which lymphocytes are involved in a given response (Ravetch et al. Science, 290: 84-89, 2000; Ravetch et al., Annu. Rev. Immunol. 19: 275-90, 2001). Table 1A summarizes the various Fc receptors, the cells that express them, and their isotype specificity.

In some aspects, the Fc activating receptor is Fc gamma receptor I (FcγRI), Fc gamma receptor IIA (FcγRIIA) or Fc gamma receptor III (FcγRIII). Both FcγR-mediated activation and repression effector functions are transmitted via FcγR after ligation. Two distinct domains within the cytoplasmic signaling domain of the receptor, the immunoreceptor tyrosine activation motif (ITAM) or the immunoreceptor tyrosine suppression motif (ITIM), allow for opposed immunological responses via the recruitment of different cytoplasmic enzymes to these structures. ITAM-containing FcγR complexes include FcγRI, FcγRIIA, and FcγRIIIA is included, but the ITIM-containing complex includes only FcγRIIB.

For example, neutrophils express the FcγRIIA activating receptor. Fc via immune complex or specific antibody cross-linking FcγRIIA clustering acts to aggregate local receptor-related kinases that facilitate ITAM phosphorylation. ITAM phosphorylation serves as a docking site for other cellular kinases, such as Syk kinase. Activation of Syk kinase results in activation of downstream substrates (e.g., including PI3K) which serve as pro-inflammatory mediators.

In some aspects, the inhibitory Fc receptor is an FcγRIIB, such as an FcγRIIB1 or FcγRIIB2. FcγRII is an integral membrane glycoprotein of 40 KDa. This receptor has only two immunoglobulin-like regions in its immunoglobulin binding chain, and thus has a much lower affinity for IgG than FcγRI. FcγRII primarily binds to complexed IgG due to its lower affinity for monomeric Ig (10⁶ M⁻¹). This receptor is the most widely expressed FcγR, present on monocytes, macrophages, B cells, NK cells, neutrophils, mast cells, as well as platelets etc. There are three human FcγRII genes (FcγRII-A, FcγRII-B and FcγRII-C), all of which bind to IgG in immune complexes. However, the distinct differences within the cytoplasmic domains of FcγRII-A and FcγRII-B create two functionally distinct responses to receptor binding. The basic difference is that the A isoform initiates intracellular signaling leading to cell activation (e.g., phagocytosis and respiratory burst), whereas the B isoform initiates an inhibitory signal via an ITAM motif, e.g., suppression of B cell activation. When bound with activated FcγR, ITIM in FcγRIIB is phosphorylated and attracts the SH2 domain of inositol polyphosphate 5′-phosphatase (SHIP), which results in activation of phosphoinositol messenger (ITAM-containing FcγR-mediated tyrosine kinase) is released. This inhibits the influx of intracellular Ca++ required for further B cell activation.

Various Fc receptors and their characteristics are summarized in Table 1A.

TABLE 1A
Exemplary Receptors for Fc

| | FcγRI | FcγRIIA | FcγRIIB2 | FcγRIIB1 | FcγRIII |
|---|---|---|---|---|---|
| | (CD64) | (CD32) | (CD32) | (CD32) | (CD16) |
| Binding | IgG1 | IgG1 | IgG1 | IgG1 | IgG1 |
| Kinetics | 10⁸ M⁻¹ | 2 × 10⁶ M⁻¹ | 2 × 10⁶ M⁻¹ | 2 × 10⁶ M⁻¹ | 5 × 10⁵ M⁻¹ |
| Cell | Macrophages, | Macrophages, | Macrophages, | B cells | Eosinophils, |
| Expression | Neutrophils, | Neutrophils, | Neutrophils, | Mast cells | Macrophages, |
| | Eosinophils, | Eosinophils, | Eosinophils | | Neutrophils, |
| | Dendritic cells | Dendritic cells, | | | Mast cells |
| | | Platelets, | | | |
| | | Langerhan cells | | | |
| Effector | Uptake, | Uptake, | Uptake, | No uptake, | Induction |
| Functions | stimulation of | stimulation of | inhibition of | inhibition of | of killing |
| | respiratory burst, | granule | stimulation | stimulation | |
| | induction of | release | | | |
| | killing | | | | |

In provided embodiments, binding of the at least one binding domain, e.g. antigen-binding fragment of an antibody (e.g. containing a variable heavy (VH) chain and a variable light (VL)), to the viral surface protein neutralizes the virus. A virus can be neutralized via a binding domain (e.g., antigen binding fragment of an antibody) of any of the binding agents disclosed herein by a number of mechanisms, each resulting in overall reduction of the infectivity of a virus. In aspects of the provided embodiments, the binding agent or particle reduces or prevents viral attachment to a host cell. A binding agent bound to the surface of an extracellular virus may prevent attachment via (i) steric hindrance of viral attachment proteins, (ii) stabilization of the viral capsid, or (iii) by instigating other structural changes to surface exposed structural proteins. A binding agent bound to the surface of an extracellular virus may also neutralize virus by physical occlusion or protein interference of interactions required for endocytosis or uncoating (Rhorer et al, Vaccine, 27 (7): 1101-1110, 2009) The provided embodiments relate to methods for delivering a binding agent or particle to a subject. The binding agent or particle can be delivered as a protein or a nucleic acid agent. In some embodiments, the binding agent or particle protein (or polypeptide), such as a recombinant protein is administered to the subject. In some embodiments, a nucleic acid encoding the binding agent or particle is administered to the subject. In a particular embodiment, the agent, e.g. the protein or encoding polynucleotide, for delivering the binding agent or particle to a subject can be contained in any vehicle (e.g. viral and non-viral vectors) that can be engineered to contain the binding agent or particle or encoding nucleic acid thereof.

In some embodiments, the virus surface protein is present on the surface of a virus to neutralize or block infectivity of the virus in accord with the provided embodiments. In some embodiments, the virus comprises a DNA genome. In some embodiments, the virus comprises an RNA genome. In some embodiments, the viral genome is a single stranded nucleic acid species. In some embodiments, the viral genome is a double stranded nucleic acid species. In some embodiments, the virus is a member of any one of seven Baltimore classifications. Developed and proposed by David Baltimore, the Baltimore classifications divide viruses into major groups based on their genomic structure and replication strategy. In some embodiments, the binding agent or particle inhibits the replication of Baltimore Group I viruses. In some aspects, group I viruses comprise a double-stranded DNA genome, such as viruses belonging to Herpesviridae (e.g., Herpes simplex virus type 1), Adenoviridae (e.g., human Adenovirus), and Papovaviridae. In some embodiments, the binding agent or particle inhibits the replication of Baltimore Group II viruses. In some aspects, group II viruses comprise a single-stranded DNA genome, such as viruses belonging to Parvoviridae. In some embodiments, the binding agent or particle inhibits the replication of Baltimore Group III viruses. In some aspects, group III viruses comprise a double-stranded RNA genome, such as viruses belonging to Reoviridae and Birnaviridae. In some embodiments, the binding agent or particle inhibits the replication of Baltimore Group IV viruses. In some aspects, group IV viruses comprise a single-stranded RNA genome of the positive sense, such as viruses belonging to Coronaviridae, Flavivirdae (e.g., Dengue virus, West Nile virus, and Hepatitis C virus), Togaviridae (e.g., Chikungunya virus), and Picornaviridae. In some embodiments, the binding agent or particle inhibits the replication of Baltimore Group V viruses. In some aspects, group V viruses comprise a single-stranded RNA genome of the negative sense, such as viruses belonging to Orthomyxoviridae (e.g., Influenza viruses), Paramyxoviridae, Filoviridae, and Rhabdoviridae. In some embodiments, the binding agent or particle inhibits the replication of Baltimore Group VI viruses, such as retroviruses (e.g., lentiviruses such as HIV-1 and HIV-2). In some aspects, group VII viruses comprise a double-stranded RNA genome that requires and RNA intermediate, such as pararetroviruses (E.g., Hepatitis B). In some embodiments, the virus is within Baltimore Group IV. In some embodiments, the virus is a member of the family Coronaviridae. In some embodiments, the virus is a coronavirus.

There are currently no FDA approved treatments for SARS-CoV-1, SARS-CoV-2 or MERS. In August of 2021, the first vaccine directed towards SARS-CoV-2 was approved by the FDA for use in people over the age of 12. While other vaccines are currently administered under an Emergency Use Authorization (EUA) in the United States, there remains an immense unmet need to provide methods of preventing and treating coronavirus infection. There are no vaccines available for either of SARS CoV-1 or MERS, and no vaccines for SARS CoV-2 are currently availble for children under 12.

Also provided herein are methods and uses of the polynucleotides and vehicles provided herein, such as in therapeutic methods. Also provided are polynucleotides, compositions containing the vehicles and polynucleotides, and kits for using and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Binding Agents with Reduced Inflammatory Activity

The provided binding agents or particles contain at least one binding domain, e.g. an antigen-binding fragment, and Fc domain. Features of the provided binding agents or particles are described in the following subsections. Also provided herein are polynucleotides (nucleic acid molecules) encoding any of the provided binding agents. The polypeptide binding agents, particles, or polynucleotides encoding the binding agent, can be administered to subjects for treating or reducing a viral infection in a subject, according to the provided embodiments and methods.

In some embodiments, the provided binding agents or particles comprise an Fc domain with reduced pro-inflammatory activity as compared to an IgG1 Fc domain. In some embodiments, the provided binding agents or particles comprise a modified Fc domain, such as those with reduced affinity to an Fc activating receptor or increased affinity for an inhibitory Fc receptor, exhibit anti-inflammatory or reduced pro-inflammatory activity. In some embodiments, the provided binding agents or particles are capable or neutralizing the virus. The provided binding agents or particles can modulate immune cell activity, such as one or more of cell proliferation, differentiation, activation, or survival. In some cases, the provided binding agents may additionally modulate immune complex (IC) function. In some embodiments, activating immune complex function is reduced, decreased or attenuated. In some embodiments, inhibitory immune complex function is greater, increased or potentiated.

Human Fc receptors include two or more Ig like domains which are capable of binding the CH region of the IgG Fc domain, including wherein said Fc domain is present within an IC. Signal transduction is primarily propagated via specific phosphorylation of intracellular tyrosine activating motifs (ITAMs). Activating Fc receptors FcγRI, FcγRIIA, and FcγRIII feature ITAM sequences which can be phosphorylated by src kinase. This kinase activity results in the eventual downstream production of secondary messengers, and in turn can alter gene expression. In contrast, phosphorylation of ITIM sequences found on the intracellular domains of inhibitory Fc receptors, such as FcγRIIB1 or FcγRIIB2, results in the activation of suppressing phosphatases (Junker et al, Front. Immunol (11):1393, 2020).

The function of provided binding agents can be examined using a variety of approaches to assess the ability of the provided agents to bind to cognate binding partners as described above. For example, binding agents comprising a modified Fc domain with reduced affinity for a Fc activating receptor may be tested against FcγRI, FcγRIIA, and/or FcγRIII. In the case of binding agents comprising a modified Fc domain with increased affinity for a Fc inhibitory receptor, binding agents provided herein may be assessed for binding to the cognate binding partner FcRIIB, such as FcγRIIB1 or FcγRIIB2. A variety of assays are known for assessing binding affinity and/or determining whether a binding molecule (e.g., binding agent or particle) specifically binds to a particular binding partner. It is within the level of a skilled artisan to determine the binding affinity of a binding molecule, e.g., binding agent or particle for a binding partner, e.g., FcγR family member, such as by using any of a number of binding assays that are well known in the art. Various binding assays are known and include, but are not limited to, for example, ELISA K_{D}, KinExA, flow cytometry, and/or surface plasmon resonance devices), including those described herein. Such methods include, but are not limited to, methods involving BIAcore®, Octet®, or flow cytometry. For example, in some embodiments, a BIAcore® instrument can be used to determine the binding kinetics and constants of a complex between two proteins using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

Provided binding agents or particles also can be assessed in any of a variety of assays to assess modulation of immune cell activity. One such assay is a cell proliferation assay. Cells are cultured in the presence or absence of a test compound (e.g. binding agent), and cell proliferation is detected by, for example, measuring incorporation of titrated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. Numerous reporter genes that are easily assayed for in cell extracts are known in the art, for example, the E. coli lacZ, chloroamphenicol acetyl transferase (CAT) and serum response element (SRE) (see, e.g., Shaw et al., Cell 56:563-72, 1989). An exemplary reporter gene is a luciferase gene (de Wet et al., Mol. Cell. Biol. 7:725, 1987). Expression of the luciferase gene is detected by luminescence using methods known in the art (e.g., Baumgartner et al., J. Biol. Chem. 269:29094-101, 1994; Schenborn and Goiffin, Promega Notes 41:11, 1993). Luciferase activity assay kits are commercially available from, for example, Promega Corp., Madison, Wis.

Provided binding agents or particles can be characterized by the ability to inhibit the stimulation of cells by soluble stimulators, for example with B lymphocytes as described by Gross et al, international publication No. WO 2000/40716. Briefly, human B cells are isolated from peripheral blood mononuclear cells, such as using CD19 magnetic beads separation (e.g. Miltenyi Biotec Auburn, CA). The purified B cells can be incubated under conditions of stimulation, and further in the presence of titrated concentration of binding agent. The B cells can be labeled with a proliferation dye or can be labeled with 1 μCi ³H-thymidine to measure proliferation. The number of B cells can be determined over time. Similar assays can be performed with other immune cell types using specific soluble stimulators, and are known in the art. Reporter cell lines that express a reporter gene under the operable control of a transcription factor, such as NF-κB, NFAT-1 and AP-1 in B cells for example, can also be made. Incubation of these cells with soluble stimulatory ligands signal through the reporter genes in these constructs. The effect of provided binding agents to modulate this signaling can be assessed. Certain immune cells can also be evaluated for activation based on a secretome profile. For example, activated B cells can be measured for their ability to produce antibodies via centrifugation, column affinity chromatography, western blot etc.

Immune cell activation can additionally be assessed via cytokine production. FcγR family receptors are present on many immune cell types, exemplary cellular distributions for each of the human receptors are shown in Table 1A. Initiation of signaling through any of the activating Fc receptors FcγRI, FcγRIIA, and/or FcγRIII results in the production of pro-inflammatory signals, including cytokine secretion. Interaction of an Fc domain with an activating Fc receptor, for example, on dendritic cells (DCs) results in a pronounced increase in IL-1β, IL-6, IL-23, and TNFα. In contrast, ligation of inhibitory Fc receptor FcRIIB, such as FcγRIIB1 or FcγRIIB2, results in a decreased production of pro-inflammatory cytokines, such as those above, as well as an increase in production and secretion of anti-inflammatory cytokines, such as IL-10. Numerous methods of quantifying cytokines are known in the art, for example via specific ELISA of cell culture supernatant.

Provided binding agents or particles can also be characterized by the ability to modulate antigen uptake and presentation by immune cells, for example by Antigen Presenting Cells (APCs) such as macrophages and DCs. Ligation of the activating Fc receptors on the surface of APCs results in the ITAM dependent clathrin mediated internalization of the bound target (e.g., any of the provided binding agents, immune complexes) for proteolytic processing and eventual presentation at the cell surface. It has also been shown that ICs comprising an Fc domain with affinity for an activating receptor are preferentially shuttled away from recycling endosomes in favor of lysosomes and eventual loading into an MHC II molecule (Regnault et al., J Exp Med (189)371-380, 1999). Once present at the cell surface, antigen is capable of being recognized by T cells, thus perpetuating the immune response. In contrast, ligation of the Fc inhibitory receptors does not incite clathrin coated pit formation, as the intracellular domains of the inhibitory receptor lack necessary ITAM residues.

In the subsections below, exemplary domains and sequences of the provided binding agents are described.

A. Binding Domain

In some embodiments, the at least one binding domain is from an antibody or antigen-binding fragment of an antibody that specifically binds a viral surface protein. In some embodiments, the at least one binding domain comprises an antigen-binding fragment of an antibody that specifically binds a viral surface protein. The viral surface protein can be a protein exposed on the surface of a virus. In some embodiments, the virus surface protein is present on the surface of a virus and is a target to neutralize or block infectivity of the virus in accord with the provided embodiments. In particular embodiments, the at least one binding domain exhibits neutralizing activity against a virus having exposed on its surface the viral surface protein.

In some embodiments, the viral surface protein is exposed on the surface of a virus that is a member of any one of seven Baltimore classifications. In some embodiments, the virus comprises a DNA genome. In some embodiments, the virus comprises an RNA genome. In some embodiments, the viral genome is a single stranded nucleic acid species. In some embodiments, the viral genome is a double stranded nucleic acid species. In some embodiments, the virus is a member of any one of seven Baltimore classifications. Developed and proposed by David Baltimore, the Baltimore classifications divide viruses into major groups based on their genomic structure and replication strategy. In some embodiments, the binding agent or particle inhibits the replication of Baltimore Group I viruses. In some aspects, group I viruses comprise a double-stranded DNA genome, such as viruses belonging to Herpesviridae (e.g., Herpes simplex virus type 1), Adenoviridae (e.g., human Adenovirus), and Papovaviridae. In some embodiments, the binding agent or particle inhibits the replication of Baltimore Group II viruses. In some aspects, group II viruses comprise a single-stranded DNA genome, such as viruses belonging to Parvoviridae. In some embodiments, the binding agent or particle inhibits the replication of Baltimore Group III viruses. In some aspects, group III viruses comprise a double-stranded RNA genome, such as viruses belonging to Reoviridae and Birnaviridae. In some embodiments, the binding agent or particle inhibits the replication of Baltimore Group IV viruses. In some aspects, group IV viruses comprise a single-stranded RNA genome of the positive sense, such as viruses belonging to Coronaviridae, Flavivirdae (e.g., Dengue virus, West Nile virus, and Hepatitis C virus), Togaviridae (e.g, Chikungunya virus), and Picornaviridae. In some embodiments, the binding agent or particle inhibits the replication of Baltimore Group V viruses. In some aspects, group V viruses comprise a single-stranded RNA genome of the negative sense, such as viruses belonging to Orthomyxoviridae (e.g., Influenza viruses), Paramyxoviridae, Filoviridae, and Rhabdoviridae. In some embodiments, the binding agent or particle inhibits the replication of Baltimore Group VI viruses, such as retroviruses (e.g., lentiviruses such as HIV-1 and HIV-2). In some aspects, group VII viruses comprise a double-stranded RNA genome that requires and RNA intermediate, such as pararetroviruses (E.g., Hepatitis B). In some embodiments, the virus is within Baltimore Group IV. In some embodiments, the virus is a member of the family Coronaviridae. In some embodiments, the virus is a coronavirus.

In some aspects, the viral surface protein exposed on the surface of a virus can be any protein that includes a receptor binding domain (RBD) that mediates binding to a cognate receptor on a target host cell that can be infected by the virus. In some embodiments, the first step in any viral life cycle is contact and attachment with a target host cell, which can be mediated by structural proteins or viral membrane proteins via a surface exposed receptor binding domain (RBD). Antibodies or other binding domains which target the RBD or subvert its function are among the class of antibodies known as neutralizing, as their binding occludes virus-host receptor interaction and therefore “neutralizes” the ability to gain entry into a host cell. In some aspects, the viral surface protein is a viral membrane fusion protein. In some aspects, the viral surface protein is a structural protein, such as a capsid protein.

In some aspects, the RBD is located in a surface exposed viral membrane fusion glycoprotein. Viral fusion glycoproteins are characterized into three classes based on structure as most enveloped viruses utilize the same membrane-fusion pathway. Class I viral membrane fusion proteins project away from the membrane and feature a major structure comprising an alpha helix coiled coil. In some aspects, Class I proteins include influenza virus hemagglutinin (HA) and the fusion (F) protein of paramyxoviruses. Native Class I proteins on the surface of virions are trimeric pre and post fusion, with each monomer requiring proteolytic processing prior to fusion. The resulting C terminal fragment is anchored in the viral membrane while the fusion peptide located at or near the N terminus. Class II viral membrane fusion proteins were until recently limited to the E protein of flaviviruses and E1 of alphaviruses but now include proteins from additional families Bunyaviridae and Togaviridae. These Class II proteins do not appear as spikes, instead their orientation is majorly parallel to the membrane. The major secondary structure of each of the three globular domains is primarily B pleated sheets, with the fusion loop being internal at domain II at the interface of each of two monomers. Like Class I proteins, Class II viral membrane fusion proteins require proteolytic cleavage in order to be fusogenic. Class III viral membrane fusion proteins comprise both an alpha helix and beta pleated sheet and share features of both Classes I and II. Exemplars of this class include the trimeric fusion glycoprotein G of VSV (VSV-G) and B proteins of HSV-1 and Epstein-Barr Viruses as well as the baculovirus gp64 fusogen, however the pre and post fusion crystal structure is only known for VSV-G (Verdaguer et al., IUCrJ (1) 6:492-504, 2014).

In some aspects, the RBD is located within a surface exposed viral spike protein, or S protein. In some aspects, the binding domain specifically binds the S (spike) glycoprotein of a SARS virus.

The SARS-CoV spike (S) protein is composed of two subunits; the S1 subunit contains a receptor-binding domain that engages with the host cell receptor angiotensin-converting enzyme 2 and the S2 subunit mediates fusion between the viral and host cell membranes. The S protein plays key parts in the induction of neutralizing-antibody and T-cell responses, as well as protective immunity, during infection with SARS-CoV. Native S protein is a class I viral fusion glycoprotein that is expressed on the surface of cells in a functional homotrimer. The predicted S protein consists of a signal peptide (amino acids 1-12) located at the N terminus, an extracellular domain (amino acids 13-1,195), a transmembrane domain (amino acids 1,196-1,215) and an intracellular domain (amino acids 1,216-1,255).

A fragment that is located in the S1 subunit and spans amino acids 318-510 is considered the likely minimal receptor-binding domain (RBD) needed for interaction with angiotensin-converting enzyme 2 (ACE-2) receptor. During the interaction of RBD with the ACE-2 receptor, RBD presents a concave surface for the N terminus of the receptor peptidase, on which amino acids 445-460 anchor the entire receptor-binding loop of the RBD core (Du et al., Nat Rev Microbiol. 7(3):226-236, 2009).

Using information about the RBD and inactivated SARS-CoV along with studies of convalescent sera from recovered patients, many antibodies and binding domains thereof have been generated with specificity for the SARS CoV-2 S protein. In some aspects, these S specific antibodies were observed to block S protein interaction with the ACE2 receptor and therefore neutralize infection in humans. Selected exemplary SARS CoV-2 antibodies are shown in Table 1B. In some embodiments, the binding domain contains a variable heavy (VH) chain and a variable light (VL) of an antibody set forth in Table 1B. In some embodiments, the binding agent is modified compared to an antibody described in Table 1B such that the binding agent is an antibody containing an Fc domain as described herein. In some embodiments, the Fc domain exhibits reduced pro-inflammatory activity as compared to an IgG1 Fc domain. In some embodiments, the Fc domain, such as modified Fc domain, has decreased binding to at least one Fc activating receptor family member compared to a wild-type Fc domain, such as a wild-type IgG1 Fc domain. In some embodiments, such a modified antibody exhibits neutralizing virus activity and exhibits reduced binding to an Fc activating receptor. In some embodiments, the Fc domain, such as modified Fc domain, exhibits increased binding to an Fc inhibitory receptor compared to a wild-type Fc domain. In some embodiments, such a modified antibody exhibits neutralizing virus activity and exhibits increased binding to an Fc inhibitory receptor.

In some embodiments, the binding domain is a VHH antibody, or nanobody, that binds to a viral protein, e.g., a SARS-CoV2 S protein. In some aspects, the binding domain is the antigen binding fragment of the heavy chain only that binds to a viral protein, e.g., a SARS-CoV2 S protein. In some embodiments, the binding domain is a humanized llama VHH that binds to a viral protein, e.g., a SARS-COV2 spike protein (see, e.g., Dong et al., Emerg Microbes Infect. 9(1):1034-1036, 2020). Selected exemplary SARS CoV-2 VHH antibodies are also shown in Table 1B. In some embodiments, the binding agent contains a binding domain that is a VHH and further contains an Fc domain as described herein. In some embodiments, the Fc domain exhibits reduced pro-inflammatory activity as compared to an IgG1 Fc domain. In some embodiments, the Fc domain, such as modified Fc domain, has decreased binding to at least one Fc activating receptor family member compared to a wild-type Fc domain, such as a wild-type IgG1 Fc domain. In some embodiments, such a binding agent exhibits neutralizing virus activity and exhibits reduced binding to an Fc activating receptor. In some embodiments, the Fc domain, such as modified Fc domain, exhibits increased binding to an Fc inhibitory receptor compared to a wild-type Fe domain. In some embodiments, such a binding agent exhibits neutralizing virus activity and exhibits increased binding to an Fc inhibitory receptor.

TABLE 1B
Exemplary Anti-SARS CoV S Protein Antibodies

| | | Sequences (SEQ ID NO) | |
|---|---|---|---|
| | | Variable | Variable |
| Designation | Developer | Light (VL) | Heavy (VH) |
| Monoclonal Antibodies | | | |
| STI-1499 | Sorrento/Mount Sinai | | |
| STI-4398 | Sorrento/Mount Sinai | | |
| STI-2020 | Sorrento/Mount Sinai | | |
| REGN10933 | Regeneron/NIAID | 49 | 50 |
| REGN10987 | Regeneron/NIAID | 51 | 52 |
| JS016/CB6 | Junshi Biosciences/Institute of Microbiology, | 53 | 54 |
| | Chinese Academy | | |
| LY-CoV555/ | AbCellera/Eli Lilly/NIH | | |
| LY-3819253/ | | | |
| Bamlanivimab | | | |
| TB181-8 | Twist Biopharma | | |
| TB181-28 | Twist Biopharma | | |
| TB181-36 | Twist Biopharma | | |
| BGB-DXP593 | BeiGene/Singlomics/Peking University | | |
| TY027 | Tychan | | |
| CT-P59 | Celltrion | | |
| BRII-196 | Brii Bio/TSB Therapeutics/Tsinghua University | | |
| BRII-198 | Brii Bio/TSB Therapeutics/Tsinghua University | | |
| SCTA01 | Sinocelltech/Chinese Academy of Sciences | | |
| MW33 | Mabwell (Shanghai) Bioscience | | |
| AZD8895 | AstraZeneca/Vanderbilt | | |
| AZD1061 | AstraZeneca/Vanderbilt | | |
| HLX70 | Hengenix Biotech | | |
| 15G11 | Abveris | 55 | 56 |
| 18F4 | Abveris | 57 | 58 |
| 1E5 | Abveris | 59 | 60 |
| 1G3 | Abveris | 61 | 62 |
| 21C3 | Abveris | 63 | 64 |
| 22D9 | Abveris | 65 | 66 |
| 23D11 | Abveris | 67 | 68 |
| 26E2 | Abveris | 69 | 70 |
| 29F7 | Abveris | 71 | 72 |
| 3B3 | Abveris | 73 | 74 |
| 3F2 | Abveris | 75 | 76 |
| D59047-11955 | Abveris | 77 | 78 |
| D70678-12637-S1 | Abveris | 79 | 80 |
| D70678-12799-S1 | Abveris | 81 | 82 |
| D70678-13531-S1 | Abveris | 83 | 84 |
| D70678-13576-S1 | Abveris | 85 | 86 |
| D70678-14004-S2 | Abveris | 87 | 88 |
| D70678-14027-S2 | Abveris | 89 | 90 |
| D70678-2155-S1 | Abveris | 91 | 92 |
| D70678-2743-S1 | Abveris | 93 | 94 |
| D70678-5521-S2 | Abveris | 95 | 96 |
| Single Domain Antibodies (VHH) | | | |
| TB201-1 | Twist Biopharma | | |
| TB202-3 | Twist Biopharma | | |
| TB202-63 | Twist Biopharma | | |

In some embodiments, the binding domain in accord with the provided embodiments is an antigen binding domain of an antibody that specifically binds a viral surface protein, such as any as described. The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH). Typically an antigen binding domain

In some embodiments, the binding domain is an antigen binding domain of an antibody that specifically binds SARS CoV-2. In some embodiments, the binding domain is an antigen binding domain of an antibody that specifically binds a variant of SARS CoV-2, such as a Variant of Interest (Vol), Variant of Concern (VoC), and/or Variant of High Consequence (VoHC). In some embodiments, the SARS CoV-2 variant is chosen from the group comprising: Alpha (i.e., B.1.1.7), Beta (i.e., B.1.351, B.1.351.2, B.1.351.3), Delta (i.e., B.1.617.2, AY.1, AY.2, AY.3), and Gamma (i.e., P.1, P.1.1, P.1.2).

In some embodiments, the binding domain is an antigen binding domain of an antibody selected from among STI-1499, STI-4398, REGN10933, REGN10987, REGN-COV2, JS016, LY-CoV555, LY-3819253, TB181-8, TB181-28, TB181-36, TB201-1, TB202-3, TB202-63, BGB-DXP593, TY027, CT-P59, BRII-196, BRII-198, SCTA01, MW33, AZD8895, AZD1061, HLX70, 15G11, 18F4, 1E5, 1G3, 21C3, 22D9, 23D11, 26E2, 29F7, 3B3, 3F2, D59047-11955, D70678-12637-S1, D70678-12799-S1, D70678-13531-S1, D70678-14004-S2, D70678-14027-S2, D70678-2155-S1, D70678-2743-S1, and D70678-5521-S2. In some embodiments, the binding domain contains the heavy and light chain complementarity determining regions (CDRs) of an antibody selected from STI-1499, STI-4398, REGN10933, REGN10987, REGN-COV2, JS016, LY-CoV555, LY-3819253, TB181-8, TB181-28, TB181-36, TB201-1, TB202-3, TB202-63, BGB-DXP593, TY027, CT-P59, BRII-196, BRII-198, SCTA01, MW33, AZD8895, AZD1061, HLX70, 15G11, 18F4, 1E5, 1G3, 21C3, 22D9, 23D11, 26E2, 29F7, 3B3, 3F2, D59047-11955, D70678-12637-S1, D70678-12799-S1, D70678-13531-S1, D70678-14004-S2, D70678-14027-S2, D70678-2155-S1, D70678-2743-S1, and D70678-5521-S2. In some embodiments, the binding domain contains the variable heavy (VH) and the variable light (V_{L}) regions of an antibody selected from STI-1499, STI-4398, STI-2020, REGN10933, REGN10987, REGN-COV2, JS016, LY-CoV555, LY-3819253, TB181-8, TB181-28, TB181-36, TB201-1, TB202-3, TB202-63, BGB-DXP593, TY027, CT-P59, BRII-196, BRII-198, SCTA01, MW33, AZD8895, AZD1061, HLX70, 15G11, 18F4, 1E5, 1G3, 21C3, 22D9, 23D11, 26E2, 29F7, 3B3, 3F2, D59047-11955, D70678-12637-S1, D70678-12799-S1, D70678-13531-S1, D70678-14004-S2, D70678-14027-S2, D70678-2155-S1, D70678-2743-S1, and D70678-5521-S2. In some embodiments, the binding domain is an antigen-binding fragment of an antibody selected from among STI-1499, STI-4398, STI-2020, REGN10933, REGN10987, REGN-COV2, JS016, LY-CoV555, LY-3819253, TB181-8, TB181-28, TB181-36, TB201-1, TB202-3, TB202-63, BGB-DXP593, TY027, CT-P59, BRII-196, BRII-198, SCTA01, MW33, AZD8895, AZD1061, HLX70, 15G11, 18F4, 1E5, 1G3, 21C3, 22D9, 23D11, 26E2, 29F7, 3B3, 3F2, D59047-11955, D70678-12637-S1, D70678-12799-S1, D70678-13531-S1, D70678-14004-S2, D70678-14027-S2, D70678-2155-S1, D70678-2743-S1, and D70678-5521-S2 in which the antigen-binding fragment binds to SARS CoV2.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (VH) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 2, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 2
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . | H26--H35B | H30--H35B |
| (Kabat | | 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, such as the CDR as defined by the Kabat, Chothia, AbM or Contact method, or other known schemes. In some embodiments, the CDRs are defined by Kabat numbering.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or VL domain may be sufficient to confer antigen-binding specificity.

Among the provided binding domains are antigen binding domains that are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (VH) regions, single-chain antibody molecules such as scFvs and single-domain antibodies comprising only the VH region; and multispecific antibodies formed from antibody fragments. In some embodiments, the antibody is or comprises an antibody fragment comprising a variable heavy chain (VH) and a variable light chain (V_{L}) region. In particular embodiments, the antibodies are single-chain antibody fragments comprising a heavy chain variable (VH) region and/or a light chain variable (V_{L}) region. For example, a single chain variable fragment (scFv) contains a heavy chain variable (V_{H}) region and a light chain variable (V_{L}) region, typically joined by a peptide linker.

Single-domain antibodies (sdAbs) are antibody fragments comprising all or a portion of the heavy chain variable region or all or a portion of the light chain variable region of an antibody. In certain embodiments, a single-domain antibody is a human single-domain antibody. In certain embodiments, a single-domain antibody is a camelid antibody, e.g., a camel, llama, or alpaca antibody. In certain embodiments, a single-domain antibody is a humanized camelid antibody, e.g., a humanized camel, humanized llama, or humanized alpaca antibody.

Antibody fragments can be made by various techniques, including but not limited to proteolytic digestion of an intact antibody as well as production by recombinant host cells. In some embodiments, the antibodies are recombinantly-produced fragments. Such fragments can include arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

Among the provided antibodies are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In particular embodiments, the binding domain is an antigen-binding fragment of an antibody that contains a variable heavy chain (VH) and a variable light chain (VL) of an antibody. In some embodiments, the binding agent is a two-chain molecule in which the VH chain and VL chain are linked by a disulfide bond. In some embodiments, the binding agent is a single chain molecule in which the VH chain and VL chain are joined by a linker, such as a peptide linker. In some embodiments, the binding domain is a Fab fragment of an antibody. In some aspects, a Fab is composed of one constant and one variable domain of each of the heavy and light chains of an antibody. In some embodiments, the antigen-binding fragment is selected from among a Fab fragment, F(ab′)₂ fragment, Fab′ fragment, Fv fragment, or a scFv. F(ab′)2 fragment antibodies lack most of the Fc region of whole IgG antibodies while leaving a small portion of the hinge region not present in traditional Fab fragments. F(ab′)2 fragments have two antigen-binding F(ab) portions linked together by disulfide bonds, with a molecular weight of about 110 kDa. Single-chain variable fragments (scFvs) are recombinant molecules in which the variable regions of light and heavy immunoglobulin chains encoding antigen-binding domains are engineered into a single polypeptide. In some aspects, the VH and VL sequences are joined by a flexible linker sequence.

In some embodiments, the binding domain is a single domain antibody, such as a VHH antibody, including those derived from camelids or llamas.

In some aspects, the binding domain is an antibody mimetic, such as selected from Designed Ankyrin Repeat Protein (DARPin), adnectins, or an antigen-binding fibronectin type III (Fn3) scaffold.

B. Fe Domains

Provided binding agents herein include the at least one binding domain joined (e.g. linked directly or linked indirectly via a linker) to an Fc domain. In some embodiments, the Fc is modified. In provided embodiments, the Fc domain of the binding agent does not exhibit pro-inflammatory activity or exhibits reduced pro-inflammatory activity compared to a wild-type IgG1 Fc.

An Fc (fragment crystallizable) region or domain of an immunoglobulin molecule (also termed an Fc polypeptide) corresponds largely to the constant region of the immunoglobulin heavy chain, and is responsible for various functions, including the antibody's effector function(s). The Fc domain contains part or all of a hinge domain of an immunoglobulin molecule plus a CH2 and a CH3 domain. The Fc domain can form a multimer of polypeptide chains joined by one or more disulfide bonds. In some embodiments, the binding agent is a two chain antigen-binding fragment containing the light chain and the heavy chain, e.g. is a Fab, and the Fc domain is joined to the heavy chain of the binding domain, thereby forming a molecule containing two heavy chains and two light chains. In some embodiments, the binding agent is a single chain antigen-binding fragment containing the light chain and the heavy chain, e.g. is an scFv, and the Fc domain is joined to the N-terminus or C-terminus of the single chain antigen-binding fragment, thereby forming a dimer molecule containing two single chain antigen-binding fragments.

In some embodiments, the Fc domain of the provided binding agents is not a wild-type IgG1 Fc. In some embodiments, the Fc domain of the provided binding agent is not an Fc domain that contains the amino acid sequence set forth in SEQ ID NO:1.

In some embodiments, the binding agent contains an Fc domain that exhibits reduced binding to an Fc activating receptor compared to a wild-type IgG1 Fc, such as compared to an Fc domain set forth in SEQ ID NO:1. In some embodiments, the Fc domain of the provided binding agents is a an IgG2 Fc domain (e.g. set forth in SEQ ID NO:2). In some embodiments, the Fc domain of the provided binding agents is an IgG4 Fc domain (e.g. set forth in SEQ ID NO:3). In some embodiments, the binding agent contains a Fc, e.g. modified Fc domain containing one or more amino acid substitutions compared to a wild-type Fc domain, such as compared to an IgG1, lgG2, or lgG4 Fc domain, in which the modified Fc domain exhibits reduced binding to an activating Fc receptor compared to the wild-type Fc. In some embodiments, the binding agent contains a Fc, e.g. modified Fc domain that has an decreased binding affinity for a Fc activating receptor, relative to a wild-type Fc domain. In some embodiments, the Fc, e.g. modified Fc of the binding agent has a decreased binding affinity for a Fc activating receptor, relative to wild-type IgG1 Fc. In some embodiments, the decreased binding affinity is for Fc gamma receptor I (FcγRI), Fc gamma receptor IIA (FcγRIIA) and/or Fc gamma receptor III (FcγRIII) Fc domain. In some embodiments, the decrease in binding affinity relative to the wild-type Fc domain control, such as wild-type IgG1 Fc, is decreased by at least about 5%, such as at least about 10%, 15%, 20%, 25%, 35%, or 50%. In some embodiments, the decrease in binding affinity relative to the wild-type Fe domain, such as wild-type IgG1, is decreased more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold.

In some embodiments, the binding agent contains an Fc, e.g. modified Fc that exhibits increased binding to an Fc inhibitory receptor compared to a wild-type Fc. In some embodiments, the binding agent contains a Fc, e.g. modified Fc domain containing one or more amino acid substitutions compared to a wild-type Fc domain, such as compared to an IgG1, lgG2, or lgG4 Fc domain, in which the modified Fc domain exhibits increased binding to an inhibitory Fc receptor compared to the wild-type Fc. In some embodiments, the wild-type Fc domain is a wild-type IgG1 Fc. In some embodiments, the binding agent comprising a Fc, e.g. modified Fc domain has an increased binding affinity for a Fc inhibitory receptor, relative to the wild-type Fc domain. In some embodiments, the Fc, e.g. modified Fc domain has an increased binding affinity for FcγRIIB, e.g. FcγRIIB1 or FcγRIIB2, relative to the wild-type Fc domain. In some embodiments, a binding agent comprising a Fc, e.g. modified Fc domain with increased or greater binding affinity to a Fc inhibitory receptor will have an increase in binding affinity relative to the wild-type Fc domain control of at least about 5%, such as at least about 10%, 15%, 20%, 25%, 35%, or 50%. In some embodiments, the increase in binding affinity relative to the wild-type Fc domain is increased more than 1.2-fold, 1.5-fold, 2-fold, 3-fold, 4-fold, 5-fold, 6-fold, 7-fold, 8-fold, 9-fold, 10-fold, 20-fold, 30-fold 40-fold or 50-fold.

In some embodiments, the wild-type Fc domain is human Fc. In some embodiments, the wild-type Fc domain is a mammalian or human IgG1, lgG2, or lgG4 Fc regions.

In some embodiments, the wild-type Fc domain is derived from IgG1, such as human IgG1. In some embodiments, the wild-type Fc domain is an IgG1 Fc set forth in SEQ ID NO: 1. In some embodiments, the wild-type Fc domain comprises the amino acid sequence set forth in SEQ ID NO: 1 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 1.

In some embodiments, the wild-type Fc domain is derived from IgG2, such as human IgG2. In some embodiments, the wild-type Fc domain is an IgG2 Fc t set forth in SEQ ID NO: 2. In some embodiments, the wild-type Fc domain comprises the amino acid sequence set forth in SEQ ID NO: 2 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2.

In some embodiments, the wild-type Fe domain is derived from IgG4, such as human IgG4. In some embodiments, the wild-type Fc domain is an IgG4 Fc t set forth in SEQ ID NO: 3. In some embodiments, the wild-type Fc domain comprises the amino acid sequence set forth in SEQ ID NO: 3 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 3.

In some of any of the provided embodiments, the Fc, e.g. modified Fc sequence has the sequence of the wild-type Fc sequence, such as any described above (e.g., the Fc region of wild-type IgG1, IgG2, or IgG4), but additionally contains one more amino acid substitutions. Any amino acid substitution that reduces binding to an activating FcR or increases binding to an inhibitory Fc receptor are contemplated. Reference to amino acid substitutions in an Fc region is by EU numbering system unless described with reference to a specific SEQ ID NO. EU numbering is known and is according to the most recently updated IMGT Scientific Chart (IMGT®, the international ImMunoGeneTics information system®, www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

In some embodiments, the Fc, e.g. modified Fc domain has up to 1, 2, 3, 4, 5, 6, 7, 8, 9, 10, 11, 12, 13, 14, 15, 16, 17, 18, 19, or 20 amino acid substitution(s). In some embodiments, the Fc, e.g. modified Fc containing the one or more amino acid substitutions as described has at least about 85%, 86%, 86%, 88%, 89%, 90%, 91%, 92%, 93%, 94%, 95%, 96%, 97%, 98%, or 99% sequence identity with the wild-type Fc domain, such as with the amino acid sequence of SEQ ID NO: 1, 2, or 3. In some embodiments, the Fc, e.g. modified Fc domain containing the one or more amino acid substitutions as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 1. In some embodiments, the Fc, e.g. modified Fc containing the one or more amino acid substitutions as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 2. In some embodiments, the Fc, e.g. modified Fc domain containing the one or more amino acid substitutions as described has at least about 85%, 86%, 86%, 88%, 89%, 90%, 91%, 92%, 93%, 94%, 95%, 96%, 97%, 98%, or 99% sequence identity with the amino acid sequence of SEQ ID NO: 3.

In some embodiments, a binding agent comprising a Fc, e.g. modified Fc domain has a binding affinity for one or any of the FcγR family members that differs from that of the wild-type Fc sequence as determined by, for example, solid-phase ELISA immunoassays, flow cytometry or Biacore assays.

Exemplary modifications, such as amino acid substitutions, in an Fc domain are described in the following subsections.

1. Modifications to Activating Fc Domains

In some embodiments, the Fc region contains one more modifications, such as one or more amino acid substitutions, to alter one or more of its normal functions. In general, the Fc region is responsible for effector functions via binding of Fc to an activating Fc receptor, such as Fc-dependent cytokine release and antibody-dependent cell cytotoxicity (ADCC), in addition to the antigen-binding capacity, which is the main function of immunoglobulins. For example, signaling through FcγRs activates immune cells to secrete various pro-inflammatory cytokines such as IFN-gamma, monocyte chemoattractant protein-1, IL-6, tumor necrosis factor (TNF), GM-CSF, or IL-8. Additionally, the FcRn sequence present in the Fc region plays the role of regulating the IgG level in serum by increasing the in vivo half-life by conjugation to an in vivo FcRn receptor. In some embodiments, such functions can be reduced or altered in an Fc for use with the provided binding agents.

In some embodiments, one or more amino acid substitutions may be introduced into the Fc region, thereby generating a modified Fc domain. In some embodiments, the modified Fc domain has decreased effector function. There are many examples of changes or mutations to Fc sequences that can alter effector function. For example, WO 00/42072, WO2006019447, WO2012125850, WO2015/107026, US2016/0017041 and Shields et al. J Biol. Chem. 9(2): 6591-6604 (2001) describe exemplary Fc variants with improved or diminished binding to FcRs. The contents of those publications are specifically incorporated herein by reference.

In some embodiments, the provided binding agent comprise an Fc region that exhibits reduced effector functions, which makes it a desirable candidate for applications in which certain effector functions (such as ADCC or Fc-dependent cytokine release) are unnecessary or deleterious. In vitro and/or in vivo cytotoxicity assays can be conducted to confirm the reduction/depletion of ADCC activities or Fc-dependent cytokine release. For example, Fc receptor (FcR) binding assays can be conducted to ensure that the binding agent lacks FcγR binding, but retains FcRn binding ability. The primary cells for mediating ADCC, NK cells, express FcγRIII only, whereas monocytes express FcγRI, FcγRII and FcγRIII. FcR expression on hematopoietic cells is summarized in Table 2 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). FcRn binding and in vivo clearance/half-life determinations can also be performed using methods known in the art (see, e.g., Petkova, S. B. et al., Int'l. Immunol. 18(12):1759-1769 (2006)).

Binding agent with reduced effector function include those with substitution of one or more of Fc region residues 238, 265, 269, 270, 297, 327 and 329 by EU numbering (U.S. Pat. No. 6,737,056). Such Fc mutants include Fc mutants with substitutions at two or more of amino acid positions 265, 269, 270, 297 and 327 by EU numbering, including the so-called “DANA” Fc mutant with substitution of residues 265 and 297 to alanine (U.S. Pat. No. 7,332,581).

In some aspects, a wild-type Fc is modified by one or more amino acid substitutions to reduce effector activity or to render the Fc inert for Fc effector function. Exemplary effectorless or inert mutations include those described herein. In some embodiments, the Fc region of binding agent has an Fc region in which any one or more of amino acids at positions 233, 234, 235, 236, 237, 238, 239, 265, 268, 270, 288, 297, 298, 309, 318, 328, 330, 325, 328, 329, 330 and 331 (indicated by EU numbering) are substituted with different amino acids compared to the native Fc region.

Such alterations of Fc region are not limited to the above-described alterations, and include, for example, alterations such as deglycosylated chains (N297A and N297Q), IgG1-N297G, IgG1-L234A/L235A, IgG1-L234A/L235E/G237A, IgG1-A325A/A330S/P331S, IgG1-C226S/C229S, IgG1-C226S/C229S/E233P/L234V/L235A, IgG1-E233P/L234V/L235A /G236del/S267K, IgG1-L234F/L235E/P331S, IgG1-S267E/L328F, IgG2-V234A/G237A, IgG2-H268Q/V309L/A330S/A331S, IgG4-L235A/G237A/E318A, and IgG4-L236E described in Current Opinion in Biotechnology (2009) 20 (6), 685-691; alterations such as G236R/L328R, L235G/G236R, N325A/L328R, and N325LL328R described in WO 2008/092117; amino acid insertions at positions 233, 234, 235, and 237 (indicated by EU numbering); and alterations at the sites described in WO 2000/042072. Certain Fc variants with improved or diminished binding to FcRs are described. (See, e.g., U.S. Pat. No. 6,737,056; WO 2004/056312, WO2006019447 and Shields et al., J. Biol. Chem. 9(2): 6591-6604 (2001).)

Antibodies with increased half-lives and improved binding to neonatal Fc receptor (FcRn) are known in the art. FcRn are described in US2005/0014934A1 (Hinton et al.) or WO2015107026. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434 by EU numbering, e.g., substitution of Fc region residue 434 (U.S. Pat. No. 7,371,826).

In some embodiments, the Fc is an IgG4 containing the Ser228Pro mutation, which has been shown to reduce affinity for the FcγR1 affinity (see e.g. Saunders et al., Front Immunol. (10); 1296, 2019). In some embodiments, the Fc is an IgG4 containing the Leu235Glu mutation. In some embodiments, the Fc is an IgG4 containing the Ser228Pro mutation and the Leu235Glu mutation (Ser288Pro/Leu235Glu). In some embodiments, the modified Fc domain comprises a Ser228Pro substitution based on EU numbering, Phe234Ala substitution based on EU numbering, and Leu235Ala substitution based on EU numbering (Ser228Pro/Phe234Ala/Leu235Ala).

In some embodiments, the modified Fc domain comprises a modified IgG1 domain comprising an amino acid substitution selected from, Glu233Pro, Leu234Ala, Leu234Glu, Leu235Ala, Leu235Phe, Gly236Arg, Gly237Ala, Pro238Ser, Asp265Ala, His268Ala, His268Gln, Ser288Pro, Asn297Ala, Asn297Gly, Asn297Gln, Val309Leu, Gly318Ala, Leu328Arg, Pro329Gly, Ala330Ser, and Pro331Ser, each based on EU numbering, or combinations of any of the foregoing.

In some embodiments, the modified Fe domain comprises a Leu234Ala substitution based on EU numbering and Leu235Ala substitution based on EU numbering (Leu234A/Leu235A). In some embodiments, the modified Fe domain comprises a Leu234Ala substitution based on EU numbering, Leu235Ala substitution based on EU numbering, and Pro329Gly substitution based on EU numbering (Leu234Ala/Leu235Ala/Pro329Gly). In some embodiments, the modified Fe domain comprises a Pro331Ser substitution based on EU numbering, Leu234Glu substitution based on EU numbering, and Leu235Phe substitution based on EU numbering (Pro331Ser/Leu234Glu/Leu235Phe). In some embodiments, the modified Fe domain comprises a Asp265Ala substitution based on EU numbering. In some embodiments, the modified Fe domain comprises a Gly237Ala substitution based on EU numbering. In some embodiments, the modified Fe domain comprises a Gly318Ala substitution based on EU numbering. In some embodiments, the modified Fe domain comprises a Glu233Pro substitution based on EU numbering. In some embodiments, the modified Fe domain comprises a Gly236Arg substitution based on EU numbering, Leu328Arg substitution based on EU numbering, and Pro329Gly substitution based on EU numbering (Gly236Arg/Leu328Arg/Pro329Gly). In some embodiments, the modified Fe domain comprises a Leu234Ala substitution based on EU numbering, Leu235Ala substitution based on EU numbering, Gly237Ala substitution based on EU numbering, Pro238Ser substitution based on EU numbering, His268Ala substitution based on EU numbering, Ala330Ser substitution based on EU numbering, and Pro331Ser substitution based on EU numbering (Leu234Ala/Leu235Ala /Gly237Ala/Pro238Ser/His268Ala/Ala330Ser/Pro331Ser). In some embodiments, the modified Fe domain comprises a Asn297Ala substitution based on EU numbering, Asn297Gly substitution based on EU numbering, or Asn297Gln substitution based on EU numbering.

2 Modifications to Inhibiting Fc Domains

In some embodiments, the Fc region contains one more modifications, such as one or more amino acid substitutions, to alter one or more of its normal functions. In some aspects, the Fc region is responsible for effector functions via binding of Fe to an activating Fc receptor, such as Fc-dependent cytokine release and antibody-dependent cell cytotoxicity (ADCC), in addition to the antigen-binding capacity, which is the main function of immunoglobulins as is described above in Section 1.B.1. Alternatively, the Fc region can also participate in effector functions via ligation of an inhibitory Fc receptor, such as the inhibitory Fc receptor (FcγRIIB).

In some embodiments, there is provided a binding agent comprising a variant Fc region comprising one or more amino acid substitutions which improve binding to the inhibitory Fc receptor (FcγRIIB).

In some aspects, a wild-type Fc is modified by one or more amino acid substitutions to increase affinity for Fc inhibitory receptors and reduce pro-inflammatory signaling.

In some aspects, a wild-type Fc is modified by one or more amino acid substitutions to increase affinity for Fc inhibitory receptors and/or display increased glycan sialylation. In some embodiments, the modified Fc domain comprises a modified IgG1 domain comprising an amino acid substitution, Phe241Ala. In some aspects, a modified IgG1 domain comprising an amino acid substitution Phe241Ala increases glycan sialyation such that increased conformational flexibility of the Fc C_{H}2 domain is observed. In some aspects, amino acid substitution Phe241Ala increases affinity for Fc inhibitory receptors and/or stimulates expression of inhibitory Fc receptors (Ahmed et al., Jr Mol Biol (426) 18: 3166-3179, 2014). In some aspects, amino acid substitution Phe241Ala increases affinity for Fc inhibitory receptors and reduces pro-inflammatory signaling.

Exemplary effectorless or inert mutations include those described herein. In some embodiments, the Fc region of binding agent has an Fc region in which any one or more of amino acids at positions 233, 236, 237, 239, 267, 268, 270, 271, 330 and 332 (indicated by EU numbering) are substituted with different amino acids compared to the native Fc region.

Such alterations of Fc region are not limited to the above-described alterations. Certain Fc variants with improved binding to inhibitory FcRs are described. (See, e.g., Mimoto et al., Protein Engineering, (26):10 589-598, 2013; Saunders et al., Front Immunol. (10); 1296, 2019).

In some embodiments, the modified Fc domain comprises a modified IgG1 domain comprising an amino acid substitution selected from, Glu233Asp, Gly237Asp, Ser267Glu, His268Phe, His268Asp, Pro271Glu, Pro271Gly, Ala330Arg, Leu328Phe, Ser324Thr, Pro238Asp, Leu328Glu, Ser239Asp, Ile332Glu, Gly236Ala each based on EU numbering, or combinations of any of the foregoing.

In some embodiments, the modified Fc domain comprises a Ser267Glu substitution based on EU numbering and His268Phe substitution based on EU numbering, and Ser324Thr substitution based on EU numbering (Ser267Glu/His268Phe/Ser324Thr). In some embodiments, the modified Fc domain comprises a Ser267Glu substitution based on EU numbering and Leu328Phe substitution based on EU numbering (Ser267Glu/Leu328Phe). In some embodiments, the modified Fe domain comprises a Pro238Asp substitution based on EU numbering. In some embodiments, the modified Fe domain comprises a Leu328Glu substitution based on EU numbering. In some embodiments, the modified Fe domain comprises a Ser239Asp substitution based on EU numbering and Ile332Glu substitution based on EU numbering (Ser239Asp/Ile332Glu). In some embodiments, the modified Fe domain comprises a Ser239Asp substitution based on EU numbering and Ile332Glu substitution based on EU numbering, and Gly236Ala substitution based on EU numbering (Ser239Asp/Ile332Glu/Gly236Ala). In some embodiments, the modified Fe domain comprises a Ser267Glu substitution based on EU numbering. In some embodiments, the modified Fe domain comprises a Glu233Asp substitution based on EU numbering. In some embodiments, the modified Fe domain comprises a Gly237Asp substitution based on EU numbering. In some embodiments, the modified Fe domain comprises a His268Asp substitution based on EU numbering. In some embodiments, the modified Fe domain comprises a Pro271Glu substitution based on EU numbering. In some embodiments, the modified Fe domain comprises a Ala330Arg substitution based on EU numbering. In some embodiments, the modified Fe domain comprises a Glu233Asp substitution based on EU numbering and a Ala330Arg substitution based on EU numbering (Glu233Asp/Ala330Arg). In some embodiments, the modified Fe domain comprises a Glu233Asp substitution based on EU numbering, a Pro271Gly substitution based on EU numbering, and a Ala330Arg substitution based on EU numbering (Glu233Asp/Pro271Gly/Ala330Arg). In some embodiments, the modified Fe domain comprises a Gly237Asp substitution based on EU numbering, a His268Asp substitution based on EU numbering, and a Pro271Gly substitution based on EU numbering (Gly237Asp/His268Asp/Pro271Gly). In some embodiments, the modified Fe domain comprises a Gly237Asp substitution based on EU numbering, a Pro271Gly substitution based on EU numbering, and a Ala330Arg substitution based on EU numbering (Gly237Asp/Pro271Gly/Ala330Arg). In some embodiments, the modified Fe domain comprises a Glu233Asp substitution based on EU numbering, a His268Asp substitution based on EU numbering, a Pro271Gly substitution based on EU numbering, and a Ala330Arg substitution based on EU numbering (Glu233Asp/His268Asp/Pro271Gly/Ala330Arg). In some embodiments, the modified Fe domain comprises a Gly237Asp substitution based on EU numbering, a His268Asp substitution based on EU numbering, a Pro271Gly substitution based on EU numbering, and a Ala330Arg substitution based on EU numbering (Gly237Asp/His268Asp/Pro271Gly/Ala330Arg). In some embodiments, the modified Fe domain comprises a Glu233Asp substitution based on EU numbering, a Gly237Asp substitution based on EU numbering, a His268Asp substitution based on EU numbering, a Pro271Glu substitution based on EU numbering, and a Ala330Arg substitution based on EU numbering (E233D/Gly237Asp/His268Asp/Pro271Glu/Ala330Arg).

C. Linker

The linkage between the one or more binding domains disclosed in Section I.A and the one or more Fc (e.g., modified Fc) disclosed in Section I.B may be linked directly or indirectly, i.e. the binding domain peptides and the Fc domain directly adjoin or they may be linked by an additional component of the complex, e.g. a spacer or a linker.

A direct linkage may be realized by an amide bridge, such as if the components to be linked have reactive amino or carboxy groups. More specifically, if the components to be linked are peptides, polypeptides or proteins, linkage may be via a peptide bond. Such a peptide bond may be formed using a chemical synthesis involving both components (an N-terminal end of one component and the C-terminal end of the other component) to be linked, or may be formed directly via a protein synthesis of the entire peptide sequence of both components, wherein both (protein or peptide) components are preferably synthesized in one step. Such protein synthesis methods include e.g., without being limited thereto, liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc. Alternatively, ester or ether linkages are possible.

Moreover, in particular if the components to be linked are peptides, polypeptides or proteins, a linkage may occur via the side chains, e.g. by a disulfide bridge. The linkage via a side chain is based on a side chain amino, thiol or hydroxyl group, e.g. via an amide or ester or ether linkage. A linkage of a peptidic main chain with a peptidic side chain of another component may also be via an isopeptide bond. An isopeptide bond is an amide bond that is not present on the main chain of a protein. The bond forms between the carboxyl terminus of one peptide or protein and the amino group of a lysine residue on another (target) peptide or protein.

The binding particles disclosed herein may comprise a spacer or linker, which are non-immunologic moieties. particle. Examples of further functionalities, in particular regarding linkers in fusion proteins for example, can be found in Chen X. et al., 2013: Fusion Protein Linkers: Property, Design and Functionality. Adv Drug Deliv Rev. 65(10): 1357-1369, wherein for example also in vivo cleavable linkers are disclosed. Moreover, Chen X. et al., 2013: Fusion Protein Linkers: Property, Design and Functionality. Adv Drug Deliv Rev. 65(10): 1357-1369 also discloses various linkers, e.g. flexible linkers and rigid linkers, and linker designing tools and databases, which can be useful according to the provided embodiments or to design a linker to be used according to the provided embodiments.

In some embodiments, the Fc, e.g. modified Fc, is linked indirectly to the biding domain via a peptide linker. In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS, GGGGGS or combinations thereof. In some embodiments, the polypeptide linker has the sequence (GmS)n (SEQ ID NO:4), wherein each of m and n is 1 to 4.

In particular embodiments, the linker is a human antibody hinge domain, e.g., a human IgG1 hinge domain, a human IgG2 hinge domain, a human IgG3 hinge domain, or a human IgG4 hinge domain.

D. Particle Comprising a Binding Agent

In some embodiments, any of the provided binding agents as described in Section I can be formatted or provided as a particle, with a viral protein capable of being bound by the at least one binding domain of the particle. In some embodiments, the viral protein is bound by the at least one binding domain of the particle.

In some aspects, the provided particles can interact, wherein binding domains from independent particles bind antigen, such as multiple antigens, to form multiprotein complexes, such as immune complexes (ICs). Also called antigen-antibody complexes, ICs are formed from the binding of multiple antigens to antibodies or antigen binding fragment thereof, wherein the IC can function as a singular unit in subsequent immune responses. ICs in vivo may participate in complement deposition, opsonization, phagocytosis and/or peptide processing (Monsalvo et al., Nat Med (17)2:195-199, 2011). In some aspects, ICs have immunoregulatory function. In some aspects, ICs can be associated with tissue pathologies, such as pulmonary IC deposition as a result of respiratory viral infection, including as a result of coronavirus infection (Fu et al., Viro Sin (35):266-271, 2020).

In some aspects, IC formation is influenced by several factors. Ratio of antigen to binding domain, such as the ratio between at least one binding domain of any of the provided particles and a viral protein, as well as affinity of the Fc domain for Fc receptor can determine the size and shape of IC. In some aspects, stability of an IC is dependent on bonding between Fc domains, e.g., modified Fc domain of the provided particles, and Fc family receptors. In some aspects, IC effector function is potentiated with IC size, i.e., an increasing number of participatory particles provided herein or of participatory binding domains thereof. The stoichiometry of IC is such that antigen excess, such as an excess of viral protein, or binding agent excess, such as an excess of any of the particles provided herein, favors the formation of smaller ICs wherein fewer Fc receptors can be engaged. In some aspects, FcR cross linking is dependent on conformation, wherein high titers of particles provided herein or of binding domains thereof result in fewer small ICs.

In some embodiments, the particle can be formatted to provide the optimal ratio of antigen to binding domain. At an optimal ratio, stable IC are generated at higher volume to ligate an increasing number of Fc family receptors. Methods of determining optimal ratio of antigen to binding domain, such as the ratio of any of the provided binding domains and a viral protein, are known in the art. Ratio of antigen to antigen binding domain (Ag:BD) can vary with epitope and includes 1:1, 1:5, 1:10, 1:20, 1:100, 1:500, 5:1, 10:1, 20:1, 100:1, 500:1. Such methods include determining experimental titer of binding agents, functional in vitro and in vivo studies of subsequent Fc family receptor ligation, generation of dose response curves, X-ray crystallography, and mass spectrometry.

In some embodiments, the particle comprises a viral protein capable of being bound by the at least one binding domain of the binding agent. In some embodiments, the viral protein is a surface protein of a virus, such as any described herein. In some embodiments, the viral protein is an isolated or purified viral protein. In some embodiments, the viral protein is a recombinant or synthetic protein.

In some embodiments, the viral protein is the S (spike) glycoprotein of a SARS virus. In some aspects, the mature S protein is first synthesized as a single precursor polypeptide, also known as S(0), such as is set forth in SEQ ID NOs. 43 or 45. Upon proteolytic processing, the S polypeptide yields two subunits: S1, such as is set forth in amino acids 13-685 of SEQ IN NO: 43, and S2, such as is set forth in amino acids 686-1273 or 816-1273 of SEQ ID NO. 43. The S1 subunit dominantly participates in receptor-virus interaction as it contains the surface exposed RBD. In some aspects, viral receptor interaction is mediated by the affinity of the RBD for angiotensin-converting enzyme 2 (ACE2) receptor on the surface of host cells. In some aspects, antibodies developed to SARS coronavirus have affinity for the RBD. Upon interaction of the RBD and receptor, the S2 subunit undergoes conformational change which exposes an internal hydrophobic fusion loop that facilitates membrane fusion between the viral and host membranes.

In some aspects, the viral protein is a recombinant S protein. In some aspects, the recombinant protein is of the S polypeptide precursor, S(0), such as is set forth in SEQ ID NO. 43. In some aspects, the recombinant protein is of the S1 subunit, such as is set forth in amino acids 13-685 of SEQ IN NO: 43. In some aspects, the recombinant protein is the RBD. In some aspects, the RBD is 331 to 524 of S protein or SEQ ID NO. 43.

In some aspects, the viral protein is the S (spike) glycoprotein of a SARS virus variant. In some aspects, the viral protein has one or more mutations in comparison to the wildtype sequence as set forth in SEQ ID NOs. 43 or 45. In some aspects, the one or more mutations is selected from the group comprising: D614G (also known as “Doug”), N501Y (also known as “Nelly”), P681H (also known as “Pooh”), and/or E484K (also known as “Eeek”). In some aspects, the viral protein is the S glycoprotein of a SARS virus variant belonging to the lineage selected from the group comprising B.1.1.7, B.1.351, and/or B.1.1.248. In some aspects, the viral protein is the S glycoprotein of a SARS virus variant classified as 501Y.V1, 501.V2, N501Y.V2 and/or P1.

TABLE 3
Exemplary SARS CoV-2 Natural Variants

Deletions (Δ) and Substitutions (μ) with
respect to SEQ ID NO. 43

Alpha

| | |
|---|---|
| B.1.1.7 | Δ69-70, 144; μN501Y (Nelly), μA570D, μD614G (Doug), |
| | μP681H (Pooh), μT716I, μS982A, μD1118H |

Beta

| | |
|---|---|
| B.1.351 | μL18F, μD80A, μD215G, μK417N, μE484K (Eek), |
| | μN501Y (Nelly), μD614G (Doug), μA701V |

Delta

| | |
|---|---|
| B.1.617.2 | Δ156-157; μG142D, μR158G, μL452R, μT478K, μD614G |
| | (Doug), μP681R, μD950N |
| AY.1 | Δ156-157; μG142D, μR158G, μK417N, μL452R, μT478K, |
| (Delta Plus) | μD614G (Doug), μP681R, μD950N |

Gamma

| | |
|---|---|
| P.1 | μL18F, μT20N, μP26S, μD138Y, μR190S, μK417N/T, |
| | μE484K (Eek), μN501Y (Nelly), μD614G (Doug), |
| | μH655Y, μT1027I, μG1219V |

In some aspects, the viral protein is the S glycoprotein of a variant of SARS CoV-2. Exemplary SARS CoV-2 variants are shown above in Table 3. In some aspects, the viral protein is the S glycoprotein of a SARS CoV-2 Variant of Interest (Vol), Variant of Concern (VoC), and/or Variant of High Consequence (VoHC). In some aspects, the SARS CoV-2 variant is chosen from the group comprising: Alpha (i.e., B.1.1.7), Beta (i.e., B.1.351, B.1.351.2, B.1.351.3), Delta (i.e., B.1.617.2, AY.1, AY.2, AY.3), and Gamma (i.e., P.1, P.1.1, P.1.2). In some aspects, the viral S protein of a SARS CoV-2 variant (i.e., any of a Vol, VoC, or VoHC) has one or more mutations in comparison to the wildtype S sequence as set forth in SEQ ID NOs. 43 or 45.

### II. Vehicles and Methods for Delivery of Binding Agents

Provided herein are vehicles containing the provided binding agent polypeptides. Also provided herein are polynucleotides encoding the binding agent. In some embodiments, a binding agent, or particle, is introduced to a cell in the subject. Also provided are methods of delivering any of the provided binding agents or particles, or polynucleotides encoding the binding agent, to a cell infected with a virus, such as a coronavirus. In some embodiments, the polynucleotides can be administered as a naked nucleic acid (e.g. mRNA or DNA) or can be delivered in a carrier or vehicle for delivery to the subject. In some embodiments, the binding agent polypeptide can be administered as a recombinant or purified molecule or can be delivered in a carrier or vehicle for delivery to the subject. In some embodiments, the particle polypeptide is administered as a complex, containing the at least one binding agent and a viral protein bound by the at least one binding domain of the binding agent.

In some embodiments, a polynucleotide encoding a binding agent is contained in a vehicle, such as viral-particles, viral-like particles, or non-viral particles, for delivering the polynucleotides to the subject.

In some embodiments, the polynucleotide is delivered as a naked nucleic acid. In some embodiments, the polynucleotide is administered as an mRNA. In some embodiments, the polynucleotide is administered as DNA, e.g., a plasmid.

Polynucleotides encoding a binding agent of the present invention may be delivered to a cell naked. As used herein in, “naked” refers to delivering a binding agent free from agents which promote internalization. For example, the polynucleotide delivered to the cell may contain no modifications. The naked polynucleotides may be delivered to the cell using routes of administration known in the art and described herein.

In some aspects, mRNAs may be delivered as packaged particles (e.g., encapsulated in a delivery vehicle) or unpackaged (i.e., naked). In some aspects, mRNA may be transcribed within host cells. Exogenous mRNA delivery was first investigated in 1990, wherein Wolff and colleagues observed protein expression in mice following injection of mRNA encoding a reporter gene (Wolff et al., Science (247) 1465, 1990). Once exogenous mRNA has been transmitted to the cytosol, in some aspects, host cellular machinery can produce a mature polypeptide. In some embodiments, the polypeptide can be subject to post-translational modifications. In some aspects, proteins produced from exogenous mRNA delivery are degraded by normal physiological processes. In some embodiments, mRNA delivery reduces risk of metabolite toxicity (Pardi et al., Nat Rev Drug Discov (17) 4, 2018).

According to the provided embodiments, polynucleotides administered mRNA may have a capping region. The capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

Wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. In some aspects, the in vivo half-life of mRNA can be regulated via modifications to the 3′ poly-adenosine tail. The specific features can also be incorporated to ensure controlled down-regulation of the transcript in case they are misdirected to undesired organs sites.

In some embodiments, the polynucleotide encoding a binding agent is operably linked to a promoter to control expression. In some embodiments, promoter elements regulate the frequency of transcriptional initiation. A promoter may be one naturally associated with a gene or polynucleotide sequence, as may be obtained by isolating the 5′ non-coding sequences located upstream of the coding segment and/or exon. Such a promoter can be referred to as “endogenous.” Alternatively, certain advantages will be gained by positioning the coding polynucleotide segment under the control of a recombinant or heterologous promoter, which refers to a promoter that is not normally associated with a polynucleotide sequence in its natural environment. Such promoters may include promoters of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence or a shortened Cytomegalovirus immediate early (CMVie) promoter (Ostedgaard et al., Proc. Natl. Acad. Sci. USA 2005, 102, 2952-2957). In some embodiments, the promoter sequence is a strong constitutive promoter sequence capable of driving high levels of expression of any polynucleotide sequence operatively linked thereto. In some embodiments, a suitable promoter is Elongation Growth Factor-1a (EF-1 a). In some embodiments, other constitutive promoter sequences may also be used, including, but not limited to the simian virus 40 (SV40) early promoter, silencing-prone spleen focus forming virus (SFFV) promoter, silencing-prone spleen focus forming virus (SFFV) promoter, mouse mammary tumor virus (MMTV), human immunodeficiency virus (HIV) long terminal repeat (LTR) promoter, MoMuLV promoter, an avian leukemia virus promoter, a CAG promoter (Halbert et al., Hum. Gene Ther. 2007, 18, 344-354), an Epstein-Barr virus immediate early promoter, a Rous sarcoma virus promoter, a F5tg83 promoter (Yan et al., 2015, Hum. Gene Ther., 26:334-346), as well as human gene promoters such as, but not limited to, the actin promoter, the myosin promoter, the hemoglobin promoter, and the creatine kinase promoter, and hybrid promoters, such as a hybrid promoter having a human cytomegalovirus (CMV) enhancer and the elongation factor 1a promoter (hCEF).

In some embodiments, the promoter is a constitutive promoter. In some aspects, a constitutive promoters may be a ubiquitous promoter that allows expression in a wide variety of cell and tissue types. In some embodiments, the promoter is a human Ubiquitin C (UbC) promoter, a human elongation factor 1a (EFla) promoter, an SV40 promoter, a Cytomegalovirus (CMV) promoter, or a PGK-1 promoter.

In some embodiments, any of the provided polynucleotides encoding a binding agent can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In provided embodiments, the methods for treating a viral infection (e.g. a coronavirus infection), include delivering a binding agent or particle protein to a subject. In some embodiments, the protein can be administered as a recombinant or purified protein. In some embodiments, the protein can be delivered in a carrier or vehicle for delivery to the subject. In provided embodiments, the binding agent or particle protein can have the amino acid sequence of any of a binding agent polypeptide as described above.

In some embodiments, the protein is produced using recombinant DNA techniques. In some embodiments, a nucleic acid molecule is an expression vector that is suitable for expression in a selected host cell. In some embodiments, the protein can be produced by gene synthesis methods

In some embodiments, a nucleic acid encoding a binding agent may be contained in an expression vector. Vectors comprising nucleic acids that encode a binding agent or polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In particular, a DNA vector that encodes a binding agent can be can be used to facilitate the expression and recombinant production of a binding agent. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. The methods producing a binding agent polypeptide may include culturing a cell under conditions that lead to expression of the polypeptide, wherein the cell comprises a nucleic acid molecule encoding a binding agent described herein, and/or vectors that include these nucleic acid sequences. In some embodiments, a binding agent or may be expressed in prokaryotic cells, such as bacterial cells; or in eukaryotic cells, such as fungal cells (such as yeast), plant cells, insect cells, and mammalian cells. In some of any of the provided embodiments, the binding agent is administered as an agent for delivery to a cell in a subject, such as a subject that is known or is likely to be infected with a virus (e.g. a coronavirus). In some embodiments, a binding agent or particle is contained in a vehicle, such as viral-particles, viral-like particles, or non-viral particles, for delivering the protein to the subject. Exemplary vehicles for delivery are described in Section II.

In some embodiments, the vehicle is a viral vector or is derived from a viral vector. In other embodiments, the vehicle is a non-viral vector, such as a cellular particle, liposome, nanoparticle, or other synthetic particle.

Non-viral vectors and methods employing the use of polymers, surfactants, and/or excipients have been employed to introduce polynucleotides and polypeptides into cells including conjugation with a targeting moiety, conjugation with a cell penetrating peptide, derivatization with a lipid and incorporation into liposomes, lipid nanoparticles, and cationic liposomes. The majority of non-viral vectors consist of plasmid DNA complexed with lipids or polycations. Many different lipids with ability to deliver plasmid DNA to cells in vitro and in vivo have been reported (Gao, et al., Gene Therapy 2:710-722 (1995)).

In particular embodiments, the polynucleotide or polypeptide is encapsulated within the lumen of a lipid particle in which the lipid particle contains a lipid bilayer, a lumen surrounded by the lipid bilayer. In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

A. Viral Vectors

Provided herein are vehicles containing a binding agent or particle, such as any of the polynucleotides or polypeptides described in Section I, that are derived from virus, such as viral particles. In some embodiment the viral particles include those derived from retroviruses or lentiviruses. In some embodiments, the viral particle's bilayer of amphipathic lipids is or comprises the viral envelope. In some embodiments, the viral particle's bilayer of amphipathic lipids is or comprises lipids derived from an infected host cell.

Biological methods for introducing an exogenous agent to a host cell include the use of DNA and RNA vectors. DNA and RNA vectors can also be used to house and deliver polynucleotides and polypeptides. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the polynucleotides and polypeptides are comprised within a viral vector. In some embodiments, the polynucleotides or polypeptides provided herein (e.g., a binding agent or particle) are administered host cells using recombinant virus particles, such as, e.g., vectors derived from adenoviruses and adeno-associated virus (AAV).

In some embodiments, the AAV vector is of serotype 1, 2, 6, 8 or 9. In some embodiments, the AAV vector is of serotype 6.2.

In some embodiments, the AAV vector includes a capsid that is a chimera between AAV2 (aa 1-128) and AAV5 (aa 129-725) with one point mutation (A581T) (AAV2.5T, Excoffon et al. Proc Natl Acad Sci. 106(10):3875-70, 2009).

The AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3.

In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells.

In some embodiment, suitable host cells for producing AAV derived vehicles include microorganisms, yeast cells, insect cells, and mammalian cells. In some embodiments, the term host cell includes the progeny of the original cell which has been transfected. Thus, as indicated above, a “host cell,” or “producer cell,” as used herein, generally refers to a cell which has been transfected with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral Ela and Elb genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences, such as those encoding a binding agent or particle as provided in Sections I, II, and III. In some embodiments, producer cells yield AAV virions. AAV helper functions are generally AAV-derived coding sequences that may be expressed to provide AAV gene products that, in turn, function for productive AAV replication. In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting the host cell with a mixture of AAV helper constructs either prior to, or concurrently with, the transfection of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

In some embodiments, the virus particles are lentivirus. In some embodiments, the lentiviral vector particle is Human Immunodeficiency Virus-1 (HIV-1).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740)

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003.

A number of preclinical studies have demonstrated therapeutic and prophylactic efficacy of viral vector based gene delivery in animal models and in clinical trials. In some aspects, viral and virally derived vectors capable of replication provide consistent gene expression over time. In some aspects, replication competent viruses can result in undesired immunogenicity, toxicity, and cell death. In some embodiments, vectors capable of insertion are efficient for transduction of a variety of cells. However, in some aspects, they can pose a risk of insertional mutagenesis. Integration-deficient vectors can persist episomally but can also retain the transduction efficiency of standard integrating vectors. Thus, in some embodiments, the vector particle is replication deficient. In some embodiments, the vector particle is integration deficient. Various methods of rendering a vector insertional or replication deficient are known in the art. Various replication-defective vaccine vectors have been produced with many other viruses, including adeno-associated virus (AAV), poliovirus, and Sendai virus.

B. Virus-Like Particles

Also provided herein are virus-like particles (VLP) containing a binding agent or particle, such as any of the polynucleotides or polypeptides provided herein. The VLPS include those derived from retroviruses or lentiviruses. While VLPs mimic native virion structure, they lack the viral genomic information necessary for independent replication within a host cell. Therefore, in some aspects, VLPs are non-infectious. In some embodiments, the VLP's bilayer of amphipathic lipids is or comprises the viral envelope. In some embodiments, the targeted lipid particle's bilayer of amphipathic lipids is or comprises lipids derived from a cell. A VLP typically comprises at least one type of structural protein from a virus. In most cases this protein will form a proteinaceous capsid (e.g. VLPs comprising a lentivirus, adenovirus or paramyxovirus structural protein). In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

Provided herein are VLPs that are derived from virus, such as those derived from retroviruses or lentiviruses. In some embodiments, the viral particles are derived from paramyxoviruses. Thus, in some examples, the viral-like particle is derived from Nipah, Hendra, or Rubeola viruses.

C. Non-Viral Vectors

In some embodiments, the polynucleotides or polypeptides provided herein are not comprised within a viral or virally derived vector. Provided herein are non-viral vectors containing a binding agent or particle, such as any of the polynucleotides or polypeptides described herein. In some embodiments, the binding agent or particle peptide or encoding polynucleotide is comprised within a non-viral vector or delivery vehicle. In some embodiments, the binding agent or particle encoding polynucleotide is comprised within a non-viral vector or delivery vehicle.

Among provided non-viral vectors are lipid particles. In some embodiments, the lipid particle comprises a naturally derived bilayer of amphipathic lipids. In some embodiments, the bilayer may be comprised of one or more lipids of the same or different type. In some embodiments, the lipids comprise phospholipids such as phosphocholines and phosphoinositols. In some embodiments, the lipids comprise DMPC, DOPC, and DSPC. In some embodiments, the polynucleotides or polypeptides provided herein are comprised in a non-viral vector, such as a lipid particle.

Nanoparticles are solid, spherical structures ranging to about 100 nm in size and can be prepared from natural or synthetic polymers. In some aspects, nanoparticles display the ability to target specific tissues or cells, protect target genes against nuclease degradation, improve DNA stability, and increase transformation efficiency or safety. In some embodiments, the non-viral vector is a nanoparticle. In some embodiments, the nanoparticle is one or more of any of biodegradable polymer, tetrapod quantum dot, tetrapod article, multi-legged luminescent nanoparticle, tetrapod nanocrystal, biodegradable nanoparticle, liposome, nanocarrier, or dendrimer.

Cationic lipids are amphiphilic molecules that have a cationic head group and a hydrophobic tail group connected by either stable or degradable linkages. Felgner and colleagues were the first to demonstrate the use of cationic lipids for DNA delivery in 1987 (Felgner et al. PNAS (84) 21:7413-7417, 1987). Many cationic lipids since then have been synthesized and evaluated for nucleic acid delivery, including for example GL67A. Thus, in some embodiments, the non-viral vector is a lipid complex. In some embodiments, the non-viral vector is a plasmid. In some embodiments, the non-viral vector is naked nucleic acid.

In some embodiments, the vector is mRNA. Non-viral delivery of mRNA can be obtained using injection of naked nucleic acid, polyplex, lipoplex or liposome-encapsulated mRNA, biolistic delivery by gene gun, microparticle carrier mediated delivery, and electroporation.

In some aspects, the basic components of an mRNA molecule include at least a coding region, a 5′UTR, a 3′UTR, a 5′ cap and a poly-A tail. In some embodiments, the first and second flanking regions range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides). In some embodiments, the tailing sequence ranges from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In some embodiments, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

The 5′ cap structure of an mRNA is involved in nuclear export, increasing mRNA stability. In some aspects, the 5′ cap binds the mRNA Cap Binding Protein (CBP), which in turn associates with poly(A) binding protein to form the mature cyclic mRNA species. In some aspects, the cap further assists the removal of 5′ proximal introns removal during mRNA splicing. Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may also be 2′-O-methylated. 5′-decapping through hydrolysis and cleavage of the guanylate cap structure may target a nucleic acid molecule, such as an mRNA molecule, for degradation.

In some embodiments, modifications to the polynucleotides provided herein, such as an mRNA vector encoding a binding agent, may generate a non-hydrolyzable cap structure preventing decapping and thus increasing mRNA half-life. Because cap structure hydrolysis requires cleavage of 5′-ppp-5′ phosphorodiester linkages, in some aspects modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

In some embodiments, additional modified guanosine nucleotides may be used such as a-methyl-phosphonate and seleno-phosphate nucleotides. In some aspects, additional modifications include 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, also referred to as synthetic cap analogs, chemical caps, chemical cap analogs, or structural or functional cap analogs, differ from natural (i.e. endogenous, wild-type or physiological) 5′-caps in their chemical structure, while retaining cap function. In some aspects, cap analogs may be chemically (i.e. non-enzymatically) or enzymatically synthesized and/or linked to a nucleic acid molecule.

In some embodiments, the capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

In some aspects, the polynucleotides provided herein, such as a non-viral mRNA vector, comprises a region of polynucleic acid sequence that is partially or substantially not translatable, e.g., having a noncoding region. Such molecules are generally not translated, but can exert an effect on protein production by one or more of binding to and sequestering one or more translational machinery components such as a ribosomal protein or a transfer R A (tR A), thereby effectively reducing protein expression in the cell or modulating one or more pathways or cascades in a cell which in turn alters protein levels. In some aspects, the polynucleotides provided herein contain a noncoding region. In some embodiments, the noncoding region encodes one or more long noncoding RNA (lncRNA, or lincRNA) or portion thereof, a small nucleolar RNA (sno-RNA), micro RNA (miRNA), small interfering RNA (siRNA) or Piwi-interacting RNA (piRNA).

In some embodiments, modification of 3′ untranslated region AU rich elements (AREs) is used to modulate the stability of polynucleotides. When engineering specific polynucleotides, such as those encoding a binding agent or particle, one or more copies of an ARE can be introduced to make polynucleotides provided herein less stable. In some aspects, reducing stability of the polynucleotide reduces cognate protein expression. In some aspects, AREs can be identified and removed or mutated to increase the intracellular stability and thus increase translation and production of the resultant protein. Transfection experiments can be conducted in relevant cell lines, using polynucleotides provided herein and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

In some embodiments, the polynucleotides provided herein may further comprise, in addition to a Start and/or Stop codon, one or more signal and/or restriction sequences.

In some aspects, non-viral nucleic acid transfer or delivery vehicles can be less toxic, less antigenic, easier, and less expensive to prepare than viral vectors for delivery of nucleic acids. Certain delivery vehicles, such as cationic lipid or polymer delivery vehicles, can also help protect from endogenous RNAase during nucleic acid transfer.

D. Methods of Generating Lipid Particles

Provided herein is a lipid particle comprising a binding agent or particle, such as any of the polynucleotides or polypeptides described herein. In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

In some embodiments, lipid particles may be produced in multiple cell culture systems including bacteria, mammalian cell lines, insect cell lines, yeast and plant cells.

In some embodiments, the assembly of a lipid particle is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the vehicle is a targeted lipid particle which comprises a sequence that is devoid of or lacking viral RNA, which in some aspects may be the result of removing or eliminating the viral RNA from the sequence. In some embodiments, this may be achieved by using an endogenous packaging signal binding site on gag. In some embodiments, the endogenous packaging signal binding site is on pol. In some embodiments, the polynucleotides provided herein, such as those encoding any of the provided binding agents, will contain a cognate packaging signal. In some embodiments, a heterologous binding domain (which is heterologous to gag) located on the polynucleotides provided herein to be delivered, and a cognate binding site located on gag or pol, can be used to ensure packaging of the polynucleotides provided herein to be delivered. In some embodiments, the vector particles could be used to deliver the polynucleotides or polypeptides provided herein, in which case functional integrase and/or reverse transcriptase is not required.

1. Transfer Vectors

In some embodiments, a vector particle comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. In some aspects, vector particles will typically include various viral components and sometimes also host cell components in addition to nucleic acid(s). In some embodiments, a vector comprises e.g., a virus or viral particle capable of transferring a nucleic acid into a cell, or to the transferred nucleic acid (e.g., as naked mRNA). In some embodiments, viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

2 Packaging Vectors

Large scale vector particle production is often useful to achieve a desired concentration of vector particles. Particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a producer cell, and are introduced into the cell via transfection, transduction or infection. A retroviral, e.g., lentiviral, transfer vector can be introduced into a producer cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

E. Vehicle Targeting and Retargeting

In some embodiments, the vehicle further comprises a vector-surface targeting moiety which specifically binds to a target ligand. It will be recognized by those skilled in the art that, the vehicles provided herein harbor the attachment and/or fusion glycoproteins and are capable of binding to target cells and delivering the vehicle contents to the cytoplasm of the target cells. It will also be recognized by those skilled in the art that this is due to the natural viral entry mechanism that involves fusion of the viral membrane directly with the target cell plasma membrane.

It will further be recognized by those skilled in the art that many viruses such as paramyxoviruses bind to sialic acid receptors, and hence the corresponding derivative vehicles can deliver their contents generically to nearly any kind of cell that expresses sialic acid receptors. Other viruses such as Nipah virus and HIV bind to protein receptors, and hence the corresponding vehicles have a specificity that matches the natural tropisms for each virus and its surface proteins.

Furthermore, it will be recognized that technology exists to “re-target” attachment proteins, making it so that the vehicles only interact with particular cells or cell types that express a marker protein of interest (Msaouel et al., Meths Mol Biol 797: 141-162, 2012). Thus, vehicle surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

1. Fusogens

In some embodiments, the provided vehicles, e.g. lipid particles, such as viral vectors or viral-like particles, contain one or more fusogens. In some embodiments, the lipid particle, e.g. viral vector or viral-like particle, contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane. In some embodiments, the lipid particle, such as a viral or non-viral vector, comprising the fusogen integrates into the membrane into a lipid bilayer of a target cell. In some embodiments, the fusogen results in mixing between lipids in the lipid particle and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the lipid particle, e.g. viral vector or viral-like particle, contain a first fusogen that is a protein fusogen and a second fusogen that is a lipid fusogen or chemical fusogen. In some embodiments, the fusogen binds a fusogen binding partner on a target cell surface. In some embodiments, the lipid particle is a viral vector or viral-like particle that is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gpl60 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of 0-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and R sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the sequence of the F protein is as set forth in SEQ ID. NO 46. In some embodiments, the F protein is truncated and lacks up to 42 contiguous amino acids, such as up to 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the C-terminus of SEQ ID. NO 46.

In some embodiments, the sequence of the HN protein is as set forth in SEQ ID. NO 47. In some embodiments, the HN protein is modified, such as by modification to the C-terminal domain. In some embodiments, the sequence of the HN protein is as set forth in SEQ ID. NO 48.

In some embodiments, the fusogen is a F and/or HN protein from the murine parainfluenza virus type 1 (See e.g., U.S. patent Ser. No. 10/704,061).

a. G Proteins

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein or a biologically active portion thereof. A non-limited list of exemplary G proteins is shown in Table 4A.

The attachment G proteins are type II transmembrane glycoproteins containing an N-terminal cytoplasmic tail (e.g. corresponding to amino acids 1-49 of SEQ ID NO:5), a transmembrane domain (e.g. corresponding to amino acids 50-70 of SEQ ID NO:5, and an extracellular domain containing an extracellular stalk (e.g. corresponding to amino acids 71-187 of SEQ ID NO:5), and a globular head (corresponding to amino acids 188-602 of SEQ ID NO:5). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

TABLE 4A
Exemplary Henipavirus G Proteins

| | | | SEQ ID |
|---|---|---|---|
| | | | NO |
| | | | (with- |
| | | | out N- |
| | | | termi- |
| Viral | | SEQ | nal |
| G | | ID | methi- |
| Protein | Sequence | NO | onine) |
| Hendra | MMADSKLVSLNNNLSGKIKDQGKVIKNYYG | 6 | 7 |
| Virus | TMDIKKINDGLLDSKILGAFNTVIALLGSI | | |
| G | IIIVMNIMIIQNYTRTTDNQALIKESLQSV | | |
| Protein | QQQIKALTDKIGTEIGPKVSLIDTSSTITI | | |
| | PANIGLLGSKISQSTSSINENVNDKCKFTL | | |
| | PPLKIHECNISCPNPLPFREYRPISQGVSD | | |
| | LVGLPNQICLQKTTSTILKPRLISYTLPIN | | |
| | TREGVCITDPLLAVDNGFFAYSHLEKIGSC | | |
| | TRGIAKQRIIGVGEVLDRGDKVPSMFMTNV | | |
| | WTPPNPSTIHHCSSTYHEDFYYTLCAVSHV | | |
| | GDPILNSTSWTESLSLIRLAVRPKSDSGDY | | |
| | NQKYIAITKVERGKYDKVMPYGPSGIKQGD | | |
| | TLYFPAVGFLPRTEFQYNDSNCPIIHCKYS | | |
| | KAENCRLSMGVNSKSHYILRSGLLKYNLSL | | |
| | GGDIILQFIEIADNRLTIGSPSKIYNSLGQ | | |
| | PVFYQASYSWDTMIKLGDVDTVDPLRVQWR | | |
| | NNSVISRPGQSQCPRFNVCPEVCWEGTYND | | |
| | AFLIDRLNWVSAGVYLNSNQTAENPVFAVF | | |
| | KDNEILYQVPLAEDDTNAQKTITDCFLLEN | | |
| | VIWCISLVEIYDTGDSVIRPKLFAVKIPAQ | | |
| | CSES | | |
| Nipah | MPAENKKVRFENTTSDKGKIPSKVIKSYYG | 8 | 9 |
| Virus | TMDIKKINEGLLDSKILSAFNTVIALLGSI | | |
| G | VIIVMNIMIIQNYTRSTDNQAVIKDALQGI | | |
| Protein | QQQIKGLADKIGTEIGPKVSLIDTSSTITI | | |
| | PANIGLLGSKISQSTASINENVNEKCKFTL | | |
| | PPLKIHECNISCPNPLPFREYRPQTEGVSN | | |
| | LVGLPNNICLQKTSNQILKPKLISYTLPVV | | |
| | GQSGTCITDPLLAMDEGYFAYSHLERIGSC | | |
| | SRGVSKQRIIGVGEVLDRGDEVPSLFMTNV | | |
| | WTPPNPNTVYHCSAVYNNEFYYVLCAVSTV | | |
| | GDPILNSTYWSGSLMMTRLAVKPKSNGGGY | | |
| | NQHQLALRSIEKGRYDKVMPYGPSGIKQGD | | |
| | TLYFPAVGFLVRTEFKYNDSNCPITKCQYS | | |
| | KPENCRLSMGIRPNSHYILRSGLLKYNLSD | | |
| | GENPKVVFIEISDQRLSIGSPSKIYDSLGQ | | |
| | PVFYQASFSWDTMIKFGDVLTVNPLVVNWR | | |
| | NNTVISRPGQSQCPRFNTCPEICWEGVYND | | |
| | AFLIDRINWISAGVFLDSNQTAENPVFTVF | | |
| | KDNEILYRAQLASEDTNAQKTITNCFLLKN | | |
| | KIWCISLVEIYDTGDNVIRPKLFAVKIPEQ | | |
| | CT | | |
| Cedar | MLSQLQKNYLDNSNQQGDKMNNPDKKLSVN | 10 | 11 |
| Virus | FNPLELDKGQKDLNKSYYVKNKNYNVSNLL | | |
| G | NESLHDIKFCIYCIFSLLIIITIINIITIS | | |
| Protein | IVITRLKVHEENNGMESPNLQSIQDSLSSL | | |
| | TNMINTEITPRIGILVTATSVTLSSSINYV | | |
| | GTKTNQLVNELKDYITKSCGFKVPELKLHE | | |
| | CNISCADPKISKSAMYSTNAYAELAGPPKI | | |
| | FCKSVSKDPDFRLKQIDYVIPVQQDRSICM | | |
| | NNPLLDISDGFFTYIHYEGINSCKKSDSFK | | |
| | VLLSHGEIVDRGDYRPSLYLLSSHYHPYSM | | |
| | QVINCVPVTCNQSSFVFCHISNNTKTLDNS | | |
| | DYSSDEYYITYFNGIDRPKTKKIPINNMTA | | |
| | DNRYIHFTFSGGGGVCLGEEFIIPVTTVIN | | |
| | TDVFTHDYCESFNCSVQTGKSLKEICSESL | | |
| | RSPTNSSRYNLNGIMIISQNNMTDFKIQLN | | |
| | GITYNKLSFGSPGRLSKTLGQVLYYQSSMS | | |
| | WDTYLKAGFVEKWKPFTPNWMNNTVISRPN | | |
| | QGNCPRYHKCPEICYGGTYNDIAPLDLGKD | | |
| | MYVSVILDSDQLAENPEITVFNSTTILYKE | | |
| | RVSKDELNTRSTTTSCFLFLDEPWCISVLE | | |
| | TNRFNGKSIRPEIYSYKIPKYC | | |
| Bat | MPQKTVEFINMNSPLERGVSTLSDKKTLNQ | 12 | 13 |
| Paramy- | SKITKQGYFGLGSHSERNWKKQKNQNDHYM | | |
| xovirus | TVSTMILEILVVLGIMFNLIVLTMVYYQND | | |
| G | NINQRMAELTSNITVLNLNLNQLTNKIQRE | | |
| Protein, | IIPRITLIDTATTITIPSAITYILATLTTR | | |
| Eid_hel/ | ISELLPSINQKCEFKTPTLVLNDCRINCTP | | |
| GH- | PLNPSDGVKMSSLATNLVAHGPSPCRNFSS | | |
| M74a/ | VPTIYYYRIPGLYNRTALDERCILNPRLTI | | |
| GHA/2009 | SSTKFAYVHSEYDKNCTRGFKYYELMTFGE | | |
| | ILEGPEKEPRMFSRSFYSPTNAVNYHSCTP | | |
| | IVTVNEGYFLCLECTSSDPLYKANLSNSTF | | |
| | HLVILRHNKDEKIVSMPSFNLSTDQEYVQI | | |
| | IPAEGGGTAESGNLYFPCIGRLLHKRVTHP | | |
| | LCKKSNCSRTDDESCLKSYYNQGSPQHQVV | | |
| | NCLIRIRNAQRDNPTWDVITVDLTNTYPGS | | |
| | RSRIFGSFSKPMLYQSSVSWHTLLQVAEIT | | |
| | DLDKYQLDWLDTPYISRPGGSECPFGNYCP | | |
| | TVCWEGTYNDVYSLTPNNDLFVTVYLKSEQ | | |
| | VAENPYFAIFSRDQILKEFPLDAWISSART | | |
| | TTISCFMFNNEIWCIAALEITRLNDDIIRP | | |
| | IYYSFWLPTDCRTPYPHTGKMTRVPLRSTY | | |
| | NY | | |
| Mojiang | MATNRDNTITSAEVSQEDKVKKYYGVETAE | 14 | 15 |
| virus, | KVADSISGNKVFILMNTLLILTGAIITITL | | |
| Tongguan | NITNLTAAKSQQNMLKIIQDDVNAKLEMFV | | |
| 1 G | NLDQLVKGEIKPKVSLINTAVSVSIPGQIS | | |
| Protein | NLQTKFLQKYVYLEESITKQCTCNPLSGIF | | |
| | PTSGPTYPPTDKPDDDTTDDDKVDTTIKPI | | |
| | EYPKPDGCNRTGDHFTMEPGANFYTVPNLG | | |
| | PASSNSDECYTNPSFSIGSSIYMFSQEIRK | | |
| | TDCTAGEILSIQIVLGRIVDKGQQGPQASP | | |
| | LLVWAVPNPKIINSCAVAAGDEMGWVLCSV | | |
| | TLTAASGEPIPHMFDGFWLYKLEPDTEVVS | | |
| | YRITGYAYLLDKQYDSVFIGKGGGIQKGND | | |
| | LYFQMYGLSRNRQSFKALCEHGSCLGTGGG | | |
| | GYQVLCDRAVMSFGSEESLITNAYLKVNDL | | |
| | ASGKPVIIGQTFPPSDSYKGSNGRMYTIGD | | |
| | KYGLYLAPSSWNRYLRFGITPDISVRSTTW | | |
| | LKSQDPIMKILSTCTNTDRDMCPEICNTRG | | |
| | YQDIFPLSEDSEYYTYIGITPNNGGTKNFV | | |
| | AVRDSDGHIASIDILQNYYSITSATISCFM | | |
| | YKDEIWCIAITEGKKQKDNPQRIYAHSYKI | | |
| | RQMCYNMKSATVTVGNAKNITIRRY | | |

In some embodiments, the G protein has a sequence set forth in any of SEQ ID NOS: 5-15 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 5, 6, 7, 8, 9, 10, 11, 12, 13, 14, or 15.

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F).

In particular embodiments, the G protein has the sequence of amino acids set forth in SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 and retains fusogenic activity in conjunction with a Henipavirus F protein (e.g., NiV-F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:5, SEQ ID NO:8 or SEQ ID NO:9, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:5, SEQ ID NO:8 or SEQ ID NO:9.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:5, SEQ ID NO:8 or SEQ ID NO:9), up to 6 contiguous amino acid residues at or near the N-terminus of the wild-type NiV-G protein (SEQ ID NO:5, SEQ ID NO:8 or SEQ ID NO:9), up to 7 contiguous amino acid residues at or near the N-terminus of the wild-type NiV-G protein (SEQ ID NO:5, SEQ ID NO:8 or SEQ ID NO:9), up to 8 contiguous amino acid residues at or near the N-terminus of the wild-type NiV-G protein (SEQ ID NO:5, SEQ ID NO:8 or SEQ ID NO:9), up to 9 contiguous amino acid residues at or near the N-terminus of the wild-type NiV-G protein (SEQ ID NO:5, SEQ ID NO:8 or SEQ ID NO:9) up to 10 contiguous amino acid residues at or near the N-terminus of the wild-type NiV-G protein (SEQ ID NO:5, SEQ ID NO:8 or SEQ ID NO:9), up to 11 contiguous amino acid residues at or near the N-terminus of the wild-type NiV-G protein (SEQ ID NO:5, SEQ ID NO:8 or SEQ ID NO:9), up to 12 contiguous amino acid residues at or near the N-terminus of the wild-type NiV-G protein SEQ ID NO:9, SEQ ID NO:5, SEQ ID NO:8 or SEQ ID NO:8), up to 13 contiguous amino acid residues at or near the N-terminus of the wild-type NiV-G protein (SEQ ID NO:5, SEQ ID NO:8 or SEQ ID NO:9), up to 14 contiguous amino acid residues at or near the N-terminus of the wild-type NiV-G protein (SEQ ID NO:5, SEQ ID NO:8 or SEQ ID NO:9), up to 15 contiguous amino acid residues at or near the N-terminus of the wild-type NiV-G protein (SEQ ID NO:5, SEQ ID NO:8 or SEQ ID NO:9) up to 16 contiguous amino acid residues at or near the N-terminus of the wild-type NiV-G protein (SEQ ID NO:5, SEQ ID NO:8 or SEQ ID NO:9), up to 17 contiguous amino acid residues at or near the N-terminus of the wild-type NiV-G protein (SEQ ID NO:5, SEQ ID NO:8 or SEQ ID NO:9), up to 18 contiguous amino acid residues at or near the N-terminus of the wild-type NiV-G protein (SEQ ID NO:5, SEQ ID NO:8 or SEQ ID NO:9), up to 19 contiguous amino acid residues at or near the N-terminus of the wild-type NiV-G protein (SEQ ID NO:5, SEQ ID NO:8 or SEQ ID NO:9), up to 20 contiguous amino acid residues at or near the N-terminus of the wild-type NiV-G protein (SEQ ID NO:5, SEQ ID NO:8 or SEQ ID NO:9), up to 21 contiguous amino acid residues at or near the N-terminus of the wild-type NiV-G protein SEQ ID NO:5, SEQ ID NO:8 or SEQ ID NO:9), up to 22 contiguous amino acid residues at or near the N-terminus of the wild-type NiV-G protein (SEQ ID NO:5, SEQ ID NO:8 or SEQ ID NO:9), up to 23 contiguous amino acid residues at or near the N-terminus of the wild-type NiV-G protein (SEQ ID NO:5, SEQ ID NO:8 or SEQ ID NO:9), up to 24 contiguous amino acid residues at or near the N-terminus of the wild-type NiV-G protein (SEQ ID NO:5, SEQ ID NO:8 or SEQ ID NO:9), up to 25 contiguous amino acid residues at or near the N-terminus of the wild-type NiV-G protein (SEQ ID NO:5, SEQ ID NO:8 or SEQ ID NO:9), up to 26 contiguous amino acid residues at or near the N-terminus of the wild-type NiV-G protein (SEQ ID NO:5, SEQ ID NO:8 or SEQ ID NO:9), up to 27 contiguous amino acid residues at or near the N-terminus of the wild-type NiV-G protein (SEQ ID NO:5, SEQ ID NO:8 or SEQ ID NO:9), up to 28 contiguous amino acid residues at or near the N-terminus of the wild-type NiV-G protein (SEQ ID NO:5, SEQ ID NO:8 or SEQ ID NO:9), up to 29 contiguous amino acid residues at or near the N-terminus of the wild-type NiV-G protein (SEQ ID NO:5, SEQ ID NO:8 or SEQ ID NO:9), up to 30 contiguous amino acid residues at or near the N-terminus of the wild-type NiV-G protein (SEQ ID NO:5, SEQ ID NO:8 or SEQ ID NO:9), up to 31 contiguous amino acid residues at or near the N-terminus of the wild-type NiV-G protein (SEQ ID NO:5, SEQ ID NO:8 or SEQ ID NO:9), up to 32 contiguous amino acid residues at or near the N-terminus of the wild-type NiV-G protein (SEQ ID NO:5, SEQ ID NO:8 or SEQ ID NO:9), up to 33 contiguous amino acid residues at or near the N-terminus of the wild-type NiV-G protein (SEQ ID NO:5, SEQ ID NO:8 or SEQ ID NO:9), up to 34 contiguous amino acid residues at or near the N-terminus of the wild-type NiV-G protein (SEQ ID NO:5, SEQ ID NO:8 or SEQ ID NO:9), up to 35 contiguous amino acid residues at or near the N-terminus of the wild-type NiV-G protein (SEQ ID NO:5, SEQ ID NO:8 or SEQ ID NO:9), up to 36 contiguous amino acid residues at or near the N-terminus of the wild-type NiV-G protein (SEQ ID NO:5, SEQ ID NO:8 or SEQ ID NO:9), up to 37 contiguous amino acid residues at or near the N-terminus of the wild-type NiV-G protein (SEQ ID NO:5, SEQ ID NO:8 or SEQ ID NO:9) up to 38 contiguous amino acid residues at or near the N-terminus of the wild-type NiV-G protein (SEQ ID NO:5, SEQ ID NO:8 or SEQ ID NO:9), up to 39 contiguous amino acid residues at or near the N-terminus of the wild-type NiV-G protein (SEQ ID NO:5, SEQ ID NO:8 or SEQ ID NO:9), up to 40 contiguous amino acid residues at or near the N-terminus of the wild-type NiV-G protein (SEQ ID NO:5, SEQ ID NO:8 or SEQ ID NO:9), up to 41 contiguous amino acid residues at or near the N-terminus of the wild-type NiV-G protein (SEQ ID NO:5, SEQ ID NO:8 or SEQ ID NO:9), up to 42 contiguous amino acid residues at or near the N-terminus of the wild-type NiV-G protein (SEQ ID NO:5, SEQ ID NO:8 or SEQ ID NO:9), up to 43 contiguous amino acid residues at or near the N-terminus of the wild-type NiV-G protein (SEQ ID NO:5, SEQ ID NO:8 or SEQ ID NO:9), up to 44 contiguous amino acid residues at or near the N-terminus of the wild-type NiV-G protein (SEQ ID NO:5, SEQ ID NO:8 or SEQ ID NO:9), or up to 45 contiguous amino acid residues at or near the N-terminus of the wild-type NiV-G protein (SEQ ID NO:5, SEQ ID NO:8 or SEQ ID NO:9).

In some embodiments, the NiV-G protein is a biologically active portion that does not contain a cytoplasmic domain. In some embodiments, the NiV-G protein without the cytoplasmic domain is encoded by SEQ ID NO: 39.

In some embodiments, the mutant NiV-G protein comprises a sequence set forth in any of SEQ ID NOS: 29-39, or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 29-39.

In some embodiments, the mutant NiV-G protein has a 5 amino acid truncation at or near the N-terminus of the wild-type NiV-G protein (SEQ ID NO:29, SEQ ID NO: 30 or SEQ ID NO:31), such as set forth in SEQ ID NO: 32 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:32 or such as set forth in SEQ ID NO: 33 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:33 or such as set forth in SEQ ID NO: 34 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:34. In some embodiments, the mutant NiV-G protein has a 10 amino acid truncation at or near the N-terminus of the wild-type NiV-G protein (SEQ ID NO:35, SEQ ID NO:36 or SEQ ID NO:37), such as set forth in SEQ ID NO: 35 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35, or such as set forth in SEQ ID NO: 36 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36 or such as set forth in SEQ ID NO: 37 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:37.

In some embodiments, the mutant NiV-G protein has a 15 amino acid truncation at or near the N-terminus of the wild-type NiV-G protein (SEQ ID NO:38, or SEQ ID NO:39), such as set forth in SEQ ID NO: 38 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:38 or such as set forth in SEQ ID NO: 39 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:39.

In some embodiments, the G protein is a mutant HeV-G protein that is a biologically active portion of a wild-type HeV-G. In some embodiments, the biologically active portion is an N-terminally truncated fragment.

In some embodiments, the mutant G protein is a mutant HeV-G protein that has the sequence set forth in SEQ ID NO:40 or 41, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:40 or 41.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:40 or 41), up to 6 contiguous amino acid residues at or near the N-terminus of the wild-type HeV-G protein (SEQ ID NO:40 or 41), up to 7 contiguous amino acid residues at or near the N-terminus of the wild-type HeV-G protein (SEQ ID NO:40 or 41 or up to 8 contiguous amino acid residues at or near the N-terminus of the wild-type HeV-G protein (SEQ ID NO:40 or 41), up to 9 contiguous amino acid residues at or near the N-terminus of the wild-type HeV-G protein (SEQ ID NO:40 or 41), up to 10 contiguous amino acid residues at or near the N-terminus of the wild-type HeV-G protein (SEQ ID NO:40 or 41), up to 11 contiguous amino acid residues at or near the N-terminus of the wild-type HeV-G protein (SEQ ID NO:40 or 41), up to 12 contiguous amino acid residues at or near the N-terminus of the wild-type HeV-G protein (SEQ ID NO:40 or 41), up to 13 contiguous amino acid residues at or near the N-terminus of the wild-type HeV-G protein (SEQ ID NO: 40 or 41), up to 14 contiguous amino acid residues at or near the N-terminus of the wild-type HeV-G protein (SEQ ID NO: 40 or 41), up to 15 contiguous amino acid residues at or near the N-terminus of the wild-type HeV-G protein (SEQ ID NO: 40 or 41), up to 16 contiguous amino acid residues at or near the N-terminus of the wild-type HeV-G protein (SEQ ID NO: 40 or 41), up to 17 contiguous amino acid residues at or near the N-terminus of the wild-type HeV-G protein (SEQ ID NO: 40 or 41), up to 18 contiguous amino acid residues at or near the N-terminus of the wild-type HeV-G protein (SEQ ID NO: 40 or 41), up to 19 contiguous amino acid residues at or near the N-terminus of the wild-type HeV-G protein (SEQ ID NO: 40 or 41), up to 20 contiguous amino acid residues at or near the N-terminus of the wild-type HeV-G protein (SEQ ID NO: 40 or 41), up to 21 contiguous amino acid residues at or near the N-terminus of the wild-type HeV-G protein (SEQ ID NO: 40 or 41), up to 22 contiguous amino acid residues at or near the N-terminus of the wild-type HeV-G protein (SEQ ID NO: 40 or 41), up to 23 contiguous amino acid residues at or near the N-terminus of the wild-type HeV-G protein (SEQ ID NO: 40 or 41), up to 24 contiguous amino acid residues at or near the N-terminus of the wild-type HeV-G protein (SEQ ID NO: 40 or 41), up to 25 contiguous amino acid residues at or near the N-terminus of the wild-type HeV-G protein (SEQ ID NO: 40 or 41), up to 26 contiguous amino acid residues at or near the N-terminus of the wild-type HeV-G protein (SEQ ID NO: 40 or 41), up to 27 contiguous amino acid residues at or near the N-terminus of the wild-type HeV-G protein (SEQ ID NO: 40 or 41), up to 28 contiguous amino acid residues at or near the N-terminus of the wild-type HeV-G protein (SEQ ID NO: 40 or 41), up to 29 contiguous amino acid residues at or near the N-terminus of the wild-type HeV-G protein (SEQ ID NO: 40 or 41), up to 30 contiguous amino acid residues at or near the N-terminus of the wild-type HeV-G protein (SEQ ID NO: 40 or 41), up to 31 contiguous amino acid residues at or near the N-terminus of the wild-type HeV-G protein (SEQ ID NO: 40 or 41), up to 32 contiguous amino acid residues at or near the N-terminus of the wild-type HeV-G protein (SEQ ID NO: 40 or 41), up to 33 contiguous amino acid residues at or near the N-terminus of the wild-type HeV-G protein (SEQ ID NO: 40 or 41), up to 34 contiguous amino acid residues at or near the N-terminus of the wild-type HeV-G protein (SEQ ID NO: 40 or 41), up to 35 contiguous amino acid residues at or near the N-terminus of the wild-type HeV-G protein (SEQ ID NO: 40 or 41), up to 36 contiguous amino acid residues at or near the N-terminus of the wild-type HeV-G protein (SEQ ID NO: 40 or 41), up to 37 contiguous amino acid residues at or near the N-terminus of the wild-type HeV-G protein (SEQ ID NO: 40 or 41), up to 38 contiguous amino acid residues at or near the N-terminus of the wild-type HeV-G protein (SEQ ID NO: 40 or 41), up to 39 contiguous amino acid residues at or near the N-terminus of the wild-type HeV-G protein (SEQ ID NO: 40 or 41), up to 40 contiguous amino acid residues at or near the N-terminus of the wild-type HeV-G protein (SEQ ID NO: 40 or 41), up to 41 contiguous amino acid residues at or near the N-terminus of the wild-type HeV-G protein (SEQ ID NO: 40 or 41), up to 42 contiguous amino acid residues at or near the N-terminus of the wild-type HeV-G protein (SEQ ID NO: 40 or 41), up to 43 contiguous amino acid residues at or near the N-terminus of the wild-type HeV-G protein (SEQ ID NO: 40 or 41), up to 44 contiguous amino acid residues at or near the N-terminus of the wild-type HeV-G protein (SEQ ID NO: 40 or 41), or up to 45 contiguous amino acid residues at or near the N-terminus of the wild-type HeV-G protein (SEQ ID NO: 40 or 41). In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO: 40 or 41), such as set forth in SEQ ID NO:68 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:42.

In some embodiments, the G protein or the functionally active variant or biologically active portion thereof binds to Ephrin B2 or Ephrin B3. In some aspects, the G protein has the sequence of amino acids set forth in any one of SEQ ID NO:41, SEQ ID NO:40 or SEQ ID NO:8, SEQ ID NO:10, SEQ ID NO: 9, SEQ ID NO:12 or SEQ ID NO:14, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:41, SEQ ID NO:40 or SEQ ID NO:8, SEQ ID NO:10, SEQ ID NO: 9, SEQ ID NO:12 or SEQ ID NO:14, or a functionally active variant or biologically active portion thereof, and retains binding to Ephrin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, and retains binding to Ephrin B2 or B3. Reference to retaining binding to Ephrin B2 or B3 includes binding that is at least or at least about 5% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 10% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 15% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 20% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 25% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion, 30% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 35% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 40% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 45% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 50% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 55% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 60% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 65% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, 70% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 75% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 80% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 85% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, such as at least or at least about 90% of the level or degree of binding of the corresponding wild-type G protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof, or such as at least or at least about 95% of the level or degree of binding of the corresponding wild-type protein, such as set forth in SEQ ID NO:44, SEQ ID NO:40 or SEQ ID NO:8, SEQ ID NO:10, SEQ ID NO: 9, SEQ ID NO:12 or SEQ ID NO:14, or a functionally active variant or biologically active portion thereof. In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:8, SEQ ID NO:9 or SEQ ID NO:44, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID SEQ ID NO:8, SEQ ID NO:9 or SEQ ID NO:44 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type NiV-G, such as set forth in SEQ ID NO:8, SEQ ID NO:9 or SEQ ID NO:44, 10% of the level or degree of binding of the corresponding wild-type NiV-G, such as set forth in SEQ ID NO:8, SEQ ID NO:9 or SEQ ID NO:44, 15% of the level or degree of binding of the corresponding wild-type NiV-G, such as set forth in SEQ ID NO:8, SEQ ID NO:9 or SEQ ID NO:44, 20% of the level or degree of binding of the corresponding wild-type NiV-G, such as set forth in SEQ ID NO:8, SEQ ID NO:9 or SEQ ID NO:44, 25% of the level or degree of binding of the corresponding wild-type NiV-G, such as set forth in SEQ ID NO:8, SEQ ID NO:9 or SEQ ID NO:4444, 30% of the level or degree of binding of the corresponding wild-type NiV-G, such as set forth in SEQ ID NO:8, SEQ ID NO:9 9 or SEQ ID NO:44, 35% of the level or degree of binding of the corresponding wild-type NiV-G, such as set forth in SEQ ID NO:8, SEQ ID NO:9 or SEQ ID NO:44, 40% of the level or degree of binding of the corresponding wild-type NiV-G, such as set forth in SEQ ID NO:8, SEQ ID NO:9 or SEQ ID NO:44, 45% of the level or degree of binding of the corresponding wild-type NiV-G, such as set forth in SEQ ID NO:8, SEQ ID NO:9 or SEQ ID NO:4450% of the level or degree of binding of the corresponding wild-type NiV-G, such as set forth in SEQ ID NO:8, SEQ ID NO:9 or SEQ ID NO:44, 55% of the level or degree of binding of the corresponding wild-type NiV-G, such as set forth in SEQ ID NO:8, SEQ ID NO:9 or SEQ ID NO:44, 60% of the level or degree of binding of the corresponding wild-type NiV-G, such as set forth in SEQ ID NO:8, SEQ ID NO:9 or SEQ ID NO:44, 65% of the level or degree of binding of the corresponding wild-type NiV-G, such as set forth in SEQ ID NO:8, SEQ ID NO:9 or SEQ ID NO:44, 70% of the level or degree of binding of the corresponding wild-type NiV-G, such as set forth in SEQ ID NO:8, SEQ ID NO:9 or SEQ ID NO:44, such as at least or at least about 75% of the level or degree of binding of the corresponding wild-type NiV-G, such as set forth in SEQ ID NO:8, SEQ ID NO:9 or SEQ ID NO:44, such as at least or at least about 80% of the level or degree of binding of the corresponding wild-type NIV-G, such as set forth in SEQ ID NO:8, SEQ ID NO:9 or SEQ ID NO:44, such as at least or at least about 85% of the level or degree of binding of the corresponding wild-type NiV-G, such as set forth in SEQ ID NO:8, SEQ ID NO:9 or SEQ ID NO:44, such as at least or at least about 90% of the level or degree of binding of the corresponding wild-type NiV-G, such as set forth in SEQ ID NO:8, SEQ ID NO:9 or SEQ ID NO:44, or such as at least or at least about 95% of the level or degree of binding of the corresponding wild-type NiV-G, such as set forth in SEQ ID NO:8, SEQ ID NO:9 or SEQ ID NO:44.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type NiV-G and exhibits reduced binding to one or both of the native binding partners Ephrin B2 or Ephrin B3. In some embodiments, the mutant G-protein or the biologically active portion, such as mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:40 or 41, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 40 or 41 and retains binding to Ephrin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO: 40 or 41, 10% of the level or degree of binding of the corresponding wild-type HeV-G, such as set forth in SEQ ID NO: 40 or 41, 15% of the level or degree of binding of the corresponding wild-type HeV-G, such as set forth in SEQ ID NO:66 or 67, 20% of the level or degree of binding of the corresponding wild-type HeV-G, such as set forth in SEQ ID NO: 40 or 41, 25% of the level or degree of binding of the corresponding wild-type HeV-G, such as set forth in SEQ ID NO: 40 or 41, 30% of the level or degree of binding of the corresponding wild-type HeV-G, such as set forth in SEQ ID NO: 40 or 41, 35% of the level or degree of binding of the corresponding wild-type HeV-G, such as set forth in SEQ ID NO: 40 or 41, 40% of the level or degree of binding of the corresponding wild-type HeV-G, such as set forth in SEQ ID NO: 40 or 41, 45% of the level or degree of binding of the corresponding wild-type HeV-G, such as set forth in SEQ ID NO: 40 or 41, 50% of the level or degree of binding of the corresponding wild-type HeV-G, such as set forth in SEQ ID NO: 40 or 41, 55% of the level or degree of binding of the corresponding wild-type HeV-G, such as set forth in SEQ ID NO: 40 or 41, 60% of the level or degree of binding of the corresponding wild-type HeV-G, such as set forth in SEQ ID NO: 40 or 41, 65% of the level or degree of binding of the corresponding wild-type HeV-G, such as set forth in SEQ ID NO: 40 or 41, 70% of the level or degree of binding of the corresponding wild-type HeV-G, such as set forth in SEQ ID NO:18 or 52, such as at least or at least about 75% of the level or degree of binding of the corresponding wild-type HeV-G, such as set forth in SEQ ID NO: 40 or 41, such as at least or at least about 80% of the level or degree of binding of the corresponding wild-type NIV-G, such as set forth in SEQ ID NO: 40 or 41, such as at least or at least about 85% of the level or degree of binding of the corresponding wild-type HeV-G, such as set forth in SEQ ID NO: 40 or 41, such as at least or at least about 90% of the level or degree of binding of the corresponding wild-type HeV-G, such as set forth in SEQ ID NO: 40 or 41, or such as at least or at least about 95% of the level or degree of binding of the corresponding wild-type HeV-G, such as set forth in SEQ ID NO:40 or 41.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type NiV-G and exhibits reduced binding to one or both of the native binding partners Ephrin B2 or Ephrin B3. In some embodiments, the mutant G-protein or the biologically active portion, such as mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:38.

In some embodiments, the G protein is a mutant G protein containing one or more amino acid substitutions selected from the group consisting of E501A, W504A, Q530A and E533A with reference to numbering set forth in SEQ ID NO:8. In some embodiments, the G protein is a mutant G protein that contains one or more amino acid substitutions elected from the group consisting of E501A, W504A, Q530A and E533A with reference to SEQ ID NO:8and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO: 8), 6 contiguous amino acid residues at or near the N-terminus of the wild-type NiV-G protein (SEQ ID NO: 8), 7 contiguous amino acid residues at or near the N-terminus of the wild-type NiV-G protein (SEQ ID NO: 8), 8 contiguous amino acid residues at or near the N-terminus of the wild-type NiV-G protein (SEQ ID NO: 8), 9 contiguous amino acid residues at or near the N-terminus of the wild-type NiV-G protein (SEQ ID NO: 8), up to 10 contiguous amino acid residues at or near the N-terminus of the wild-type NiV-G protein (SEQ ID NO: 8), 11 contiguous amino acid residues at or near the N-terminus of the wild-type NiV-G protein (SEQ ID NO: 8), 12 contiguous amino acid residues at or near the N-terminus of the wild-type NiV-G protein (SEQ ID NO: 8), 13 contiguous amino acid residues at or near the N-terminus of the wild-type NiV-G protein (SEQ ID NO: 8), 14 contiguous amino acid residues at or near the N-terminus of the wild-type NiV-G protein (SEQ ID NO: 8), up to 15 contiguous amino acid residues at or near the N-terminus of the wild-type NiV-G protein (SEQ ID NO: 8), 16 contiguous amino acid residues at or near the N-terminus of the wild-type NiV-G protein (SEQ ID NO: 8), 17 contiguous amino acid residues at or near the N-terminus of the wild-type NiV-G protein (SEQ ID NO: 8), 18 contiguous amino acid residues at or near the N-terminus of the wild-type NiV-G protein (SEQ ID NO: 8), 19 contiguous amino acid residues at or near the N-terminus of the wild-type NiV-G protein (SEQ ID NO: 8), up to 20 contiguous amino acid residues at or near the N-terminus of the wild-type NiV-G protein (SEQ ID NO: 8), 21 contiguous amino acid residues at or near the N-terminus of the wild-type NiV-G protein (SEQ ID NO: 8) 22 contiguous amino acid residues at or near the N-terminus of the wild-type NiV-G protein (SEQ ID NO: 8), 23 contiguous amino acid residues at or near the N-terminus of the wild-type NiV-G protein (SEQ ID NO: 8), 24 contiguous amino acid residues at or near the N-terminus of the wild-type NiV-G protein (SEQ ID NO: 8), up to 25 contiguous amino acid residues at or near the N-terminus of the wild-type NiV-G protein (SEQ ID NO: 8), 26 contiguous amino acid residues at or near the N-terminus of the wild-type NiV-G protein (SEQ ID NO: 8), 27 contiguous amino acid residues at or near the N-terminus of the wild-type NiV-G protein (SEQ ID NO: 8), 28 contiguous amino acid residues at or near the N-terminus of the wild-type NiV-G protein (SEQ ID NO: 8), 29 contiguous amino acid residues at or near the N-terminus of the wild-type NiV-G protein (SEQ ID NO: 8), up to 30 contiguous amino acid residues at or near the N-terminus of the wild-type NiV-G protein (EQ ID NO: 8), up to 31 contiguous amino acid residues at or near the N-terminus of the wild-type NiV-G protein (SEQ ID NO: 8), 32 contiguous amino acid residues at or near the N-terminus of the wild-type NiV-G protein (SEQ ID NO: 8), 33 contiguous amino acid residues at or near the N-terminus of the wild-type NiV-G protein (SEQ ID NO: 8) 34 contiguous amino acid residues at or near the N-terminus of the wild-type NiV-G protein (SEQ ID NO: 8), 35 contiguous amino acid residues at or near the N-terminus of the wild-type NiV-G protein (SEQ ID NO: 8) up to 36 contiguous amino acid residues at or near the N-terminus of the wild-type NiV-G protein (EQ ID NO: 8), up to 37 contiguous amino acid residues at or near the N-terminus of the wild-type NiV-G protein (EQ ID NO: 8), up to 38 contiguous amino acid residues at or near the N-terminus of the wild-type NiV-G protein (EQ ID NO: 8), up to 39 contiguous amino acid residues at or near the N-terminus of the wild-type NiV-G protein (SEQ ID NO: 8), or up to 40 contiguous amino acid residues at or near the N-terminus of the wild-type NiV-G protein (EQ ID NO: 8).

In some embodiments, the mutant NiV-G protein has the amino acid sequence set forth in SEQ ID NO: 34 or 35 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 34 or 35. In particular embodiments, the G protein has the sequence of amino acids set forth in SEQ ID NO 34 or 35.

In some embodiments, the G protein is a mutant G protein containing one or more amino acid substitutions selected from the group consisting of E501A, W504A, Q530A and E533A with reference to numbering set forth in SEQ ID NO:10. In some embodiments, the G protein is a mutant G protein that contains one or more amino acid substitutions elected from the group consisting of E501A, W504A, Q530A and E533A with reference to SEQ ID NO:10 and is a biologically active portion thereof containing an N-terminal truncation.

b. F Proteins

In some embodiments, the vector-surface targeting moiety comprises a protein with a hydrophobic fusion peptide domain. In some embodiments, the vector-surface targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

Table 4B provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:16). Following cleavage of the signal peptide, the mature F₀ (e.g. SEQ ID NO:17) is transported to the cell surface, then endocytosed and cleaved by cathepsin L into the mature fusogenic subunits F1 and F2. The F1 and F2 subunits are associated by a disulfide bond and recycled back to the cell surface. The F1 subunit contains the fusion peptide domain located at the N terminus of the F1 subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 4B
F proteins

| | | | SEQ ID |
|---|---|---|---|
| Full | | | (without |
| Gene | | SEQ | signal |
| Name | Sequence | ID | sequence) |
| Hendra | MATQEVRLKCLLCGIIVLVLSLEGLGILHY | 16 | 17 |
| virus | EKLSKIGLVKGITRKYKIKSNPLTKDIVIK | | |
| F | MIPNVSNVSKCTGTVMENYKSRLTGILSPI | | |
| Protein | KGAIELYNNNTHDLVGDVKLAGVVMAGIAI | | |
| | GIATAAQITAGVALYEAMKNADNINKLKSS | | |
| | IESTNEAVVKLQETAEKTVYVLTALQDYIN | | |
| | TNLVPTIDQISCKQTELALDLALSKYLSDL | | |
| | LFVFGPNLQDPVSNSMTIQAISQAFGGNYE | | |
| | TLLRTLGYATEDFDDLLESDSIAGQIVYVD | | |
| | LSSYYIIVRVYFPILTEIQQAYVQELLPVS | | |
| | FNNDNSEWISIVPNFVLIRNTLISNIEVKY | | |
| | CLITKKSVICNQDYATPMTASVRECLTGST | | |
| | DKCPRELVVSSHVPRFALSGGVLFANCISV | | |
| | TCQCQTTGRAISQSGEQTLLMIDNTTCTTV | | |
| | VLGNIIISLGKYLGSINYNSESIAVGPPVY | | |
| | TDKVDISSQISSMNQSLQQSKDYIKEAQKI | | |
| | LDTVNPSLISMLSMIILYVLSIAALCIGLI | | |
| | TFISFVIVEKKRGNYSRLDDRQVRPVSNGD | | |
| | LYYIGT | | |
| Nipah | MVVILDKRCYCNLLILILMISECSVGILHY | 18 | 19 |
| virus | EKLSKIGLVKGVTRKYKIKSNPLTKDIVIK | | |
| F | MIPNVSNMSQCTGSVMENYKTRLNGILTPI | | |
| Protein | KGALEIYKNNTHDLVGDVRLAGVIMAGVAI | | |
| | GIATAAQITAGVALYEAMKNADNINKLKSS | | |
| | IESTNEAVVKLQETAEKTVYVLTALQDYIN | | |
| | TNLVPTIDKISCKQTELSLDLALSKYLSDL | | |
| | LFVFGPNLQDPVSNSMTIQAISQAFGGNYE | | |
| | TLLRTLGYATEDFDDLLESDSITGQIIYVD | | |
| | LSSYYIIVRVYFPILTEIQQAYIQELLPVS | | |
| | FNNDNSEWISIVPNFILVRNTLISNIEIGF | | |
| | CLITKRSVICNQDYATPMTNNMRECLTGST | | |
| | EKCPRELVVSSHVPRFALSNGVLFANCISV | | |
| | TCQCQTTGRAISQSGEQTLLMIDNTTCPTA | | |
| | VLGNVIISLGKYLGSVNYNSEGIAIGPPVF | | |
| | TDKVDISSQISSMNQSLQQSKDYIKEAQRL | | |
| | LDTVNPSLISMLSMIILYVLSIASLCIGLI | | |
| | TFISFIIVEKKRNTYSRLEDRRVRPTSSGD | | |
| | LYYIGT | | |
| Cedar | MSNKRTTVLIIISYTLFYLNNAAIVGFDFD | 20 | 21 |
| Virus | KLNKIGVVQGRVLNYKIKGDPMTKDLVLKF | | |
| F | IPNIVNITECVREPLSRYNETVRRLLLPIH | | |
| Protein | NMLGLYLNNTNAKMTGLMIAGVIMGGIAIG | | |
| | IATAAQITAGFALYEAKKNTENIQKLTDSI | | |
| | MKTQDSIDKLTDSVGTSILILNKLQTYINN | | |
| | QLVPNLELLSCRQNKIEFDLMLTKYLVDLM | | |
| | TVIGPNINNPVNKDMTIQSLSLLFDGNYDI | | |
| | MMSELGYTPQDFLDLIESKSITGQIIYVDM | | |
| | ENLYVVIRTYLPTLIEVPDAQIYEFNKITM | | |
| | SSNGGEYLSTIPNFILIRGNYMSNIDVATC | | |
| | YMTKASVICNQDYSLPMSQNLRSCYQGETE | | |
| | YCPVEAVIASHSPRFALTNGVIFANCINTI | | |
| | CRCQDNGKTITQNINQFVSMIDNSTCNDVM | | |
| | VDKFTIKVGKYMGRKDINNINIQIGPQIII | | |
| | DKVDLSNEINKMNQSLKDSIFYLREAKRIL | | |
| | DSVNISLISPSVQLFLIIISVLSFIILLII | | |
| | IVYLYCKSKHSYKYNKFIDDPDYYNDYKRE | | |
| | RINGKASKSNNIYYVGD | | |
| Mojiang | MALNKNMFSSLFLGYLLVYATTVQSSIHYD | 22 | 23 |
| virus, | SLSKVGVIKGLTYNYKIKGSPSTKLMVVKL | | |
| Tongguan | IPNIDSVKNCTQKQYDEYKNLVRKALEPVK | | |
| 1 F | MAIDTMLNNVKSGNNKYRFAGAIMAGVALG | | |
| Protein | VATAATVTAGIALHRSNENAQAIANMKSAI | | |
| | QNTNEAVKQLQLANKQTLAVIDTIRGEINN | | |
| | NIIPVINQLSCDTIGLSVGIRLTQYYSEII | | |
| | TAFGPALQNPVNTRITIQAISSVFNGNFDE | | |
| | LLKIMGYTSGDLYEILHSELIRGNIIDVDV | | |
| | DAGYIALEIEFPNLTLVPNAVVQELMPISY | | |
| | NIDGDEWVTLVPRFVLTRTTLLSNIDTSRC | | |
| | TITDSSVICDNDYALPMSHELIGCLQGDTS | | |
| | KCAREKVVSSYVPKFALSDGLVYANCLNTI | | |
| | CRCMDTDTPISQSLGATVSLLDNKRCSVYQ | | |
| | VGDVLISVGSYLGDGEYNADNVELGPPIVI | | |
| | DKIDIGNQLAGINQTLQEAEDYIEKSEEFL | | |
| | KGVNPSIITLGSMVVLYIFMILIAIVSVIA | | |
| | LVLSIKLTVKGNVVRQQFTYTQHVPSMENI | | |
| | NYVSH | | |
| Bat | MKKKTDNPTISKRGHNHSRGIKSRALLRET | 24 | 25 |
| Paramy- | DNYSNGLIVENLVRNCHHPSKNNLNYTKTQ | | |
| xovirus | KRDSTIPYRVEERKGHYPKIKHLIDKSYKH | | |
| Eid_hel/ | IKRGKRRNGHNGNIITIILLLILILKTQMS | | |
| GH-M74a/ | EGAIHYETLSKIGLIKGITREYKVKGTPSS | | |
| GHA/2009 | KDIVIKLIPNVTGLNKCTNISMENYKEQLD | | |
| F | KILIPINNIIELYANSTKSAPGNARFAGVI | | |
| protein | IAGVALGVAAAAQITAGIALHEARQNAERI | | |
| | NLLKDSISATNNAVAELQEATGGIVNVITG | | |
| | MQDYINTNLVPQIDKLQCSQIKTALDISLS | | |
| | QYYSEILTVFGPNLQNPVTTSMSIQAISQS | | |
| | FGGNIDLLLNLLGYTANDLLDLLESKSITG | | |
| | QITYINLEHYFMVIRVYYPIMTTISNAYVQ | | |
| | ELIKISFNVDGSEWVSLVPSYILIRNSYLS | | |
| | NIDISECLITKNSVICRHDFAMPMSYTLKE | | |
| | CLTGDTEKCPREAVVTSYVPRFAISGGVIY | | |
| | ANCLSTTCQCYQTGKVIAQDGSQTLMMIDN | | |
| | QTCSIVRIEEILISTGKYLGSQEYNTMHVS | | |
| | VGNPVFTDKLDITSQISNINQSIEQSKFYL | | |
| | DKSKAILDKINLNLIGSVPISILFIIAILS | | |
| | LILSIITFVIVMIIVRRYNKYTPLINSDPS | | |
| | SRRSTIQDVYIIPNPGEHSIRSAARSIDRD | | |
| | RD | | |

In some embodiments, the F protein is encoded by a nucleotide sequence that encodes the sequence set forth by any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25.

In particular embodiments, the F protein or the functionally active variant or biologically active portion thereof retains fusogenic activity in conjunction with a Henipavirus G protein, such as a G protein set forth in Section II.E.1a (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L(e.g. corresponding to the cleavage site between amino acids 109-110 of SEQ ID NO:18).

In particular embodiments, the F protein has the sequence of amino acids set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25., or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25.

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, or SEQ ID NO:25,

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 16-25. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F₀ precursor is encoded by a sequence of nucleotides encoding the sequence set forth in SEQ ID NO: 36. The encoding nucleic acid can encode a signal peptide sequence that has the sequence MVVILDKRCY CNLLILILMI SECSVG (SEQ ID NO: 26). In some embodiments, the F protein has the sequence set forth in SEQ ID NO:18. In some examples, the F protein is cleaved into an F1 subunit comprising the sequence set forth in SEQ ID NO:28 and an F2 subunit comprising the sequence set forth in SEQ ID NO: 27.

In some embodiments, the F protein is a NiV-F protein that is encoded by a sequence of nucleotides encoding the sequence set forth in SEQ ID NO:18, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 18. In some embodiments, the NiV-F-protein has the sequence of set forth in SEQ ID NO: 11, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In particular embodiments, the F protein or the functionally active variant or biologically active portion thereof retains the cleavage site cleaved by cathepsin L.

In some embodiments, the F protein or the functionally active variant or the biologically active portion thereof includes an F1 subunit that has the sequence set forth in SEQ ID NO: 28, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:28.

In some embodiments, the F protein or the functionally active variant or biologically active portion thereof includes an F2 subunit that has the sequence set forth in SEQ ID NO: 27, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:27.

In some embodiments, the F protein or the functionally active variant or the biologically active portion thereof includes an F1 subunit that has the sequence set forth in SEQ ID NO: 9, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:9.

In some embodiments, the F protein or the functionally active variant or biologically active portion thereof includes an F2 subunit that has the sequence set forth in SEQ ID NO: 10, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:10.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:28). In some embodiments, the mutant NiV-F protein comprises an amino acid sequence set forth in SEQ ID NO:11. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In some embodiments, the mutant F protein contains an F1 protein that has the sequence set forth in SEQ ID NO:12. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12.

In some embodiments, the F protein is a mutant NiV-F protein that is a biologically active portion thereof that comprises a 20 amino acid truncation at or near the C-terminus of the wild-type NiV-F protein (SEQ ID NO:28); and a point mutation on an N-linked glycosylation site. In some embodiments, the mutant NiV-F protein comprises an amino acid sequence set forth in SEQ ID NO: 13. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 13.

In some embodiments, the F protein is a mutant NiV-F protein that is a biologically active portion thereof that comprises a 22 amino acid truncation at or near the C-terminus of the wild-type NiV-F protein (SEQ ID NO:28). In some embodiments, the NiV-F protein is encoded by a nucleotide sequence that encodes the sequence set forth in SEQ ID NO: 14. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 14.

2 Retargeting Moieties

In some embodiments, the fusogen is a targeted envelope protein that contains a vector-surface targeting moiety. In some embodiments, the vector-surface targeting moiety binds a target ligand. In some embodiments, the target ligand can be expressed in an organ or cell type of interest, e.g., the lung. In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred.

In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)₂, Fv fragments, scFv antibody fragments, disulfide-linked Fvs (sdFv), a Fd fragment consisting of the V_{H} and CH1 domains, linear antibodies, single domain antibodies such as sdAb (either VL or V_{H}), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nm1192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

In some embodiments, vector-surface targeting moiety is a peptide. In some embodiments, vector-surface targeting moiety is an antibody, such as a single domain antibody. In some embodiments, the antibody can be human or humanized. In some embodiments, antibody or portion thereof is naturally occurring. In some embodiments, the antibody or portion thereof is synthetic.

In some embodiments, the antibody can be generated from phage display libraries to have specificity for a desired target ligand. In some embodiments the target ligand is expressed in the lung, such as ACE2. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the vector-surface targeting moiety is attached to the C-terminus of the G protein (e.g., fusogen) or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the vector-surface targeting moiety specifically binds to a cell surface molecule present on a target cell. In some embodiments, the vector-surface targeting moiety is a protein, glycan, lipid or low molecular weight molecule.

In some embodiments, the vector-surface targeting moiety is derived from a coronavirus. Coronaviruses typically bind to target cells through Spike-receptor (S) interactions and enter cells by receptor mediated endocytosis or fusion with the plasma membrane. The S-receptor interaction is a strong determinant of species specificity as demonstrated for both group 1 and group 2 coronaviruses. The receptor for group 1 coronaviruses, including human coronavirus 229E (HCoV-229E), feline coronavirus (FCoV) and porcine coronavirus (PCoV) has been identified as aminopeptidase N (APN/CD13) (Delmas, et al., 1992, Nature 357:417-420; Tresnan, et al., 1996, J. Virol. 70:8669-8674; Yeager, et al., 1992, Nature 357:420-422). APN/CD13 is a 150- to 160-kDa type II protein that is a membrane peptidase (Look, et al., 1989, J. Clin. Invest 83:1299-1307). In some embodiments, the S protein binds ACE2. In some embodiments, the S protein binds DPP4.

In some embodiments, the vector-surface targeting moiety is derived from a coronavirus S protein. The coronavirus S glycoprotein is exemplified, but not limited to, those encoded by the genomic sequences in gi|31416292|gb|AY278487.3| SARS coronavirus BJ02,gi|30248028|gb|AY274119.3: SARS coronavirus TOR2, gi|30698326|gb|AY291451.1: SARS coronavirus TW1, gi|33115118|gb|AY323977.2 SARS coronavirus HSR 1, gi|35396382|gb|AY394850.1: SARS coronavirus WHU, gi|33411459|dbj|AP006561.1: SARS coronavirus TWY, gi|33411444|dbj|AP006560.1: SARS coronavirus TWS, gi|33411429|dbj|AP006559.1 SARS coronavirus TWK, gi|33411414|dbj|AP006558.1 SARS coronavirus TWJ, gi|33411399|dbj|AP006557.1| SARS coronavirus TWH, gi|30023963|gb|AY278491.2: SARS coronavirus HKU-39849, gi|33578015|gb|AY310120.1: SARS coronavirus FRA, gi|33518725|gb|AY362699.1| SARS coronavirus TWC3, gi|33518724|gb|AY362698| SARS coronavirus TWC2, gi|30027617|gb|AY278741.1: SARS coronavirus Urbani, gi|31873092|gb|AY321118.1: SARS coronavirus TWC, gi|33304219|gb|AY351680.1: SARS coronavirus ZMY 1, gi|31416305|gb|AY278490.3° SARS coronavirus BJ03, gi|30910859|gb|AY297028.1: SARS coronavirus ZJO1, gi|30421451|gb|AY282752.1: SARS coronavirus CUHK-Sul0, SARS coronavirus SZ16, gi|34482137|gb|AY304486.1: SARS coronavirus SZ3 gi|30027610|gb|AY278554.2 SARS coronavirus CUHK-W1, gi|31416306|gb|AY279354.2: SARS coronavirus BJO4, gi|37576845|gb|AY427439.1|SARS coronavirus AS, gi|37361915|gb|AY283798.2| SARS coronavirus Sin2774, gi|31416290|gb|AY278489.2| SARS coronavirus GDO1, gi|30468042|gb|AY283794.1: SARS coronavirus Sin2500, gi|30468043|gb|AY283795.1: SARS coronavirus Sin2677, gi|30468044|gb|AY283796.1: SARS coronavirus Sin2679, gi|30468045|gb|AY283797.1| SARS coronavirus Sin2748, gi|31982987|gb|AY286320.2| SARS coronavirus isolate ZJ-HZ01, and gi|30275666|gb|AY278488.2| SARS coronavirus BJO1.

In some embodiments, the vector-surface targeting moiety is Severe Acute Respiratory Syndrome (SARS) coronavirus 1 (SARS CoV-1) spike glycoprotein. In some embodiments, the vector-surface targeting moiety is Severe Acute Respiratory Syndrome (SARS) coronavirus 2 (SARS CoV-2) spike glycoprotein. In some embodiments, the vector-surface targeting moiety is syncytin,

In some embodiments, the cell surface ligand of a target cell is an antigen or portion thereof. In some embodiments, the vector-surface targeting moiety or portion thereof is an antibody having a single monomeric domain antigen binding/recognition domain that is able to bind selectively to a specific antigen. In some embodiments, the single domain antibody binds an antigen present on a target cell.

Exemplary cells include lung stem cells, bronchiolar epithelial cells, alveolar epithelial cells, stromal cells, type 1 and II pneumocytes also known as alveolar type I and II epithelial cells, basal cells, secretory cells, club cells, clara cells, ciliated cells, capillary cells, alveolar macrophages, and lung epithelial cells. In some embodiments, the target cell is an epithelial cell. In some embodiments, the ligand is expressed on a host cell, such as an epithelial cell. In some embodiments, the ligand is ACE2. In some embodiments, the target cell is B-lymphocyte. In some embodiments, the ligand is CD20.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments, the target tissue is the lung.

### III. Pharmaceutical Composition and Methods of Manufacture

Also provided are compositions containing the binding agent, particles, or polynucleotides encoding the binding agents, including pharmaceutical compositions and formulations. Also provided are compositions containing any of the provided vehicles, such as virus-like particles, containing the binding agent or polynucleotides encoding the binding agents, or provided particles including pharmaceutical compositions and formulations. Also provided are methods of using and uses of the compositions, such as in the treatment of coronavirus infection.

The present disclosure also provides, in some aspects, a pharmaceutical composition comprising the composition described herein and pharmaceutically acceptable carrier. The pharmaceutical compositions can include any of the described polynucleotides or vehicles for delivery.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

A pharmaceutical composition may be prepared, packaged, and/or sold in a formulation suitable for pulmonary administration via the buccal cavity, such as via inhalation. Delivery systems currently available include pressurized metered dose inhalator, nebulisers, and dry powder inhalers. In some aspects, it has been found that medicaments for administration by inhalation should be of a controlled particle size in order to achieve maximum penetration into the lungs, preferably in the range of 1 to 10 micrometers in diameter.

Pharmaceutical compositions formulated for pulmonary delivery may provide an active ingredient in the form of droplets of a solution and/or suspension. Such compositions may be prepared, packaged, and/or sold as aqueous and/or dilute alcoholic solutions and/or suspensions, such as sterile, comprising active ingredient, and may conveniently be administered using any nebulization and/or atomization device. Such compositions may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

In some aspects, the polynucleotides, polypeptides, and vehicles provided herein can be formulated in compositions suitable for inhalation, including, for example, inhalable powders, propellant-containing aerosols and propellant-free inhalation solutions. In certain embodiments, the inhalable powder is administered to the subject via a dry powder inhaler (DPI). In certain embodiments, a propellant-containing aerosol is administered to a subject via a metered dose inhaler (MDI). In certain embodiments, the propellant-free inhalation solution is administered to the subject via a nebulizer.

In some embodiments, the composition containing any of the provided agent or vehicle, e.g. polynucleotides, binding agents or particles, or vehicles for delivery, are freeze dried, such as in the form of a dry powder. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50%) to 99.9%) (w/w) of the composition, and active ingredient may constitute 0.1%> to 20% (w/w) of the composition. A propellant may further comprise additional ingredients such as a liquid non-ionic and/or solid anionic surfactant and/or a solid diluent (which may have a particle size of the same order as particles comprising the active ingredient).

In some aspects, a pharmaceutical composition comprising the polynucleotides, poplypeptides, and/or vehicles provided herein can be prepared for inhalation with an amount of at least one surfactant, such as is sufficient to facilitate the absorption of inhaled particles. To obtain these absorbable compositions, any surfactant that facilitates inhalation of any of the compositions disclosed herein. Surfactants suitable for use in promoting absorption of inhaled compositions include, but are not limited to, polyoxyethylene sorbitol esters such as polysorbate 80 (Tween 80) and polysorbate 20 (Tween 20); Propylene-polyoxyethylene esters such as poloxamer 188; polyoxyethylene alcohols such as Brij35; mixtures of polysorbate surfactants with phospholipids such as phosphatidylcholine and derivatives (dipalmitoyl, dioleoyl, dimyristyl, or 1-palmitoyl, 2 Mixed derivatives such as olcoyl), dimyristol glycerol and other members of a series of phospholipid glycerols; lysophosphatidylcholine and derivatives thereof; lysolecithin A mixture of polysorbate with cholesterol; a mixture of polysorbate surfactant with sorbitan surfactant (such as sorbitan monooleate, dioleate, trioleate or others from this class); poloxamer surfactants; bile salts and their Derivatives such as sodium cholate, sodium deoxycholate, sodium glycodeoxycholate, sodium taurocholate, etc.; mixed micelles of TNFα inhibitors with bile salts and phospholipids; Brij surfactant (such as Brij35-PEG923) lauryl alcohol, etc.) Is included. The amount of surfactant to be added is about 0.005% to about 1.0% (w/v), preferably about 0.005% to about 0.5%, more preferably about 0.01%. To about 0.4%, even more preferably from about 0.03% to about 0.3%, and most preferably from about 0.05% to about 0.2%.

Sterile inhalation solutions can be prepared by incorporating the required amount of the active compound (i.e., polynucleotides, polypeptides, and/or vehicles comprising a binding agent or particle) in an appropriate solvent. Solutions can then be sterilized by filtration. Generally, dispersions are prepared by incorporating the active compound into a sterile vehicle that contains a basic dispersion medium and the required other ingredients from those enumerated above. The correct fluidity of the solution can be maintained, for example, by the use of a coating such as lecithin, by the maintenance of the required particle size in the case of dispersion and by the use of surfactants. Inclusion of agents that delay absorption, such as monostearate salts and gelatin, in the composition may provide sustained absorption of the inhalable composition.

Compositions described herein as being useful for pulmonary delivery are useful for intranasal delivery of a pharmaceutical composition. Another compositions suitable for intranasal administration is a coarse powder comprising the active ingredient and having an average particle from about 0.2 μlη to 500 μlη. Such a compositions is administered in the manner in which snuff is taken, i.e. by rapid inhalation through the nasal passage from a container of the powder held close to the nose. Compositions suitable for nasal administration may, for example, comprise from about as little as 0.1% (w/w) and as much as 100%) (w/w) of active ingredient, and may comprise one or more of the additional ingredients described herein.

A pharmaceutical composition may be prepared, packaged, and/or sold in a formulation suitable for buccal administration. Such compositions may, for example, be in the form of tablets and/or lozenges made using conventional methods, and may, for example, 0.1% to 20% (w/w) active ingredient, the balance comprising an orally dissolvable and/or degradable composition and, one or more of the additional ingredients described herein. Alternately, compositions suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Active ingredients may be entrapped in microcapsules, in colloidal drug delivery systems (for example, liposomes, albumin microspheres, microemulsions, nanoparticles and nanocapsules) or in macroemulsions. In certain embodiments, the pharmaceutical composition is formulated as an inclusion complex, such as cyclodextrin inclusion complex, or as a liposome. Liposomes can serve to target the polynucleotides (e.g., delivery vehicles) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some embodiments contains polynucleotides or vehicles for their delivery in amounts effective to treat a coronavirus infection, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The polynucleotides and/or delivery vehicle may be administered using standard administration techniques, formulations, and/or devices. Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the polynucleotides and/or delivery vehicle are administered by nebulization. In some embodiments, the polynucleotides and/or delivery vehicle are administered by inhalation.

In some embodiments, the vehicle for delivery of a polynucleotide or polypeptide, such as encoding a binding agent or particle, is a viral vector or virus-like particle (E.g., Section II). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the vehicle for delivery of a polynucleotide or polypeptide, such as encoding a binding agent or particle, is an adenovirus vector. In some aspects, the dosage for administration of adenovirus to humans can range from about 10⁷ to 10⁹, inclusive, plaque forming units (pfu) per injection.

In some aspects, the dosage of administration of a vehicle within the pharmaceutical compositions provided herein varies depending on a subject's body weight. For example, a composition may be formulated as GC/kg, infectious units/kg, pfu/kg, etc. In some aspects, the dosage at which a therapeutic effect is obtained is from at or about 10⁸ GC/kg to at or about 10¹⁴ GC/kg of the subject's body weight, inclusive. In some aspects, the dosage at which a therapeutic effect is obtained is at or about 10⁸ GC/kg of the subject's body weight (GC/kg).

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein in Section V and/or by detecting a desired response.

The exact amount of the polynucleotide, polypeptide, or vector vehicle required will vary from subject to subject, depending on the species, age, weight and general condition of the subject, the particular poylnucleic acid, polypeptide, or vector used, its mode of administration etc. Thus, it is not possible to specify an exact amount for every polynucleic acid, polynucleotide, or vector vehicle provided herein. However, an appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cyroprotectants are combined with vector vehicles in order to stabilize them during freezing. In some aspects, Frozen storage of RNA between −20° C. and −80° C. may be advantageous for long term (e.g. 36 months) stability of polynucleotide. In some embodiments, the RNA species is mRNA. In some embodiments, cyroprotectants are included in vehicle formulations to stabilize polynucleotide through freeze/thaw cycles and under frozen storage conditions. Cyroprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### IV. Methods of Use and Therapeutic Applications

Provided herein are methods and uses of treating a virus infection, such as a coronavirus infection. In some embodiments polypeptide binding agents, or polynucleotides encoding the binding agent, or the provided particles can be administered to subjects for treating or reducing a viral infection in a subject, according to the provided embodiments and methods. Among provided methods and uses are those involving administering compositions containing the binding agents or vehicles containing the binding agents to treat a virus infection, such as a coronavirus infection. Among provided methods and uses are those involving administering compositions containing the particles, or vehicles containing the particles provided herein to treat a virus infection, such as a coronavirus infection. Among provided methods and uses are those involving administering compositions containing polynucleotides encoding the binding agents, or vehicles containing the polypeptides, or vehicles containing the polynucleotides, to treat a virus infection, such as a coronavirus infection. In some embodiments, the disease or condition is a virus infection, and the subject is known, suspected, or predicted to have been exposed to a virus causing the infection.

In some embodiments, the virus is of the order Nidovirales. In some embodiments, the virus is of sub-order Cornidovirineae. In some embodiments, the virus is of family Coronaviridae. In some embodiments, the virus is of sub-family Orthocoronavirinae. In some aspects, the virus is of genera Alphacoronavirus. In some aspects, the virus is of genera Betacoronavirus.

In provided embodiments, the methods relate to treating a coronavirus infection. Coronaviridae is a family of related enveloped viruses. Coronaviruses feature positive sense single stranded RNA genomes within a helical nucelocapsid and icosahedral protein coat. The genome of a coronavirus can vary between 26 to roughly 32 kilobases, some of the largest viral genomes recorded. The first human coronavirus were discovered in the 1960's, including strains B814, 229E, IBV and OC43. More recently discovered human coronaviruses include NL63 and HKU1, identified in 2003 and 2004 respectively. Many human coronaviruses circulate in the population and cause seasonal epidemics and/or sporadic disease associated with the common cold.

In some embodiments, the virus is a coronavirus. In some embodiments, the coronavirus is of any of coronavirus subgroups: 1a, 1b, 2a, 2b, 2c, 2d, or 3.

In some embodiments, the virus is a subgroup 1a coronavirus. Nonlimiting examples of a subgroup 1a coronavirus of any of the provided embodiments include FCov.FIPV.79.1 146.VR.2202 (GenBank Accession No. NV_007025), transmissible gastroenteritis vims (TGEV) (GenBank Accession No. NC J302306; GenBank Accession No. Q81 1789.2; GenBank Accession No. DQ81 1786.2; GenBank Accession No. DQ811788.1; GenBank Accession No. DQ811785.1; GenBank Accession No. X52157.1; GenBank Accession No. AJ01 1482.1; GenBank Accession No. KC962433.1; GenBank Accession No. AJ271965.2; GenBank Accession No. JQ693060.1; GenBank Accession No. C609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_0019 1.1; GenBank Accession No. DQ81 1787), as well as any other subgroup 1a coronavirus now known (e.g., as can be found in the GenBank® Database) or later identified, and any combination thereof.

In some embodiments, the virus is a subgroup 1b coronavirus. Nonlimiting examples of a subgroup 1b coronavirus of any of the provided embodiments include BtCoV. 1A.AFCD62 (GenBank Accession No. NC_010437), BtCoV. 1B.AFCD307 (GenBank Accession No. NCJH0436), BtCov.H U8.AFCD77 (GenBank Accession No. NC_010438), BtCoV.512.2005 (GenBank Accession No. DQ648858), porcine epidemic diarrhea virus PEDV.CV777 (GenBank Accession No. NCJ)034365 GenBank Accession No. DQ355224.1, GenBank Accession No. DQ355223.1, GenBank Accession No. DQ355221.1, GenBank Accession No. JN601062.1, GenBank Accession No. JN601061.1, GenBank Accession No. JN601060.1, GenBank Accession No. J 601059.1, GenBank Accession No. JN601058.1, GenBank Accession NO.JN601057.1, GenBank Accession No, JN601056.1, GenBank Accession NO.JN60I 055, 1, GenBank Accession No. JN601054.1, GenBank Accession No. JN601053.1, GenBank Accession No. JN601052.1, GenBank Accession No. JN400902.1, GenBank Accession No.JN547395.1, GenBank Accession No. FJ687473.1, GenBank Accession No.FJ687472.1, GenBank Accession No. FJ687471.1, GenBank Accession No. FJ687470.1, GenBank Accession No. FJ687469.1, GenBank Accession No.FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No.FJ687462, 1, GenBank Accession No. FJ68746U, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No. FJ687449.1, GenBank Accession No. AF500215.1, GenBank Accession No. KF476061.1, GenBank Accession No. KF476060.1, GenBank Accession No. F476059.1, GenBank Accession No. KF476058.1, GenBank Accession No. KF476057.1, GenBank Accession No. F476056.1, GenBank Accession No. KF476055.1, GenBank Accession No, KF476054.1, GenBank Accession No. KF476053.1, GenBank Accession No. KF476052.1, GenBank Accession No. KF476051.1, GenBank Accession No. KF476050.1, GenBank Accession No. F476049.1, GenBank Accession No. KF476048.1, GenBank Accession No. KF 177258.1, GenBank Accession No. KF177257.1, GenBank Accession No. KF177256.1, GenBank Accession No. KF177255.1), HCoV.229E (GenBank Accession No. NCJ)02645), HCoV.NL63. Amsterdam.! (GenBank Accession No. NC_005831), BtCoV.H U2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

In some embodiments, the virus is a subgroup 2a coronavirus. Nonlimiting examples of a subgroup 2a coronavirus of any of the provided embodiments include HCoV.HKUl.CN5 (GenBank Accession No. DQ339101), MHV.A59 (GenBank Accession No. NC_001846), PHEV.VW572 (GenBank Accession No. NC_007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

In some embodiments, the virus is a subgroup 2b coronavirus. Nonlimiting examples of a subgroup 2b coronavirus of any of the provided embodiments include BtSARS.HKlD.1 (GenBank Accession No, DQ022305), BtSARS.HKU3.2 (GenBank Accession No. DQ084199), BtSARS.HKU3.3 (GenBank Accession No. DQ084200), BtSARS.Rml (GenBank Accession No. DQ412043), BtCoV.279.2005 (GenBank Accession No. DQ648857), BtSARS.Rfl (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GDOl (GenBank Accession No. AY278489), SARSCoV.HC.SZ.61.03 (GenBank Accession No. AY515512), SARSCoV.SZ 16 (GenBank Accession No. AY304488), SARSCoV.Urbani (GenBank Accession No. AY278741), SARSCoV.civetOlO (GenBank Accession No. AY572035), SARSCoV.MA.15 (GenBank Accession No. DQ497008), SARSCoV_Wuhan-HU-1 (GenBank Accession No. NC045512), SARSCoV_Unknown-UQ-581 (GenBank Accession No. MT412243), as well as any other subgroup 2b coronavirus now known (e.g., as can be found in the GenBank® Database) or later identified, and any combination thereof.

In some embodiments, the virus is a subgroup 2c coronavirus. Nonlimiting examples of a subgroup 2c coronavirus of any of the provided embodiments include Middle East respiratory syndrome coronavirus isolate Riyadh_2_2012 (GenBank Accession No. KF600652.1), Middle East respiratory syndrome coronavirus isolate Al-HasaJ 8J 013 (GenBank Accession No. F600651.1), Middle East respiratory syndrome coronavirus isolate Al-Hasa_17_2013 (GenBank Accession No. F600647.1), Middle East respiratory syndrome coronavirus isolate Al-Hasa_15_2013 (GenBank Accession No. F600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632.), Middle East respiratory syndrome coronavirus isolate Buraidah_1_2013 (GenBank Accession No. KF600630.1), Middle East respiratory syndrome coronavirus isolate Ffafr-Al-Batin_1_2013 (GenBank Accession No. F600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate RiyadhJ_2012 (GenBank Accession No. KF600612.1), Middle East respiratory syndrome coronavirus isolate AI-Hasa_3_2013 (GenBank Accession No. KF 186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. F186566.1), Middle East respiratory syndrome coronavirus isolate Al-Hasa__4_2013 (GenBank Accession No. KF 186564.1), Middle East respiratory syndrome coronavirus (GenBank Accession No. KF192507.1), Betacoronavirus England 1-N1 (GenBank Accession No. NC_019843), MERS-CoV_SA-Nl (GenBank Accession No. KC667074), following isolates of Middle East Respiratory Syndrome Coronavirus (GenBank Accession No: KF600656.1, GenBank Accession No; KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF 600641.1, GenBank Accession No: KF600633.1, GenBank Accession No: KF600629.1, GenBank Accession No: KF600617.1), Coronavirus Neoromicia/PML PHE1/RSA/201 1 GenBank Accession: KC869678.2, Bat Coronavirus Taper/CII_KSA_287/Bisha/Saudi Arabia/GenBank Accession No: KF493885.1,Bat coronavirus Rhhar/CII_KSAJ)03/Bisha Saudi Arabia/2013 GenBank\hat{ } Accession No: KF493888.1, Bat coronavirus Pikuh/CII\hat{ }KSA_001/Riyadh/Saudi Arabia/2013 GenBank_Accession No:KF493887.1, Bat coronavirus Rhhar/CII KSA_002/Bisha/Saudi Arabia/2013 GenBank Accession No: KF493886.1, Bat Coronavirus Rhhar/CIIJiSA_004/Bisha/Saudi Arabia 2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV133.2005 (GenBank Accession No. NCJ)08315), BtCoV.HKU5.5 (GenBank Accession No. EF065512); BtCoV.HKU5.1 (GenBank Accession No. NCJ)09020), BtCoV.HKU5.2 (GenBank Accession No. EF0655 IO), BtCoV.HKU5.3 (GenBank Accession No. EF06551 1), human betacoronavirus 2c Jordan-N3/2012 (GenBank Accession No. C776174.1; human betacoronavirus 2c EMC/2012, (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: C522086.1, GenBank Accession No: KC522085.1, GenBank Accession No: C522084.1, GenBank Accession No:KC522083.1, GenBank Accession No: KC522082.1, GenBank Accession No: KC522081, 1, GenBank Accession No: KC522080.1, GenBank Accession No: KC522079.1, GenBank Accession No: KC522078.1, GenBank Accession No: C522077.1, GenBank Accession No: KC522076.1, GenBank Accession No: KC522075.1, GenBank Accession No: KC522104.1, GenBank Accession No: C522104.1, GenBank Accession No: KC522103.1, GenBank Accession No: KC 522102.1, GenBank Accession No: C522101.1, GenBank Accession No: KC522100.1, GenBank Accession No: KC522099.1, GenBank Accession No: C522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC5221 19.1 GenBank Accession No: C5221 18.1 GenBank Accession No: C5221 17.1 GenBank Accession No: C5221 16.1 GenBank Accession No: C5221 15.1 GenBank Accession No: C5221 14.1 GenBank Accession No; KC5221 13,1 GenBank Accession No: C5221 12.1 GenBank Accession No: KC 522 1 1.1 GenBank Accession No: KC5221 10.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No:KC522046, 1, GenBank Accession No:KC522045.1, GenBank Accession No: KC522044.1, GenBank Accession No: KC522043. I, GenBank Accession No: KC522042.1, GenBank Accession No: KC522041.1, GenBank Accession No:KC522040.1 GenBank Accession No:KC522039.1, GenBank Accession No: C522038.1, GenBank Accession No: C522037.1, GenBank Accession No:KC522036.1, GenBank Accession No: C522048.1 GenBank Accession No:KC522047.1 GenBank Accession No:KC522046.1 GenBank Accession No:KC522045.1 GenBank Accession No: C522044,1 GenBank Accession No:KC522043.1 GenBank Accession No:KC522042.1 GenBank Accession No:KC522041 0.1 GenBank Accession No:KC522040, 1, GenBank Accession No:KC522039.1 GenBank Accession No:KC522038.1 GenBank Accession No:KC522037.1 GenBank Accession No:KC522036.1, GenBank Accession No:KC52206U GenBank Accession No:KC522060.1 GenBank Accession No:KC522059.1 GenBank Accession No:KC522058.1 GenBank Accession No:KC522057.1 GenBank Accession No: C522056.1 GenBank Accession No:KC522055, 1 GenBank Accession No: C522054.1 GenBank Accession No:KC522053, 1 GenBank Accession No:KC522052, 1 GenBank Accession No:KC522051.1 GenBank Accession No:KC522050.1 GenBank Accession No:KC522049.1 GenBank Accession No:KC522074.1, GenBank Accession No:KC522073.1 GenBank Accession No:KC522072.1 GenBank Accession No:KC522071.1 GenBank Accession No: C522070.1 GenBank Accession No:KC522069.1 GenBank Accession No:KC522068.1 GenBank Accession No:KC522067.1, GenBank Accession No:KC522066.1 GenBank Accession No:KC522065.1 GenBank Accession No:KC522064,1, GenBank Accession No: C522063.1, GenBank Accession No:KC522062.1), as well as any other subgroup 2c coronavirus now known (e.g., as can be found in the GenBank® Database) or later identified, and any combination thereof.

In some embodiments, the virus is a subgroup 2d coronavirus. Nonlimiting examples of a subgroup 2d coronavirus of any of the provided embodiments include BtCoV.HKU9.2 (GenBank Accession No. EF065514), BtCoV.HKU9.1 (GenBank Accession No. NCJ)09021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

In some embodiments, the virus is a subgroup 3 coronavirus. Nonlimiting examples of a subgroup 3 coronavirus of any of the provided embodiments include IBV.BeaudettelBV.p65 (GenBank Accession No. DQ001339), as well as any other subgroup 3 coronavirus now known (e.g., as can be found in the GenBank® Database) or later identified, and any combination thereof.

The coronaviruses in the respective subgroups 1a, 1b, 2a, 2b, 2c, 2d and 3 can be included in the methods and compositions of any of the provided embodiments in any combination, as would be well understood to one of ordinary skill in the art.

In some embodiments, the virus is a subgroup 2b coronavirus. In some embodiments, the virus is SARS CoV-2. The SARS-CoV-2 virus has been identified as the causative agent of the coronavirus disease first reported in China in 2019 (COVID-19) and was declared to be a global pandemic in March of 2020. This novel human coronavirus is a respiratory pathogen, known to be communicable via respiratory droplets and fomite transmission especially to the mucosa of the face and lungs. The latent period following exposure can be as along as 14-21 days. While many confirmed cases of SARS-CoV-2 infection result in asymptomatic presentation, death can result from severe disease. The related SARS-CoV-1 virus was identified as the causative agent for the first major SARS outbreak reported in East Asia in 2003. The Middle Eastern Respiratory Virus (MERS) was first reported as infected humans in 2012. It is thought that each of SARS CoV-1, SARS CoV-2, and MERS, are resultant from zoonotic transmission events via bats and possibly other livestock animals like swine and camelids. In some embodiments, the virus is SARS CoV-1. In some embodiments, the virus is MERS.

In some embodiments, the binding agents, particles, vehicles for delivery, or compositions areadministered in an effective amount to effect a therapeutic effect, such as to reduce or prevent a virus infection or severity of symptoms associated with a virus infection. Uses include uses of the polynucleotides, proteins (e.g. binding agents and particles), vehicles for delivery, or compositions in the preparation of a medicament in order to carry out such therapeutic methods. In some embodiments, the methods are carried out by administering a provided polynucleotide, protein (e.g. binding agents and particles), or vehicles for delivery, or compositions comprising the same, to the subject having or suspected of having a virus infection or being at risk of exposure to a virus causing a virus infection, e.g. a coronavirus infection, such as an infection with a CARS-COV-2 virus. In some embodiments, the methods thereby treat the virus infection in the subject.

It is therefore an object of the provided embodiments to provide methods of treatment, such as methods comprising compositions for delivery of polynucleotides or polypeptides (e.g. binding agents and particles), for the treatment of an infection, such as a coronavirus infection.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the vehicle particle may be administered alone or formulated as a pharmaceutical composition. In some embodiments, the vehicle particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a coronavirus infection). In some embodiments, the disease is a disease or disorder. In some embodiments, the disease is Severe Acute Respiratory Syndrome (SARS).

In some embodiments, the provided compositions are administered orally. Oral administration may include administration by inhalation, such as to produce an aerosol for delivery to the lungs. In some embodiments, the compositions may be administered using a dry-powder inhaler (DPI), pressurized metered-dose inhaler (pMDI) or a nebulizer. Any of the provided compositions formulated for oral administration, such as by inhalation, described herein can be administered in accord with the provided methods.

Aerosol delivery is an attractive approach because it is non-invasive and has the potential for delivering high concentrations of the therapeutic polynucleotide or polypeptide, such as those encoding a binding agent or particle. Aerosol delivery of nucleic acids to the lungs using viral vectors, polymers, surfactants, or excipients has been described. McDonald, et al., describes aerosol delivery of an adenoviral vector encoding the cystic fibrosis transmembrane conductance regulator protein (CFTR) to non-human primates (McDonald, et al., Human Gene Therapy 8:411-422 (1997)). Canonico, et al., describes the in vivo gene transfer of a plasmid containing recombinant human alpha 1-antitrpsin gene and a cytomegalovirus promoter complexed to cationic liposomes to the lungs by aerosol to rabbits (Canonico, et al., Am. J. Respir. Cell Mol. Biol, 10:24-29 (1994)). Stribling, et al., describes that the aerosol delivery of a chloramphenicol acetyltransferase reporter gene complexed to a cationic liposome carrier can produce CAT gene expression in mouse lungs (Stribling, et al., Proc. Natl Acad. Sci. USA 89:11277-11281 (1992)).

Massaro, et al., describes delivery of small inhibitory RNA molecules complexed to the lipoprotein pulmonary surfactant, known as surface active material or SAM, to the pulmonary alveoli in mice via liquid deposition into the nasal orifice (Massaro, et al., Am. J. Physiol. Lung Cell Mol. Physiol. 287:L1066-L1070 (2004)). U.S. Patent Application No. 2005/0008617 by Chen, et al., describes delivery of RNAi-inducing agents including short-interfering RNA (siRNA), short hairpin RNA (shRNA), and RNAi-inducing vectors complexed with cationic polymers, modified cationic polymers, lipids, and/or surfactants suitable for introduction into the lung. U.S. Patent Application No. 2003/0157030 by Davis, et al., describes administration of RNAi constructs such as siRNAs or nucleic acids that produce siRNAs complexed with polymers for nasal delivery. Any of such methods can be used in the provided embodiments.

In some embodiments, the provided polynucleotides, proteins (e.g. binding agents and particles), vehicles for delivery, or compositions containing any of the same, may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusible solutions or suspensions or suppositories or aerosols.

In some aspects, a “therapeutically effective time” refers to the period of time during which a pharmaceutically effective amount of a compound is administered, and that is sufficient to reduce one or more symptoms associated with a disease or disorder, such as SARS-coronavirus infection.

In some embodiments, the provided polynucleotides, proteins (e.g. binding agents and particles), vehicles for delivery, or compositions containing any of the same, may be administered before, concomitantly with, and/or after detection of symptoms of infection, such as with SARS-coronavirus. The term “concomitant” when in reference to the relationship between administration of a compound and symptoms means that administration occurs at the same time as, or during, manifestation of symptom associated with SARS-coronavirus infection. In some embodiments, the provided polynucleotides, proteins (e.g. binding agents and particles), vehicles for delivery, or compositions containing any of the same, provided herein may be administered before, concomitantly with, and/or after administration of another type of drug or therapeutic procedure.

In some embodiments, the composition may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the subject, etc.

As used herein, a “subject” is a mammal, such as a human or other animal, and typically is human. In some embodiments, the subject, e.g., patient, to whom the polynucleotides, vehicles for delivery, and compositions are administered, is a mammal, typically a primate, such as a human. In some embodiments, the primate is a monkey or an ape. The subject can be male or female and can be any suitable age, including infant, juvenile, adolescent, adult, and geriatric subjects.

As used herein, “treatment” (and grammatical variations thereof such as “treat” or “treating”) refers to complete or partial amelioration or reduction of a disease or condition or disorder, or a symptom, adverse effect or outcome, or phenotype associated therewith, such as is associated with coronavirus infection. Desirable effects of treatment include, but are not limited to, preventing occurrence or recurrence of infection, alleviation of symptoms, diminishment of any direct or indirect pathological consequences of the infection, preventing Severe Acute Respiratory Syndrome (SARS), decreasing the rate of disease progression, amelioration or palliation of the disease state, and remission or improved prognosis. The terms do not imply complete curing of a disease or complete elimination of any symptom or effect(s) on all symptoms or outcomes.

In some embodiments, the subject is known, suspected, or predicted to have been exposed to a SARS CoV-2 virus. In some embodiments, the subject has pneumonia, such as bilateral pneumonia. In some embodiments, the subject has ground-glass opacities, such as can be imaged with chest computed tomography (CT) scan.

In some embodiments, the subject is known, suspected, or predicted to have been exposed to a SARS CoV-2 virus. In some embodiments, the subject is known or suspected of having Severe Acute Respiratory Syndrome (SARS). SARS CoV-2 has a proposed staging system comprising three distinct disease phases. Mild or early infection is also known as Stage 1 coronavirus disease (COVID). Stage I COVID can be largely asymptomatic or presents with generally non-specific symptoms like malaise, cough, fever etc. Stage II COVID occurs with the establishment of pulmonary disease and/or pulmonary inflammation. In Stage IIa, patients do not display hypoxia associated with viral pneumonia (defined at PaO₂/FiO₂<300 mmHg). Stage IIb COVID is characterized by hypoxia and often necessitates mechanical ventilation. Stage III COVID is observed in the minority of patients and is strongly associated with mortality. In some aspects, Stage III COVID is characterized by systemic inflammation, such as is observed during a “cytokine storm”. There are no FDA approved treatments for COVID at any stage.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease.

“Preventing,” as used herein, includes providing prophylaxis with respect to the occurrence or recurrence of a disease in a subject that may be predisposed or exposed to the disease or disease causing agent but has not yet been diagnosed with the disease. In some embodiments, the provided the polynucleotides, vehicles for delivery, and compositions are used to delay development of a disease or to slow the progression of a disease.

An “effective amount”, e.g., a pharmaceutical formulation, or composition, including any containing provided polynucleotides, proteins (e.g. binding agents or particles), or vehicles for delivery, in the context of administration, refers to an amount effective, at dosages/amounts and for periods of time necessary, to achieve a desired result, such as a therapeutic or prophylactic result.

A “therapeutically effective amount”, e.g., a pharmaceutical formulation or composition, including any containing provided polynucleotides, proteins (e.g. binding agents or particles), or vehicles for delivery, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the polynucleotides administered. In some embodiments, the provided methods involve administering the provided polynucleotides, proteins (e.g. binding agents or particles), vehicles for delivery, or compositions containing any of the same, at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

In some embodiments, the provided polynucleotides, proteins (e.g. binding agents or particles), vehicles for delivery, or compositions containing any of the same, may ameliorate, or reduce, the severity of symptoms relative to baseline (Day 1 of administration of any of the provided polynucleotides, proteins (e.g. binding agents or particles), vehicles for delivery, or compositions containing any of the same, provided herein in any of Sections I, II, and III). In some embodiments, subjects are assessed for symptoms of coronavirus infection, including anyone of the following: fever, sore throat, cough, shortness of breath, myalgia. In some embodiments, the subjects self-assess. In some embodiments, subjects will be assessed on day 5 for when symptoms abate compared to baseline. In some embodiments, subjects will be assessed on day 7 for when symptoms abate compared to baseline. In some embodiments, subjects will be assessed on day 14 for when symptoms abate compared to baseline. In some embodiments, subjects will be assessed on day 21 for when symptoms completely abate to baseline. In some embodiments, subjects will be assessed on day 30 for when symptoms abate compared to baseline.

In some embodiments, the provided polynucleotides, proteins (e.g. binding agents or particles), vehicles for delivery, or compositions containing any of the same, may ameliorate, or reduce, the time to resolution of symptoms relative to baseline (Day 1 of administration of any of the polynucleotides, polypeptide, or vehicles provided herein in any of Sections I, II, and III). In some embodiments, subjects will be assessed on day 5 for when symptoms completely resolve compared to baseline. In some embodiments, subjects will be assessed on day 7 for when symptoms completely resolve compared to baseline. In some embodiments, subjects will be assessed on day 14 for when symptoms completely resolve compared to baseline. In some embodiments, subjects will be assessed on day 21 for when symptoms completely resolve compared to baseline. In some embodiments, subjects will be assessed on day 30 for when symptoms completely resolve compared to baseline.

In some embodiments, the provided polynucleotides, proteins (e.g. binding agents or particles), vehicles for delivery, or compositions containing any of the same, may ameliorate, or increase, the odds ratio for improvement on a 7-point ordinal scale on day 14. In some aspects, the odds ratio represents the odds of improvement in the ordinal scale between treatment groups. In some aspects, the ordinal scale is an assessment of the clinical status at a given day. The scale is set forth as follows: 1. Death 2. Hospitalized, on invasive mechanical ventilation or Extracorporeal Membrane Oxygenation (ECMO) 3. Hospitalized, on non-invasive ventilation or high flow oxygen devices 4. Hospitalized, requiring low flow supplemental oxygen 5. Hospitalized, not requiring supplemental oxygen—requiring ongoing medical care (coronavirus related or otherwise) 6. Hospitalized, not requiring supplemental oxygen—no longer required ongoing medical care 7. Not hospitalized.

In some embodiments, the provided polynucleotides, proteins (e.g. binding agents or particles), vehicles for delivery, or compositions containing any of the same, may ameliorate, or prevent, the need for an ER visit.

In some embodiments, the provided polynucleotides, proteins (e.g. binding agents or particles), vehicles for delivery, or compositions containing any of the same, may ameliorate, or reduce, the number of repeat ER visits following a first ER visit.

In some embodiments, the provided polynucleotides, proteins (e.g. binding agents or particles), vehicles for delivery, or compositions containing any of the same, may ameliorate, or reduce, the number of days in the intensive care unit (ICU). In some embodiments, the provided polynucleotides, proteins (e.g. binding agents or particles), vehicles for delivery, or compositions containing any of the same, may ameliorate, or reduce, the number of days a subject is placed on a ventilator, such as a mechanical ventilator.

In some embodiments, the provided polynucleotides, proteins (e.g. binding agents or particles), vehicles for delivery, or compositions containing any of the same, may ameliorate, or reduce, the number of days in the intensive care unit (ICU). In some embodiments, the provided polynucleotides, proteins (e.g. binding agents and particles), vehicles for delivery, or compositions containing any of the same, may ameliorate, or reduce, the number of days a subject is administered vasopressors.

In some embodiments, the provided polynucleotides, proteins (e.g. binding agents or particles), vehicles for delivery, or compositions containing any of the same, may ameliorate, or reduce, the number of days in the intensive care unit (ICU). In some embodiments, the provided polynucleotides, proteins (e.g. binding agents or particles), vehicles for delivery, or compositions containing any of the same, may ameliorate, or reduce, the number of days a subject is administered renal replacement therapy.

In some embodiments, the provided polynucleotides, proteins (e.g. binding agents or particles), vehicles for delivery, or compositions containing any of the same, may ameliorate, or reduce, the number of days needed for recovery from a coronavirus infection. In some aspects, subjects will be assed for recovery based on the following clinical criteria: normalization of pyrexia, respiratory rate and SPO₂, and relief of cough (where there are relevant abnormal symptoms at enrolment) that is maintained for at least 72 hours. In some embodiments, the provided polynucleotides, proteins (e.g. binding agents or particles), vehicles for delivery, or compositions containing any of the same, may ameliorate, or reduce, the number of days to resolution of pyrexia.

In some embodiments, the provided polynucleotides, proteins (e.g. binding agents or particles), vehicles for delivery, or compositions containing any of the same, may ameliorate, or reduce, number of adverse events as measured by Cancer Institute's Common Terminology Criteria for Adverse Events (NCI-CTCAE) v5.0, for at least one month, two months, three months, four months, five months, or six months following treatment.

In some embodiments, the provided polynucleotides, proteins (e.g. binding agents or particles), vehicles for delivery, or compositions containing any of the same, may ameliorate, or reduce, the number of days to resolution of cough. In some aspects, a subjects cough made be graded according to NCI-CTCAE v5.0, as set forth in Table 5 below.

TABLE 5
NCI-CTCAE v5.0 Cough Grading Scale

| | |
|---|---|
| Mild | Requires non-prescription treatment |
| Moderate | Require medication treatment, limits instrumental |
| | activities of daily living |
| Severe | Limits self-care activities of daily living |

In some embodiments, the provided polynucleotides, proteins (e.g. f binding agents or particles), vehicles for delivery, or compositions containing any of the same, may ameliorate, or reduce, the number of incidences of deterioration and/or aggravation of pneumonia. In some aspects, subjects will be assed for deteriorating/aggravated pneumonia based on the presence of at least one of the following clinical criteria: SPO₂≤93%, PaO₂/FiO₂≤300 mmHg, or distressed RR≥30/min without oxygen inhalation and requiring oxygen therapy or more advanced breath support.

In some aspects, subjects will be evaluated for QTc prolongation. A QT interval is a measurement of the electrical properties of the heart, assessed by an electrocardiogram. In some aspects, QT prolongation can result in tachycardia, such as Torsades de Pointes. In some aspects, a corrected QT (QT_{c}) of >500 ms confers a high risk of a cardiac event. In some aspects, an increase in a baseline QT_{c} of >60 ms confers a high risk of a cardiac event. In some aspects, a normal QT_{c} for an adult made is <430 ms. In some aspects, a normal QT_{c} for an adult female is <450 ms. In some aspects, a normal QT_{c} for an child under 15 is <440 ms.

In some embodiments, the provided polynucleotides, proteins (e.g. binding agents or particles), vehicles for delivery, or compositions containing any of the same, may ameliorate, or reduce, the severity of a symptoms relative to baseline (Day 1 of administration of any of the polynucleotides, polypeptide, or vehicles provided herein in any of Sections I, II, and III). In some embodiments, subjects are assessed for QT_{c} prolongation. In some embodiments, the subjects self-assess. In some embodiments, subjects will be assessed on day 5 for when symptoms abate compared to baseline as described above. In some embodiments, subjects will be assessed on day 7 for when symptoms abate compared to baseline. In some embodiments, subjects will be assessed on day 14 for when symptoms abate compared to baseline. In some embodiments, subjects will be assessed on day 21 for when symptoms completely abate to baseline. In some embodiments, subjects will be assessed on day 30 for when symptoms abate compared to baseline.

In some embodiments, the provided polynucleotides, proteins (e.g. binding agents or particles), vehicles for delivery, or compositions containing any of the same, may ameliorate, or reduce, viral load. In some embodiments, the provided polynucleotides, proteins (e.g. binding agents or particles), vehicles for delivery, or compositions containing any of the same, may prevent virologic failure, as defined as an increase in viral load of >0.5 log on two consecutive days, or >1 log increase in one day. In some aspects, wherein the viral load increase is not consistent with any baseline trend during the pre-treatment viral testing

In some of any embodiments, the provided polynucleotides, proteins (e.g. binding agents or particles), vehicles for delivery, or compositions containing any of the same, (e.g., any as described in Section II, a composition comprising the polynucleotides and polypeptides described in Sections I, and/or a composition comprising the vehicles described in Section III) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the vehicle particle composition comprises an exogenous polypeptide, such as encoding a binding agent or particle), the effect lasts for less than 1, 2, 3, 4, 5, 6, or 7 days, 2, 3, or 4 weeks, or 1, 2, 3, 6, or 12 months.

In some of any embodiments, the provided polynucleotides, proteins (e.g. binding agents or particles), vehicles for delivery, or compositions containing any of the same, described herein is delivered ex-vivo to a cell or tissue, e.g., a human cell or tissue. In some embodiments, the composition is delivered to an ex vivo tissue that is in an injured state (e.g., from trauma, disease, hypoxia, ischemia or other damage). In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the provided polynucleotides, proteins (e.g. binding agents or particles), vehicles for delivery, or compositions containing any of the same, described herein can be administered to a subject, e.g., a mammal, e.g., a human. In such embodiments, the subject may be at risk of, may have a symptom of, or may be diagnosed with or identified as having, a particular disease or condition (e.g., known or suspects of having a coronavirus infection). In some embodiments, the disease or condition is a respiratory syndrome, such as Severe Acute Respiratory Syndrome (SARS).

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Examples of lipid particles include solid particles such as nanoparticles, viral-derived particles or cell-derived particles. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity and include 10%-150% or more of the activity of a full-length or wild-type F protein or G protein. Examples of biologically active portions of F and G proteins include truncations of the cytoplasmic domain, e.g. truncations of up to 1, 2, 3, 4, 5, 6, 7, 8 9, 10, 11, 12, 13, 14, 15, 20,25, 30, 35 or more contiguous amino acids, see e.g. Khetawat and Broder 2010 Virology Journal 7:312; Witting et al. 2013 Gene Therapy 20:997-1005; published international; patent application No. WO/2013/148327.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (D)), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell.

As used herein, the term “specifically binds” to a target molecule, such as an antigen, means that a binding molecule, such as a an antibody or antigen-binding fragment thereof), reacts or associates more frequently, more rapidly, with greater duration and/or with greater affinity with a particular target molecule than it does with alternative molecules. A binding molecule, such as an antibody or antigen-binding fragment thereof, “specifically binds” to a target molecule if it binds with greater affinity, avidity, more readily, and/or with greater duration than it binds to other molecules. It is understood that a binding molecule, such as an antibody or antigen-binding fragment thereof), that specifically binds to a first target may or may not specifically bind to a second target. As such, “specific binding” does not necessarily require (although it can include) exclusive binding.

As used herein, the term “neutralizing,” or “neutralization” or grammatical variations thereof in relation to the binding agents refer to those that contain a binding domain (e.g. antibody or antigen-binding fragment thereof) that blocks or reduces at least one activity of a virus having exposed on its surface a viral surface protein to which the binding domain (e.g. antibody or antigen-binding fragment thereof) specifically binds. For example, neutralization can be achieved by inhibiting the attachment or adhesion of the virus to a target cell surface, e.g., by a binding domain (e.g. an antibody or antigen-binding fragment thereof) that binds directly to, or close by, the site responsible for the attachment or adhesion of the virus. Neutralizing activity includes activity to inhibit a virus from replication. A neutralizing activity may be measured in vitro and/or in vivo.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Amino acid substitutions may be introduced into an antibody of interest and the products screened for a desired activity, for example, retained/improved binding. Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a “vehicle” refers to a biological carrier for delivering genes or proteins to cells to facilitate their recognition or uptake by cells. Examples of delivery vehicles include, but are not limited to, lipid and non-lipid particles, such as virus or virus like particles, liposomes, microparticles, nanoparticles, nanogels, dendrimer or dendrisomes.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of any of the provided embodiments with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### VI. Exemplary Embodiments

Among the provided embodiments are:

1. A binding agent, comprising: (i) at least one binding domain that binds to a viral protein exposed on the surface of a virus, and (ii) a modified Fe domain, wherein the binding agent is capable of neutralizing the virus and exhibits reduced pro-inflammatory activity compared to an unmodified Fc domain.

2. A binding agent, comprising: (i) at least one binding domain that binds to a viral protein exposed on the surface of a virus, and (ii) an Fc domain with reduced pro-inflammatory activity as compared to an IgG1 Fc domain, wherein the binding agent is capable of neutralizing the virus.

3. The binding agent of embodiment 2, wherein the Fc domain is an IgG2 or IgG4 Fc domain.

4. The binding agent of embodiment 1, wherein the modified Fc exhibits reduced binding to an Fc activating receptor.

5. The binding agent of embodiment 1, wherein the modified Fc exhibits increased binding to an Fc inhibitory receptor compared to a wild-type Fc domain.

6. A binding agent, comprising (i) at least one binding domain that binds to a surface exposed viral protein, and (ii) a modified Fc domain wherein the modified Fc domain has decreased binding to at least one Fc activating receptor family member compared to the wild-type Fc domain.

7. The binding agent of embodiment 4 or embodiment 6, wherein the Fc activating receptor is Fc gamma receptor I (FcγRI), Fc gamma receptor IIA (FcγRIIA) or Fc gamma receptor III (FcγRIII).

8. The binding agent of any of embodiments 1-7, wherein the binding agent is capable of forming an immune complex with decreased pro-inflammatory activity compared to an immune complex formed with a binding agent comprising the at least one binding domain and a wild-type Fc domain.

9. The binding agent of any of embodiments 1, 4, or 6-8, wherein the modified Fc domain comprises an amino acid substitution selected from, Ser228Pro, Glu233Pro, Leu234Ala, Leu234Glu, Leu235Ala, Leu235Glu, Leu235Phe, Gly236Arg, Gly237Ala, Pro238Ser, Asp265Ala, His268Ala, His268Gln, Ser288Pro, Asn297Ala, Asn297Gly, Asn297Gln, Val309Leu, Gly318Ala, Leu328Arg, Pro329Gly, Ala330Ser, and Pro331Ser, each based on EU numbering, or combinations of any of the foregoing.

10. The binding agent of any of embodiments 1-9, wherein the modified Fe domain comprises a Leu235Glu substitution based on EU numbering.

11. The binding agent of any of embodiments 1-9, wherein the modified Fe domain comprises a Leu234Ala substitution based on EU numbering and Leu235Ala substitution based on EU numbering.

12. The binding agent of any of embodiments 1-9, wherein the modified Fe domain comprises a Ser288Pro substitution based on EU numbering and Leu235Glu substitution based on EU numbering.

13. The binding agent of any of embodiments 1-9, wherein the modified Fe domain comprises a Leu234Ala substitution based on EU numbering, Leu235Ala substitution based on EU numbering, and Pro329Gly substitution based on EU numbering.

14. The binding agent of any of embodiments 1-9, wherein the modified Fe domain comprises a Pro331Ser substitution based on EU numbering, Leu234Glu substitution based on EU numbering, and Leu235Phe substitution based on EU numbering.

15. The binding agent of any of embodiments 1-9, wherein the modified Fe domain comprises a Asp265Ala substitution based on EU numbering.

16. The binding agent of any of embodiments 1-9, wherein the modified Fe domain comprises a Gly237Ala substitution based on EU numbering.

17. The binding agent of any of embodiments 1-9, wherein the modified Fe domain comprises a Gly318Ala substitution based on EU numbering.

18. The binding agent of any of embodiments 1-9, wherein the modified Fe domain comprises a Glu233Pro substitution based on EU numbering.

19. The binding agent of any of embodiments 1-9, wherein the modified Fe domain comprises a Gly236Arg substitution based on EU numbering, Leu328Arg substitution based on EU numbering, and Pro329Gly substitution based on EU numbering.

20. The binding agent of any of embodiments 1-9, wherein the modified Fe domain comprises a His268Gln substitution based on EU numbering, Val309Leu substitution based on EU numbering, and Ala330Ser substitution based on EU numbering, and/or Pro331Ser substitution based on EU numbering.

21. The binding agent of any of embodiments 1-9, wherein the modified Fe domain comprises a Leu234Ala substitution based on EU numbering, Leu235Ala substitution based on EU numbering, Gly237Ala substitution based on EU numbering, Pro238Ser substitution based on EU numbering, His268Ala substitution based on EU numbering, Ala330Ser substitution based on EU numbering, and Pro331Ser substitution based on EU numbering.

22. The binding agent of any of embodiments 1-9, wherein the modified Fe domain comprises a Asn297Ala substitution based on EU numbering, Asn297Gly substitution based on EU numbering, or Asn297Gln substitution based on EU numbering.

23. The binding agent of any of embodiments 1-9, wherein the modified Fe domain comprises a Ser228Pro substitution based on EU numbering, Phe234Ala substitution based on EU numbering, and Leu235Ala substitution based on EU numbering.

24. A binding agent, comprising (i) at least one binding domain that binds to a viral protein exposed on the surface of a virus, and (ii) a modified Fe domain wherein the modified Fe domain has increased binding to an inhibitory Fc receptor compared to the wild-type Fe domain.

25. The binding agent of embodiment 5 or 24, wherein the inhibitory Fc receptor is an FcγRIIB, optionally wherein the FcRIIB is FcγRIIB1 or FcγRIIB2.

26. The binding agent of any of embodiments 1, 2, 5, or 24-25, wherein the binding agent is capable of forming an immune complex with increased anti-inflammatory activity compared to an immune complex formed with a binding agent comprising the at least one binding domain and a wild-type Fe domain.

27. The binding agent of any of embodiments 1, 2, 5, or 24-25, wherein the binding agent is capable of forming an immune complex with decreased inflammatory activity compared to an immune complex formed with a binding agent comprising the at least one binding domain and a wild-type Fe domain.

28. The binding agent of any of embodiments 1, 5, or 24-27, wherein the modified Fe domain comprises an amino acid substitution selected from, Phe241Ala, Ser267Glu, His268Phe, Leu328Phe, Ser324Thr, Pro238Asp, Leu328Glu, Ser239Asp, Ile332Glu, Gly236Ala each based on EU numbering, or combinations of any of the foregoing.

29. The binding agent of any of embodiments 1, 5, or 24-28, wherein the modified Fe domain comprises a Ser267Glu substitution based on EU numbering and His268Phe substitution based on EU numbering, and Ser324Thr substitution based on EU numbering.

30. The binding agent of any of embodiments 1, 5, or 24-29, wherein the modified Fe domain comprises a Ser267Glu substitution based on EU numbering and Leu328Phe substitution based on EU numbering.

31. The binding agent of any of embodiments 1, 5, or 24-30, wherein the modified Fc domain comprises a Pro238Asp substitution based on EU numbering.

32. The binding agent of any of embodiments 1, 5, or 24-31, wherein the modified Fc domain comprises a Leu328Glu substitution based on EU numbering.

33. The binding agent of any of embodiments 1, 5, or 24-32, wherein the modified Fc domain comprises a Ser239Asp substitution based on EU numbering and Ile332Glu substitution based on EU numbering.

34. The binding agent of any of embodiments 1, 5, or 24-33, wherein the modified Fc domain comprises a Ser239Asp substitution based on EU numbering and Ile332Glu substitution based on EU numbering, and Gly236Ala substitution based on EU numbering.

35. The binding agent of any of embodiments 1, 5, or 24-34, wherein the modified Fc domain comprises a Ser267Glu substitution based on EU numbering.

36. The binding agent of any of embodiments 1, 5, or 24-35, wherein the modified Fc domain comprises a E233D substitution based on EU numbering.

37. The binding agent of any of embodiments 1, 5, or 24-36, wherein the modified Fc domain comprises a G237D substitution based on EU numbering.

38. The binding agent of any of embodiments 1, 5, or 24-37, wherein the modified Fc domain comprises a H268D substitution based on EU numbering.

39. The binding agent of any of embodiments 1, 5, or 24-38, wherein the modified Fc domain comprises a P271G substitution based on EU numbering.

40. The binding agent of any of embodiments 1, 5, or 24-39, wherein the modified Fc domain comprises a A330R substitution based on EU numbering.

41. The binding agent of any of embodiments 1, 5, or 24-40, wherein the modified Fc domain comprises a E233D substitution based on EU numbering and a A330R substitution based on EU numbering.

42. The binding agent of any of embodiments 1, 5, or 24-41, wherein the modified Fc domain comprises a E233D substitution based on EU numbering, a P271G substitution based on EU numbering, and a A330R substitution based on EU numbering.

43. The binding agent of any of embodiments 1, 5, or 24-42, wherein the modified Fc domain comprises a G237D substitution based on EU numbering, a H268D substitution based on EU numbering, and a P271G substitution based on EU numbering.

44. The binding agent of any of embodiments 1, 5, or 24-43, wherein the modified Fc domain comprises a G237D substitution based on EU numbering, a P271G substitution based on EU numbering, and a A330R substitution based on EU numbering.

45. The binding agent of any of embodiments 1, 5, or 24-44, wherein the modified Fc domain comprises a E233D substitution based on EU numbering, a H268D substitution based on EU numbering, a P271G substitution based on EU numbering, and a A330R substitution based on EU numbering.

46. The binding agent of any of embodiments 1, 5, or 24-45, wherein the modified Fc domain comprises a G237D substitution based on EU numbering, a H268D substitution based on EU numbering, a P271G substitution based on EU numbering, and a A330R substitution based on EU numbering.

47. The binding agent of any of embodiments 1, 5, or 24-46, wherein the modified Fc domain comprises a E233D substitution based on EU numbering, a G237D substitution based on EU numbering, a H268D substitution based on EU numbering, a P271G substitution based on EU numbering, and a A330R substitution based on EU numbering.

48. The binding agent of any of embodiments 1-47, wherein the modified Fe domain comprises one or more amino acid substitutions compared to wildtype Fe domain.

49. The binding agent of any of embodiments 1-48, wherein the wildtype Fe domain is a wildtype IgG1.

50. The binding agent of any of embodiments 1-49, wherein the wildtype Fe domain comprises the sequence of amino acids set forth in SEQ ID NO 1.

51. The binding agent of any of embodiments 1-49 wherein the modified Fe domain comprises a sequence of amino acids that exhibits at least 85%, at least 90%, at least 92%, at least 95%, at least 97% sequence identity to SEQ ID NO 1.

52. The binding agent of any of embodiments 1-49, wherein the wildtype Fe domain is a wildtype IgG2.

53. The binding agent of any of embodiments 1-49, wherein the wildtype Fe domain comprises the sequence of amino acids set forth in SEQ ID NO 2.

54. The binding agent of any of embodiments 1-49 wherein the modified Fe domain comprises a sequence of amino acids that exhibits at least 85%, at least 90%, at least 92%, at least 95%, at least 97% sequence identity to SEQ ID NO 2.

55. The binding agent of any of embodiments 1-48, wherein the wildtype Fe domain is a wildtype IgG4.

56. The binding agent of any of embodiments 1-48, wherein the wildtype Fe domain comprises the sequence of amino acids set forth in SEQ ID NO 3.

57. The binding agent of any of embodiments 1-48 wherein the modified Fc domain comprises a sequence of amino acids that exhibits at least 85%, at least 90%, at least 92%, at least 95%, at least 97% sequence identity to SEQ ID NO 3.

58. The binding agent of any of embodiments 1-48 wherein the modified Fc domain comprises a sequence of amino acids that exhibits at least 85% sequence identity to any of SEQ ID NO 1-3.

59. The binding agent of any of embodiments 1-58, wherein the at least one binding domain and modified Fc domain are directly linked.

60. The binding agent of any of embodiments 1-58, wherein the at least one binding domain and modified Fc domain are indirectly linked via a linker.

61. The binding agent of embodiment 60, wherein the linker is a peptide linker.

62. The binding agent of embodiments 60-61, wherein the peptide linker is (GₘS)n (SEQ ID NO: 4), wherein each of m and n is an integer between 1 to 4, inclusive.

63. The binding agent of any of embodiments 1-62, wherein the at least one binding domain is at least two binding domains.

64. The binding agent of embodiment 63, wherein the at least two binding domains bind at least two distinct epitopes of the viral protein.

65. The binding agent of any of embodiments 1-64, wherein the at least two binding domains are directly linked.

66. The binding agent of any of embodiments 1-64, wherein the at least two binding domains are indirectly linked via a linker.

67. The binding agent of embodiment 66, wherein the linker is a peptide linker.

68. The binding agent of embodiment 66 or embodiment 67, wherein the peptide linker is (GₘS)ₙ(SEQ ID NO: 4), wherein each of m and n is an integer between 1 to 4, inclusive.

69. The binding agent of any of embodiments 1-68, wherein the viral protein is a viral receptor.

70. The binding agent of any of embodiments 1-69, wherein the virus is an RNA virus.

71. The binding agent of any of embodiments 1-70, wherein the virus is an orthomyxovirus, optionally wherein the virus is an influenza virus.

72. The binding agent of any of embodiments 1-70, wherein the virus is a paramyxovirus.

73. The binding agent of embodiment 72, wherein the virus is Respiratory Syncytial Virus (RSV).

74. The binding agent of embodiment 72, wherein the virus is Measles morbillivirus (MeV).

75. The binding agent of any of embodiments 1-70, wherein the virus is a coronavirus.

76. The binding agent of embodiment 75, wherein the virus is Severe Acute Respiratory Syndrome (SARS) CoV-2.

77. The binding agent of embodiment 76, wherein the virus is SARS CoV-1.

78. The binding agent of embodiment 77, wherein the virus is Middle Eastern Respiratory Syndrome Virus (MERS-V).

79. The binding agent of any of embodiments 1-78, wherein the binding agent is a dimer.

80. The binding agent of any of embodiments 1-79, wherein the binding agent is capable of neutralizing the virus.

81. The binding agent of any of embodiments 1-80, wherein the binding agent reduces or prevents viral attachment to a host cell.

82. The binding agent of any of embodiments 1-81, wherein the at least one binding domain comprises an antigen-binding fragment of an antibody that specifically binds the viral protein.

83. The binding agent of embodiment 82, wherein the antigen-binding fragment comprises a variable heavy chain (VH) and a variable light chain (VL).

84. The binding agent of embodiment 82 or embodiment 83, wherein the antigen-binding fragment is selected from among a Fab fragment, F(ab′)₂ fragment, Fab′ fragment, Fv fragment.

85. The binding agent of any of embodiments 1-84, wherein the at least one binding domain specifically binds the S (spike) glycoprotein of a SARS virus.

86. The binding agent of any of embodiments 1-85, wherein the at least one binding domain is an antigen-binding fragment of an antibody selected from among STI-1499, STI-4398, REGN10933, REGN10987, REGN-COV2, JS016, LY-CoV555, LY-3819253, TB181-8, TB181-28, TB181-36, BGB-DXP593, TY027, CT-P59, BRII-196, BRII-198, SCTA01, MW33, AZD8895, AZD1061, HLX70, 15G11, 18F4, 1E5, 1G3, 21C3, 22d(23D11, 26E2, 29F7, 3B3, 3F2, D59047-11955, D70678-12637-S1, D70678-12799-S1, D70678-13531-S1, D70678-13576-S1, D70678-14004-S2, D70678-14027-S2, D70678-2155-S1, D70678-2743-S1, and D70678-5521-S2.

87. The binding agent of any of embodiments 1-82, wherein the at least one binding domain is a single domain antibody (sdAb), optionally wherein the at least one binding domain is TB201-1, TB202-3, TB202-63.

88. The binding agent of any of embodiments 1-82, wherein the at least one binding domain is an antibody mimetic, optionally selected from Designed Ankyrin Repeat Protein (DARPin), adnectins, or an antigen-binding fibronectin type III (Fn3) scaffold.

89. A particle, comprising (i) the binding agent of any of embodiments 1-88 and (ii) a viral protein capable of being bound by the at least one binding domain of the binding agent.

90. The particle of embodiment 89, wherein the viral protein is a purified viral protein.

91. The particle of embodiment 89, wherein the viral protein is a recombinant viral protein.

92. The particle of any of embodiments 89-91, wherein the viral protein is the S (spike) glycoprotein of a SARS virus

93. A nucleic acid molecule, encoding the binding agent of any of embodiments 1-88.

94. The nucleic acid molecule of embodiment 93, wherein the at least one binding domain of the binding agent comprises a variable heavy chain (VH) and a variable light chain (VL), wherein the nucleic acid comprises a first sequence encoding the VH chain and the modified Fc and a second sequence encoding the VL chain and wherein the first and second sequence are separated by a multicistronic element.

95. The nucleic acid of embodiment 94, wherein the multicistronic element(s) comprises a sequence encoding a ribosome skip element selected from among a T2A, a P2A, a E2A or a F2A or an internal ribosome entry site (IRES).

96. The nucleic acid of embodiment 94 or embodiment 95, wherein the first sequence and the second sequence are operably connected to the same promoter.

97. The nucleic acid molecule of any of embodiments 93-96 wherein the nucleic acid molecule is mRNA.

98. A cell, comprising nucleic acid of any of embodiments 93-96. 99. A vector, comprising the nucleic acid molecule of any of embodiments 93-98. 100. The vector of embodiment 99, wherein the vector is a viral vector or viral like particle.

101. The vector of embodiment 99, wherein the vector is derived from an adenovirus.

102. The vector of embodiment 99, wherein the viral vector is derived from an Adeno-associated virus (AAV).

103. The vector of embodiment 102, wherein the AAV is of serotype 1, 2, 5, 6, 8 or 9.

104. The vector of embodiment 102 or embodiment 103, wherein the AAV is of serotype 8.

105. The vector of embodiment 102 or embodiment 103, wherein the AAV is of serotype 6.

106. The vector of embodiment 105, wherein the AAV is of serotype 6.2. 107. The vector of embodiment 99, wherein the viral vector is derived from a lentivirus.

108. The vector of embodiment 107, wherein the lentivirus is Human Immunodeficiency Virus-1 (HIV-1).

109. The vector of any of embodiments 99-108, wherein the viral vector or viral-like particle comprises a fusogen.

110. The vector of any of embodiments 99-109, wherein the vector is a lipid particle, wherein the lipid particle comprises (i) a lipid bilayer enclosing a lumen, and (ii) a fusogen, wherein the fusogen is embedded in the lipid bilayer.

111. The vector of embodiment 110, wherein the lipid bilayer is derived from a membrane of a host cell used for producing a virus or virus-like particle.

112. The vector of embodiment 110 or embodiment 111, wherein the lipid bilayer is derived from a membrane of a host cell used for producing a virus-like particle, wherein the virus-like particle is replication defective.

113. The vector of any of embodiments 110-112, wherein the fusogen is a viral fusogen selected from a Class I viral membrane fusion protein, a Class II viral membrane protein, a Class II viral membrane fusion protein, a viral membrane glycoprotein, or a viral envelope protein.

114. The vector of any of embodiments 110-113, wherein the fusogen is a vesicular stomatitis virus envelope glycoprotein (VSV-G).

115. The vector of any of embodiments 110-113, wherein the fusogen is a baboon endogenous virus (BaEV) envelope glycoprotein.

116. The vector of any of embodiments any of embodiments 110-113, wherein the fusogen is a syncytin.

117. The vector of any of embodiments 110-113, wherein the fusogen is from a coronavirus.

118. The vector of any of embodiments any of embodiments 110-113 and 117, wherein the fusogen is a Severe Acute Respiratory Syndrome (SARS) coronavirus 1 (SARS CoV-1) spike glycoprotein.

119. The vector of any of embodiments any of embodiments 110-113 and 117, wherein the fusogen is a Severe Acute Respiratory Syndrome (SARS) coronavirus 2 (SARS CoV-2) spike glycoprotein.

120. The vector of any of embodiments any of embodiments 110-113, 118 and 119, wherein the fusogen is an alpha coronavirus CD13 protein.

121. The vector of any of embodiments 110-112, wherein the fusogen comprises an F protein molecule or a biologically active portion thereof from a Paramyxovirus and/or a glycoprotein G (G protein) or a biologically active portion thereof from a Paramyxovirus.

122. The vector of any of embodiments 110-112, wherein the fusogen is derived from an F protein molecule or a biologically active portion thereof from a Paramyxovirus and/or a glycoprotein G (G protein) or a biologically active portion thereof from a Paramyxovirus.

123. The vector of embodiment 121 or embodiment 122, wherein the Paramyxovirus is a henipavirus.

124. The vector of any of embodiments 121-123, wherein the Paramyxovirus is Nipah virus.

125. The vector of any of embodiments 121-124, wherein the Paramyxovirus is Hendra virus.

126. The vector of any one of embodiments 110-125, wherein the fusogen is a re-targeted fusogen comprising a targeting moiety that binds to a molecule on a target cell.

127. The vector of embodiment 126, wherein the targeting moiety is a Design ankyrin repeat proteins (DARPin), a single domain antibody (sdAb), a single chain variable fragment (scFv), or an antigen-binding fibronectin type III (Fn3) scaffold.

128. The vector of embodiment 126 or embodiment 127, wherein the target cell is known or suspected of being infected by a coronavirus.

129. The vector of any of embodiments 126-128, wherein targeting moiety binds a receptor of a coronavirus.

130. The vector of any of embodiments 126-129, wherein the targeting moiety binds angiotensin-converting enzyme 2 (ACE2).

131. The vector of embodiment 126 or embodiment 127, wherein the target cell is a B lymphocyte.

132. The vector of any of embodiment 131, wherein the targeting moiety binds to human CD20. 133. The vector of any one of embodiments 121-132, wherein the fusogen is modified to reduce its native binding tropism.

134. The vector of any of embodiments 121-133, wherein the G protein or the biologically active portion thereof is a mutant NiV-G protein that exhibits reduced binding to Ephrin B2 or Ephrin B3.

135. The vector of embodiment 134, wherein the mutant NiV-G protein comprises one or more amino acid substitutions corresponding to amino acid substitutions selected from the group consisting of E501A, W504A, Q530A and E533A with reference to numbering set forth in SEQ ID NO:5.

136. The vector of any of embodiments 134 or 135, wherein the mutant NiV-G protein has the amino acid sequence set forth in SEQ ID NO: 34 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:34.

137. The vector of any of embodiments 134-136, wherein the mutant NiV-G protein has the amino acid sequence set forth in SEQ ID NO: 35 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:35.

138. The vector of any of embodiments 121-133, wherein the NiV-F protein is a biologically active portion thereof that has a 20 amino acid truncation at or near the C-terminus of the wild-type NiV-F protein (SEQ ID NO:19).

139. The vector of embodiment 138, wherein the NiV-F protein has an amino acid sequence set forth in SEQ ID NO:32 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 32.

140. The vector of any of embodiments 121-133, wherein the NiV-F protein is a biologically active portion thereof that has a 22 amino acid truncation at or near the C-terminus of the wild-type NiV-F protein (SEQ ID NO:37).

141. The vector of embodiment 140, wherein the NiV-F protein has an amino acid sequence set forth in SEQ ID NO:36 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36.

142. The vector of embodiment 140 or embodiment 141, wherein the NiV-F protein has an amino acid sequence set forth in SEQ ID NO:38 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 38.

143. The vector of any of embodiments 121-142, wherein the NiV-F protein comprises a point mutation on an N-linked glycosylation site.

144. The vector of any of embodiments 121-140 and 143, wherein the NiV-F protein is a biologically active portion thereof that comprises:
i) a 20 amino acid truncation at or near the C-terminus of the wild-type NiV-F protein (SEQ ID NO:36); and
ii) a point mutation on an N-linked glycosylation site.

145. A method of producing a binding agent, comprising introducing the nucleic acid molecule of any of embodiments 93-98 or vector of any of embodiments 99-144 into a host cell under conditions to express the binding agent in the cell.

146. The method of embodiment 145, further comprising isolating or purifying the binding agent from the cell.

147. A method of forming an immune complex, the method comprising administering the one or more binding agent of any of embodiments 1-88 to a subject known or suspected of having a viral infection, wherein an immune complex comprising the administered one or more binding agent is formed in the subject.

148. The method of embodiment 147, wherein the immune complex further comprises at least one endogenous antibody against a viral protein exposed on the surface of the virus.

149. The method of any of embodiments 147 or 148, wherein the one or more binding agent and the at least one endogenous antibody bind the same viral protein.

150. The method of any of embodiments 147-149, wherein the one or more binding agent and the at least one endogenous agent bind the same epitope of the viral protein.

151. The method of any of embodiments 147-150, wherein the one or more binding agent and the at least one endogenous agent bind a distinct epitope of the viral protein.

152. The method of any of embodiments 147-151, wherein the one or more binding agent and the at least one endogenous agent bind an overlapping epitope of the viral protein.

153. A composition comprising an immune complex comprising: (i) the binding agent of any of embodiments 1-88 and (ii) a surface exposed viral protein bound by the at least one binding domain of the binding agent.

154. The composition of embodiment 153, wherein the immune complex comprises two or more binding agents.

155. The composition of embodiments 153-154, wherein the at least two binding domains bind a distinct epitope of the viral protein.

156. The composition of embodiments 153-155, wherein the immune complex further comprises endogenous binding domains, optionally from an endogenous antibody and/or antibodies.

157. The composition of any of embodiments 153-156, wherein the at least two binding domains and endogenous binding domains bind the same epitope of the viral protein.

158. The composition of embodiments 153-156, wherein the at least two binding domains and endogenous binding domains bind a distinct epitope of the viral protein.

159. The composition of embodiments 153-156, wherein the at least two binding domains and endogenous binding domains bind an overlapping epitope of the viral protein.

160. A pharmaceutical composition, comprising the binding agent of any of embodiments 1-23, the nucleic acid of any of embodiments 93-97, the cell of embodiment 98, and/or the vector of any of embodiments 99-144.

161. A pharmaceutical composition, comprising the binding agent of any of embodiments 23-88, the nucleic acid of any of embodiments 93-97, the cell of embodiment 98, and/or the vector of any of embodiments 99-144.

162. A pharmaceutical composition, comprising the particle of any of embodiments 89-92.

163. A pharmaceutical composition, comprising the cell of embodiment 98.

164. A pharmaceutical composition, comprising (i) the binding agent of any of embodiments 1-88 and (ii) a recombinant viral protein capable of being bound by the at least one binding domain of the binding agent.

165. The pharmaceutical composition of any of embodiments 153-164, comprising a pharmaceutically acceptable excipient.

166. The pharmaceutical composition of any of embodiments 153-165 that is sterile.

167. A method of reducing inflammation in response to a viral infection in a subject, the method comprising administering, to a subject known or suspected of having a virus infection, a therapeutically effective amount of a binding agent of any one of embodiments 1-88, the particle of any of embodiments 89-92, the nucleic acid of any of embodiments 93-97, the cell of embodiment 98, or the vector of any of embodiments 99-144.

168. A method of reducing inflammation in response to a viral infection in a subject, the method comprising administering to a subject known or suspected of having a virus infection, a therapeutically effective amount of the pharmaceutical composition of any of embodiments 160-166 to the subject.

169. A method of reducing inflammation in response to a viral infection in a subject, the method comprising administering to a subject known or suspected of having a virus infection, (i) a therapeutically effective amount of the pharmaceutical composition of embodiment 160 (ii) a recombinant viral protein capable of being bound by the at least one binding domain of the binding agent.

170. A method of reducing inflammation in response to a viral infection in a subject, the method comprising administering to a subject known or suspected of having a virus infection, (i) a therapeutically effective amount of the pharmaceutical composition of embodiment 161 (ii) a recombinant viral protein capable of being bound by the at least one binding domain of the binding agent.

171. The method of any of embodiments 167-170, wherein the inflammation comprises lymphocytic accumulation in the lung, lymphocytic proliferation in the lung, peripheral blood lymphopenia, pro-inflammatory cytokine production, or combinations of any of the foregoing.

172. The method of embodiment 171, wherein the pro-inflammatory cytokine is selected from the group consisting of: MCP-1, IL-8, IL-1β, IFN-γ, IP-10, IL-4, IL-1β, IL-2, IL-7, GCSF, MIP-1A, and TNF-α.

173. The method of any of embodiments 169 or 170, wherein the pharmaceutical composition and recombinant viral protein are administered concurrently.

174. The method of any of embodiments 169 or 170, wherein the pharmaceutical composition and recombinant viral protein are administered sequentially, optionally wherein the recombinant viral protein is first administered.

175. The method of any of embodiments 169 or 170, wherein the pharmaceutical composition and recombinant viral protein are administered sequentially, optionally wherein the pharmaceutical composition is first administered.

176. A method of promoting inhibitory immune complex function, comprising administering therapeutically effective amount of the pharmaceutical composition in any of embodiments 160, 162-166.

177. A method of promoting inhibitory immune complex function, comprising administering therapeutically effective amount of the binding agent of any one of embodiments 24-88, the particle of any of embodiments 89-92, the nucleic acid of any of embodiments 93-97, the cell of embodiment 98, the vector of any of embodiments 99-144.

178. The method of any of embodiments 176-177, wherein inhibitory immune complex function comprises diminished antigen uptake, diminished antigen presentation, reduced cellular activation, reduced antibody secretion, production of anti-inflammatory cytokines, or combinations of any of the foregoing.

179. A method of reducing activating immune complex function, comprising administering therapeutically effective amount of the pharmaceutical composition in any of embodiments 161-166.

180. A method of reducing activating immune complex function, comprising administering a therapeutically effective amount of the binding agent of any one of embodiments 1-21, the particle of any of embodiments 89-92, the nucleic acid of any of embodiments 93-97, the cell of embodiment 98, the vector of any of embodiments 99-144.

181. The method of any of embodiments 179-180, wherein activating immune complex function comprises antibody-dependent cell mediated cytotoxicity (ADCC), antibody dependent enhancement (ADE), release of inflammatory mediators, production of pro-cytokines, phagocytosis, or combinations of any of the foregoing.

182. The method of any of embodiments 167-181, wherein the method further comprises treatment of a viral infection in a subject.

183. The method of embodiment 182, wherein the viral infection is an infection caused by a virus with a surface exposed viral protein recognized by the at least one binding domain of the binding agent.

184. The method of any of any of embodiment 183, wherein the virus is an RNA virus.

185. The method of any of embodiments 183 or 184, wherein the virus selected from SARS-CoV-1, SARS-CoV-2, MERS, RSV, influenza viruses, and measles virus.

### VII. Examples

The following example is included for illustrative purposes only and is not intended to limit the scope of the invention.

An AAV2.5T vector (Excoffon et al., 2009, Proc. Natl. Acad. Sci. U.S.A, 106:3865-70) expressing an binding agent consisting of (i) a humanized llama anti-SARS-COV2 spike protein, (ii) a human IgG1 hinge domain, and (iii) a human IgG1 Fc domain with P238D, E233D, G237D, H268D, P271G, and A330R substitutions (V12; Mimoto et al., Protein Eng Des Sel. 26(10):589-598, 2013) under control of a hybrid promoter having a human cytomegalovirus (CMV) enhancer and the elongation factor 1a promoter (hCEF) is administered by inhalation to patients having been infected with SARS-CoV-2. The AAV vector drives expression of the binding agent by infected cells.

The patients are evaluated for inflammation through 28 days following treatment via (i) pulmonary function tests, such as pulse oximetry or an exhaled nitric oxide test (ii) in situ pulmonary imaging such as CT, MRI, or X-ray (iii) ex situ pulmonary imaging, such as from a biopsy, (iv) serum cytokine concentration determination, and (v) physical examination, such as by stethoscope. Patients administered the treatment show improved outcomes.

The patients are evaluated for overall survival through 28 days following treatment, and evaluated for secondary outcomes (i) length of hospital stay (ii) length of ICU stay, (iii) duration of ventilator use, (iv) duration of vasopressors use, (v) duration on renal replacement therapy, (vi) viral kinetics as measured by virologic failure (defined as increase in viral load of >0.5 log on two consecutive days, or >1 log increase in one day, not in keeping with any baseline trend of rising viral loads during the pre-treatment viral testing), and (vii) number of adverse events as measured by CTCAE v. 5.0, for at least six months following treatment. Patients administered the treatment show improved outcomes.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VIII. SEQUENCES

| SEQ ID | Sequence | Annotation |
|---|---|---|
| 1 | EPKSCDKTHTCPPCPAPELLGGPSVFLFPPKPKDTLMISR | WT IgG1 |
| | TPEVTCVVVDVSHEDPEVKFNWYVDGVEVHNAKTKPR | |
| | EEQYNSTYRVVSVLTVLHQDWLNGKEYKCKVSNKALP | |
| | APIEKTISKAKGQPREPQVYTLPPSREEMTKNQVSLTCLV | |
| | KGFYPSDIAVEWESNGQPENNYKTTPPVLDSDGSFFLYS | |
| | KLTVDKSRWQQGNVFSCSVMHEALHNHYTQKSLSLSP | |
| | GK | |
| 2 | TKVDKTVERKCCVECPPCPAPPVAGPSVFLFPPKPKDTL | WT IgG2 Fc |
| | MISRTPEVTCVVVDVSHEDPEVQFNWYVDGVEVHNAK | |
| | TKPREEQFNSTFRVVSVLTVVHQDWLNGKEYKCKVSN | |
| | KGLPAPIEKTISKTKGQPREPQVYTLPPSREEMTKNQVSL | |
| | TCLVKGFYPSDIAVEWESNGQPENNYKTTPPMLDSDGSF | |
| | FLYSKLTVDKSRWQQGNVFSCSVMHEALHNHYTQKSL | |
| | SLSPGK | |
| 3 | ESKYGPPCPSCPAPEFLGGPSVFLFPPKPKDTLMISRTPEV | WT IgG4 |
| | TCVVVDVSQEDPEVQFNWYVDGVEVHNAKTKPREEQF | |
| | NSTYRVVSVLTVLHQDWLNGKEYKCKVSNKGLPSSIEK | |
| | TISKAKGQPREPQVYTLPPSQEEMTKNQVSLTCLVKGFY | |
| | PSDIAVEWESNGQPENNYKTTPPVLDSDGSFFLYSRLTV | |
| | DKSRWQEGNVFSCSVMHEALHNHYTQKSLSLSLGK | |
| 4 | (GₘS)ₙ | GS Linker, wherein each |
| | | of m and n is an integer |
| | | between 1 to 4 |
| 5 | MGPAENKKVR FENTTSDKGK IPSKVIKSYY | NiVG protein attachment |
| | GTMDIKKINE GLLDSKILSA FNTVIALLGS IVIIVMNIMI | glycoprotein (602 aa) |
| | IQNYTRSTDN QAVIKDALQG IQQQIKGLAD | |
| | KIGTEIGPKV SLIDTSSTIT IPANIGLLGS KISQSTASIN | |
| | ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QC | |
| 6 | MMADSKLVSLNNNLSGKIKDOGKVIKNYYGTMDIKKIN | Hendra Virus G Protein |
| | DGLLDSKILGAFNTVIALLGSIIIIVMNIMIIQNYTRTTDNQ | |
| | ALIKESLQSVQQQIKALTDKIGTEIGPKVSLIDTSSTITIPA | |
| | NIGLLGSKISQSTSSINENVNDKCKFTLPPLKIHECNISCP | |
| | NPLPFREYRPISQGVSDLVGLPNQICLQKTTSTILKPRLIS | |
| | YTLPINTREGVCITDPLLAVDNGFFAYSHLEKIGSCTRGI | |
| | AKQRIIGVGEVLDRGDKVPSMFMTNVWTPPNPSTIHHCS | |
| | STYHEDFYYTLCAVSHVGDPILNSTSWTESLSLIRLAVRP | |
| | KSDSGDYNQKYIAITKVERGKYDKVMPYGPSGIKQGDT | |
| | LYFPAVGFLPRTEFQYNDSNCPIIHCKYSKAENCRLSMG | |
| | VNSKSHYILRSGLLKYNLSLGGDIILQFIEIADNRLTIGSPS | |
| | KIYNSLGQPVFYQASYSWDTMIKLGDVDTVDPLRVQW | |
| | RNNSVISRPGQSQCPRFNVCPEVCWEGTYNDAFLIDRLN | |
| | WVSAGVYLNSNQTAENPVFAVFKDNEILYQVPLAEDDT | |
| | NAQKTITDCFLLENVIWCISLVEIYDTGDSVIRPKLFAVKI | |
| | PAQCSES | |
| 7 | MADSKLVSLNNNLSGKIKDOGKVIKNYYGTMDIKKIND | Hendra Virus G Protein |
| | GLLDSKILGAFNTVIALLGSIIIIVMNIMIIQNYTRTTDNQA | without Met |
| | LIKESLQSVQQQIKALTDKIGTEIGPKVSLIDTSSTITIPANI | |
| | GLLGSKISQSTSSINENVNDKCKFTLPPLKIHECNISCPNP | |
| | LPFREYRPISQGVSDLVGLPNQICLQKTTSTILKPRLISYT | |
| | LPINTREGVCITDPLLAVDNGFFAYSHLEKIGSCTRGIAK | |
| | QRIIGVGEVLDRGDKVPSMFMTNVWTPPNPSTIHHCSST | |
| | YHEDFYYTLCAVSHVGDPILNSTSWTESLSLIRLAVRPKS | |
| | DSGDYNQKYIAITKVERGKYDKVMPYGPSGIKQGDTLY | |
| | FPAVGFLPRTEFQYNDSNCPIIHCKYSKAENCRLSMGVN | |
| | SKSHYILRSGLLKYNLSLGGDIILQFIEIADNRLTIGSPSKI | |
| | YNSLGQPVFYQASYSWDTMIKLGDVDTVDPLRVQWRN | |
| | NSVISRPGQSQCPRFNVCPEVCWEGTYNDAFLIDRLNW | |
| | VSAGVYLNSNQTAENPVFAVFKDNEILYQVPLAEDDTN | |
| | AQKTITDCFLLENVIWCISLVEIYDTGDSVIRPKLFAVKIP | |
| | AQCSES | |
| 8 | MPAENKKVRFENTTSDKGKIPSKVIKSYYGTMDIKKINE | Nipah Virus G Protein |
| | GLLDSKILSAFNTVIALLGSIVIIVMNIMIIQNYTRSTDNQ | |
| | AVIKDALQGIQQQIKGLADKIGTEIGPKVSLIDTSSTITIPA | |
| | NIGLLGSKISQSTASINENVNEKCKFTLPPLKIHECNISCP | |
| | NPLPFREYRPQTEGVSNLVGLPNNICLQKTSNQILKPKLI | |
| | SYTLPVVGQSGTCITDPLLAMDEGYFAYSHLERIGSCSR | |
| | GVSKQRIIGVGEVLDRGDEVPSLFMTNVWTPPNPNTVY | |
| | HCSAVYNNEFYYVLCAVSTVGDPILNSTYWSGSLMMTR | |
| | LAVKPKSNGGGYNQHQLALRSIEKGRYDKVMPYGPSGI | |
| | KQGDTLYFPAVGFLVRTEFKYNDSNCPITKCQYSKPENC | |
| | RLSMGIRPNSHYILRSGLLKYNLSDGENPKVVFIEISDQR | |
| | LSIGSPSKIYDSLGQPVFYQASFSWDTMIKFGDVLTVNPL | |
| | VVNWRNNTVISRPGQSQCPRFNTCPEICWEGVYNDAFLI | |
| | DRINWISAGVFLDSNQTAENPVFTVFKDNEILYRAQLAS | |
| | EDTNAQKTITNCFLLKNKIWCISLVEIYDTGDNVIRPKLF | |
| | AVKIPEQCT | |
| 9 | PAENKKVRFENTTSDKGKIPSKVIKSYYGTMDIKKINEG | Nipah Virus G Protein |
| | LLDSKILSAFNTVIALLGSIVIIVMNIMIIQNYTRSTDNQA | (No Met) |
| | VIKDALQGIQQQIKGLADKIGTEIGPKVSLIDTSSTITIPAN | |
| | IGLLGSKISQSTASINENVNEKCKFTLPPLKIHECNISCPNP | |
| | LPFREYRPQTEGVSNLVGLPNNICLQKTSNQILKPKLISY | |
| | TLPVVGQSGTCITDPLLAMDEGYFAYSHLERIGSCSRGV | |
| | SKQRIIGVGEVLDRGDEVPSLFMTNVWTPPNPNTVYHCS | |
| | AVYNNEFYYVLCAVSTVGDPILNSTYWSGSLMMTRLA | |
| | VKPKSNGGGYNQHQLALRSIEKGRYDKVMPYGPSGIKQ | |
| | GDTLYFPAVGFLVRTEFKYNDSNCPITKCQYSKPENCRL | |
| | SMGIRPNSHYILRSGLLKYNLSDGENPKVVFIEISDQRLSI | |
| | GSPSKIYDSLGQPVFYQASFSWDTMIKFGDVLTVNPLVV | |
| | NWRNNTVISRPGQSQCPRFNTCPEICWEGVYNDAFLIDR | |
| | INWISAGVFLDSNQTAENPVFTVFKDNEILYRAQLASED | |
| | TNAQKTITNCFLLKNKIWCISLVEIYDTGDNVIRPKLFAV | |
| | KIPEQCT | |
| 10 | MLSQLQKNYLDNSNQQGDKMNNPDKKLSVNFNPLELD | Cedar Virus G Protein |
| | KGQKDLNKSYYVKNKNYNVSNLLNESLHDIKFCIYCIFS | |
| | LLIIITIINIITISIVITRLKVHEENNGMESPNLQSIQDSLSSL | |
| | TNMINTEITPRIGILVTATSVTLSSSINYVGTKTNQLVNEL | |
| | KDYITKSCGFKVPELKLHECNISCADPKISKSAMYSTNA | |
| | YAELAGPPKIFCKSVSKDPDFRLKQIDYVIPVQQDRSICM | |
| | NNPLLDISDGFFTYIHYEGINSCKKSDSFKVLLSHGEIVD | |
| | RGDYRPSLYLLSSHYHPYSMQVINCVPVTCNQSSFVFCH | |
| | ISNNTKTLDNSDYSSDEYYITYFNGIDRPKTKKIPINNMT | |
| | ADNRYIHFTFSGGGGVCLGEEFIIPVTTVINTDVFTHDYC | |
| | ESFNCSVQTGKSLKEICSESLRSPTNSSRYNLNGIMIISQN | |
| | NMTDFKIQLNGITYNKLSFGSPGRLSKTLGQVLYYQSSM | |
| | SWDTYLKAGFVEKWKPFTPNWMNNTVISRPNQGNCPR | |
| | YHKCPEICYGGTYNDIAPLDLGKDMYVSVILDSDQLAE | |
| | NPEITVFNSTTILYKERVSKDELNTRSTTTSCFLFLDEPW | |
| | CISVLETNRFNGKSIRPEIYSYKIPKYC | |
| 11 | LSQLQKNYLDNSNQQGDKMNNPDKKLSVNFNPLELDK | Cedar Virus G Protein |
| | GQKDLNKSYYVKNKNYNVSNLLNESLHDIKFCIYCIFSL | (No Met) |
| | LIIITIINIITISIVITRLKVHEENNGMESPNLQSIQDSLSSLT | |
| | NMINTEITPRIGILVTATSVTLSSSINYVGTKTNQLVNELK | |
| | DYITKSCGFKVPELKLHECNISCADPKISKSAMYSTNAY | |
| | AELAGPPKIFCKSVSKDPDFRLKQIDYVIPVQQDRSICMN | |
| | NPLLDISDGFFTYIHYEGINSCKKSDSFKVLLSHGEIVDR | |
| | GDYRPSLYLLSSHYHPYSMQVINCVPVTCNQSSFVFCHI | |
| | SNNTKTLDNSDYSSDEYYITYFNGIDRPKTKKIPINNMTA | |
| | DNRYIHFTFSGGGGVCLGEEFIIPVTTVINTDVFTHDYCE | |
| | SFNCSVQTGKSLKEICSESLRSPTNSSRYNLNGIMIISQNN | |
| | MTDFKIQLNGITYNKLSFGSPGRLSKTLGQVLYYQSSMS | |
| | WDTYLKAGFVEKWKPFTPNWMNNTVISRPNQGNCPRY | |
| | HKCPEICYGGTYNDIAPLDLGKDMYVSVILDSDQLAENP | |
| | EITVFNSTTILYKERVSKDELNTRSTTTSCFLFLDEPWCIS | |
| | VLETNRFNGKSIRPEIYSYKIPKYC | |
| 12 | MPQKTVEFINMNSPLERGVSTLSDKKTLNQSKITKQGYF | Bat Paramyxovirus G |
| | GLGSHSERNWKKQKNQNDHYMTVSTMILEILVVLGIMF | Protein |
| | NLIVLTMVYYQNDNINQRMAELTSNITVLNLNLNQLTN | |
| | KIQREIIPRITLIDTATTITIPSAITYILATLTTRISELLPSINQ | |
| | KCEFKTPTLVLNDCRINCTPPLNPSDGVKMSSLATNLVA | |
| | HGPSPCRNFSSVPTIYYYRIPGLYNRTALDERCILNPRLTI | |
| | SSTKFAYVHSEYDKNCTRGFKYYELMTFGEILEGPEKEP | |
| | RMFSRSFYSPTNAVNYHSCTPIVTVNEGYFLCLECTSSDP | |
| | LYKANLSNSTFHLVILRHNKDEKIVSMPSFNLSTDQEYV | |
| | QIIPAEGGGTAESGNLYFPCIGRLLHKRVTHPLCKKSNCS | |
| | RTDDESCLKSYYNQGSPQHQVVNCLIRIRNAQRDNPTW | |
| | DVITVDLTNTYPGSRSRIFGSFSKPMLYQSSVSWHTLLQ | |
| | VAEITDLDKYQLDWLDTPYISRPGGSECPFGNYCPTVCW | |
| | EGTYNDVYSLTPNNDLFVTVYLKSEQVAENPYFAIFSRD | |
| | QILKEFPLDAWISSARTTTISCFMFNNEIWCIAALEITRLN | |
| | DDIIRPIYYSFWLPTDCRTPYPHTGKMTRVPLRSTYNY | |
| 13 | PQKTVEFINMNSPLERGVSTLSDKKTLNQSKITKQGYFG | Bat Paramyxovirus G |
| | LGSHSERNWKKQKNQNDHYMTVSTMILEILVVLGIMFN | Protein (No Met) |
| | LIVLTMVYYQNDNINQRMAELTSNITVLNLNLNQLTNKI | |
| | QREIIPRITLIDTATTITIPSAITYILATLTTRISELLPSINQKC | |
| | EFKTPTLVLNDCRINCTPPLNPSDGVKMSSLATNLVAHG | |
| | PSPCRNFSSVPTIYYYRIPGLYNRTALDERCILNPRLTISST | |
| | KFAYVHSEYDKNCTRGFKYYELMTFGEILEGPEKEPRM | |
| | FSRSFYSPTNAVNYHSCTPIVTVNEGYFLCLECTSSDPLY | |
| | KANLSNSTFHLVILRHNKDEKIVSMPSFNLSTDQEYVQII | |
| | PAEGGGTAESGNLYFPCIGRLLHKRVTHPLCKKSNCSRT | |
| | DDESCLKSYYNQGSPQHQVVNCLIRIRNAQRDNPTWDV | |
| | ITVDLTNTYPGSRSRIFGSFSKPMLYQSSVSWHTLLQVAE | |
| | ITDLDKYQLDWLDTPYISRPGGSECPFGNYCPTVCWEGT | |
| | YNDVYSLTPNNDLFVTVYLKSEQVAENPYFAIFSRDQIL | |
| | KEFPLDAWISSARTTTISCFMFNNEIWCIAALEITRLNDDI | |
| | IRPIYYSFWLPTDCRTPYPHTGKMTRVPLRSTYNY | |
| 14 | MATNRDNTITSAEVSQEDKVKKYYGVETAEKVADSISG | Mojiang virus, Tongguan |
| | NKVFILMNTLLILTGAIITITLNITNLTAAKSQQNMLKIIQ | 1 G Protein |
| | DDVNAKLEMFVNLDQLVKGEIKPKVSLINTAVSVSIPGQ | |
| | ISNLQTKFLQKYVYLEESITKQCTCNPLSGIFPTSGPTYPP | |
| | TDKPDDDTTDDDKVDTTIKPIEYPKPDGCNRTGDHFTM | |
| | EPGANFYTVPNLGPASSNSDECYTNPSFSIGSSIYMFSQEI | |
| | RKTDCTAGEILSIQIVLGRIVDKGQQGPQASPLLVWAVP | |
| | NPKIINSCAVAAGDEMGWVLCSVTLTAASGEPIPHMFD | |
| | GFWLYKLEPDTEVVSYRITGYAYLLDKQYDSVFIGKGG | |
| | GIQKGNDLYFQMYGLSRNRQSFKALCEHGSCLGTGGGG | |
| | YQVLCDRAVMSFGSEESLITNAYLKVNDLASGKPVIIGQ | |
| | TFPPSDSYKGSNGRMYTIGDKYGLYLAPSSWNRYLRFGI | |
| | TPDISVRSTTWLKSQDPIMKILSTCTNTDRDMCPEICNTR | |
| | GYQDIFPLSEDSEYYTYIGITPNNGGTKNFVAVRDSDGHI | |
| | ASIDILQNYYSITSATISCFMYKDEIWCIAITEGKKQKDNP | |
| | QRIYAHSYKIRQMCYNMKSATVTVGNAKNITIRRY | |
| 15 | ATNRDNTITSAEVSQEDKVKKYYGVETAEKVADSISGN | Mojiang virus, Tongguan |
| | KVFILMNTLLILTGAIITITLNITNLTAAKSQQNMLKIIQD | 1 G (No Met) |
| | DVNAKLEMFVNLDQLVKGEIKPKVSLINTAVSVSIPGQIS | |
| | NLQTKFLQKYVYLEESITKQCTCNPLSGIFPTSGPTYPPT | |
| | DKPDDDTTDDDKVDTTIKPIEYPKPDGCNRTGDHFTMEP | |
| | GANFYTVPNLGPASSNSDECYTNPSFSIGSSIYMFSQEIR | |
| | KTDCTAGEILSIQIVLGRIVDKGQQGPQASPLLVWAVPN | |
| | PKIINSCAVAAGDEMGWVLCSVTLTAASGEPIPHMFDGF | |
| | WLYKLEPDTEVVSYRITGYAYLLDKQYDSVFIGKGGGIQ | |
| | KGNDLYFQMYGLSRNRQSFKALCEHGSCLGTGGGGYQ | |
| | VLCDRAVMSFGSEESLITNAYLKVNDLASGKPVIIGQTFP | |
| | PSDSYKGSNGRMYTIGDKYGLYLAPSSWNRYLRFGITPD | |
| | ISVRSTTWLKSQDPIMKILSTCTNTDRDMCPEICNTRGYQ | |
| | DIFPLSEDSEYYTYIGITPNNGGTKNFVAVRDSDGHIASI | |
| | DILQNYYSITSATISCFMYKDEIWCIAITEGKKQKDNPQR | |
| | IYAHSYKIRQMCYNMKSATVTVGNAKNITIRRY | |
| 16 | MATQEVRLKCLLCGIIVLVLSLEGLGILHYEKLSKIGLVK | Hendra virus F Protein |
| | GITRKYKIKSNPLTKDIVIKMIPNVSNVSKCTGTVMENY | |
| | KSRLTGILSPIKGAIELYNNNTHDLVGDVKLAGVVMAGI | |
| | AIGIATAAQITAGVALYEAMKNADNINKLKSSIESTNEA | |
| | VVKLQETAEKTVYVLTALQDYINTNLVPTIDQISCKQTE | |
| | LALDLALSKYLSDLLFVFGPNLQDPVSNSMTIQAISQAF | |
| | GGNYETLLRTLGYATEDFDDLLESDSIAGQIVYVDLSSY | |
| | YIIVRVYFPILTEIQQAYVQELLPVSFNNDNSEWISIVPNF | |
| | VLIRNTLISNIEVKYCLITKKSVICNQDYATPMTASVREC | |
| | LTGSTDKCPRELVVSSHVPRFALSGGVLFANCISVTCQC | |
| | QTTGRAISQSGEQTLLMIDNTTCTTVVLGNIIISLGKYLGS | |
| | INYNSESIAVGPPVYTDKVDISSQISSMNQSLQQSKDYIK | |
| | EAQKILDTVNPSLISMLSMIILYVLSIAALCIGLITFISFVIV | |
| | EKKRGNYSRLDDRQVRPVSNGDLYYIGT | |
| 17 | ILHYEKLSKIGLVKGITRKYKIKSNPLTKDIVIKMIPNVSN | Hendra virus F Protein, |
| | VSKCTGTVMENYKSRLTGILSPIKGAIELYNNNTHDLVG | Without signal sequence |
| | DVKLAGVVMAGIAIGIATAAQITAGVALYEAMKNADNI | |
| | NKLKSSIESTNEAVVKLQETAEKTVYVLTALQDYINTNL | |
| | VPTIDQISCKQTELALDLALSKYLSDLLFVFGPNLQDPVS | |
| | NSMTIQAISQAFGGNYETLLRTLGYATEDFDDLLESDSIA | |
| | GQIVYVDLSSYYIIVRVYFPILTEIQQAYVQELLPVSFNN | |
| | DNSEWISIVPNFVLIRNTLISNIEVKYCLITKKSVICNQDY | |
| | ATPMTASVRECLTGSTDKCPRELVVSSHVPRFALSGGVL | |
| | FANCISVTCQCQTTGRAISQSGEQTLLMIDNTTCTTVVL | |
| | GNIIISLGKYLGSINYNSESIAVGPPVYTDKVDISSQISSM | |
| | NQSLQQSKDYIKEAQKILDTVNPSLISMLSMIILYVLSIAA | |
| | LCIGLITFISFVIVEKKRGNYSRLDDRQVRPVSNGDLYYI | |
| | GT | |
| 18 | MVVILDKRCYCNLLILILMISECSVGILHYEKLSKIGLVK | Nipah virus F Protein |
| | GVTRKYKIKSNPLTKDIVIKMIPNVSNMSQCTGSVMENY | |
| | KTRLNGILTPIKGALEIYKNNTHDLVGDVRLAGVIMAGV | |
| | AIGIATAAQITAGVALYEAMKNADNINKLKSSIESTNEA | |
| | VVKLQETAEKTVYVLTALQDYINTNLVPTIDKISCKQTE | |
| | LSLDLALSKYLSDLLFVFGPNLQDPVSNSMTIQAISQAFG | |
| | GNYETLLRTLGYATEDFDDLLESDSITGQIIYVDLSSYYII | |
| | VRVYFPILTEIQQAYIQELLPVSFNNDNSEWISIVPNFILV | |
| | RNTLISNIEIGFCLITKRSVICNQDYATPMTNNMRECLTG | |
| | STEKCPRELVVSSHVPRFALSNGVLFANCISVTCQCQTT | |
| | GRAISQSGEQTLLMIDNTTCPTAVLGNVIISLGKYLGSVN | |
| | YNSEGIAIGPPVFTDKVDISSQISSMNQSLQQSKDYIKEA | |
| | QRLLDTVNPSLISMLSMIILYVLSIASLCIGLITFISFIIVEK | |
| | KRNTYSRLEDRRVRPTSSGDLYYIGT | |
| 19 | ILHYEKLSKIGLVKGVTRKYKIKSNPLTKDIVIKMIPNVS | Nipah virus F Protein, |
| | NMSQCTGSVMENYKTRLNGILTPIKGALEIYKNNTHDL | without signal sequence |
| | VGDVRLAGVIMAGVAIGIATAAQITAGVALYEAMKNAD | |
| | NINKLKSSIESTNEAVVKLQETAEKTVYVLTALQDYINT | |
| | NLVPTIDKISCKQTELSLDLALSKYLSDLLFVFGPNLQDP | |
| | VSNSMTIQAISQAFGGNYETLLRTLGYATEDFDDLLESD | |
| | SITGQIIYVDLSSYYIIVRVYFPILTEIQQAYIQELLPVSFN | |
| | NDNSEWISIVPNFILVRNTLISNIEIGFCLITKRSVICNQDY | |
| | ATPMTNNMRECLTGSTEKCPRELVVSSHVPRFALSNGV | |
| | LFANCISVTCQCQTTGRAISQSGEQTLLMIDNTTCPTAVL | |
| | GNVIISLGKYLGSVNYNSEGIAIGPPVFTDKVDISSQISSM | |
| | NQSLQQSKDYIKEAQRLLDTVNPSLISMLSMIILYVLSIA | |
| | SLCIGLITFISFIIVEKKRNTYSRLEDRRVRPTSSGDLYYIG | |
| | T | |
| 20 | MSNKRTTVLIIISYTLFYLNNAAIVGFDFDKLNKIGVVQG | Cedar Virus F Protein |
| | RVLNYKIKGDPMTKDLVLKFIPNIVNITECVREPLSRYNE | |
| | TVRRLLLPIHNMLGLYLNNTNAKMTGLMIAGVIMGGIAI | |
| | GIATAAQITAGFALYEAKKNTENIQKLTDSIMKTQDSIDK | |
| | LTDSVGTSILILNKLQTYINNQLVPNLELLSCRQNKIEFDL | |
| | MLTKYLVDLMTVIGPNINNPVNKDMTIQSLSLLFDGNY | |
| | DIMMSELGYTPQDFLDLIESKSITGQIIYVDMENLYVVIR | |
| | TYLPTLIEVPDAQIYEFNKITMSSNGGEYLSTIPNFILIRGN | |
| | YMSNIDVATCYMTKASVICNQDYSLPMSQNLRSCYQGE | |
| | TEYCPVEAVIASHSPRFALTNGVIFANCINTICRCQDNGK | |
| | TITQNINQFVSMIDNSTCNDVMVDKFTIKVGKYMGRKDI | |
| | NNINIQIGPQIIIDKVDLSNEINKMNQSLKDSIFYLREAKRI | |
| | LDSVNISLISPSVQLFLIIISVLSFIILLIIIVYLYCKSKHSYK | |
| | YNKFIDDPDYYNDYKRERINGKASKSNNIYYVGD | |
| 21 | TVLIIISYTLFYLNNAAIVGFDFDKLNKIGVVQGRVLNYK | Cedar Virus F Protein, |
| | IKGDPMTKDLVLKFIPNIVNITECVREPLSRYNETVRRLL | without signal sequence |
| | LPIHNMLGLYLNNTNAKMTGLMIAGVIMGGIAIGIATAA | |
| | QITAGFALYEAKKNTENIQKLTDSIMKTQDSIDKLTDSV | |
| | GTSILILNKLQTYINNQLVPNLELLSCRQNKIEFDLMLTK | |
| | YLVDLMTVIGPNINNPVNKDMTIQSLSLLFDGNYDIMMS | |
| | ELGYTPQDFLDLIESKSITGQIIYVDMENLYVVIRTYLPTL | |
| | IEVPDAQIYEFNKITMSSNGGEYLSTIPNFILIRGNYMSNI | |
| | DVATCYMTKASVICNQDYSLPMSQNLRSCYQGETEYCP | |
| | VEAVIASHSPRFALTNGVIFANCINTICRCQDNGKTITQNI | |
| | NQFVSMIDNSTCNDVMVDKFTIKVGKYMGRKDINNINI | |
| | QIGPQIIIDKVDLSNEINKMNQSLKDSIFYLREAKRILDSV | |
| | NISLISPSVQLFLIIISVLSFIILLIIIVYLYCKSKHSYKYNKFI | |
| | DDPDYYNDYKRERINGKASKSNNIYYVGD | |
| 22 | MALNKNMFSSLFLGYLLVYATTVQSSIHYDSLSKVGVIK | Mojiang virus, Tongguan |
| | GLTYNYKIKGSPSTKLMVVKLIPNIDSVKNCTQKQYDEY | 1 F Protein |
| | KNLVRKALEPVKMAIDTMLNNVKSGNNKYRFAGAIMA | |
| | GVALGVATAATVTAGIALHRSNENAQAIANMKSAIQNT | |
| | NEAVKQLQLANKQTLAVIDTIRGEINNNIIPVINQLSCDTI | |
| | GLSVGIRLTQYYSEIITAFGPALQNPVNTRITIQAISSVFN | |
| | GNFDELLKIMGYTSGDLYEILHSELIRGNIIDVDVDAGYI | |
| | ALEIEFPNLTLVPNAVVQELMPISYNIDGDEWVTLVPRF | |
| | VLTRTTLLSNIDTSRCTITDSSVICDNDYALPMSHELIGCL | |
| | QGDTSKCAREKVVSSYVPKFALSDGLVYANCLNTICRC | |
| | MDTDTPISQSLGATVSLLDNKRCSVYQVGDVLISVGSYL | |
| | GDGEYNADNVELGPPIVIDKIDIGNQLAGINQTLQEAED | |
| | YIEKSEEFLKGVNPSIITLGSMVVLYIFMILIAIVSVIALVL | |
| | SIKLTVKGNVVRQQFTYTQHVPSMENINYVSH | |
| 23 | IHYDSLSKVGVIKGLTYNYKIKGSPSTKLMVVKLIPNIDS | Mojiang virus, Tongguan |
| | VKNCTQKQYDEYKNLVRKALEPVKMAIDTMLNNVKSG | 1 F Protein, without |
| | NNKYRFAGAIMAGVALGVATAATVTAGIALHRSNENA | signal sequence |
| | QAIANMKSAIQNTNEAVKQLQLANKQTLAVIDTIRGEIN | |
| | NNIIPVINQLSCDTIGLSVGIRLTQYYSEIITAFGPALQNPV | |
| | NTRITIQAISSVFNGNFDELLKIMGYTSGDLYEILHSELIR | |
| | GNIIDVDVDAGYIALEIEFPNLTLVPNAVVQELMPISYNI | |
| | DGDEWVTLVPRFVLTRTTLLSNIDTSRCTITDSSVICDND | |
| | YALPMSHELIGCLQGDTSKCAREKVVSSYVPKFALSDGL | |
| | VYANCLNTICRCMDTDTPISQSLGATVSLLDNKRCSVYQ | |
| | VGDVLISVGSYLGDGEYNADNVELGPPIVIDKIDIGNQLA | |
| | GINQTLQEAEDYIEKSEEFLKGVNPSIITLGSMVVLYIFMI | |
| | LIAIVSVIALVLSIKLTVKGNVVRQQFTYTQHVPSMENIN | |
| | YVSH | |
| 24 | MKKKTDNPTISKRGHNHSRGIKSRALLRETDNYSNGLIV | Bat Paramyxovirus F |
| | ENLVRNCHHPSKNNLNYTKTQKRDSTIPYRVEERKGHY | Protein |
| | PKIKHLIDKSYKHIKRGKRRNGHNGNIITIILLLILILKTQM | |
| | SEGAIHYETLSKIGLIKGITREYKVKGTPSSKDIVIKLIPNV | |
| | TGLNKCTNISMENYKEQLDKILIPINNIIELYANSTKSAPG | |
| | NARFAGVIIAGVALGVAAAAQITAGIALHEARQNAERIN | |
| | LLKDSISATNNAVAELQEATGGIVNVITGMQDYINTNLV | |
| | PQIDKLQCSQIKTALDISLSQYYSEILTVFGPNLQNPVTTS | |
| | MSIQAISQSFGGNIDLLLNLLGYTANDLLDLLESKSITGQI | |
| | TYINLEHYFMVIRVYYPIMTTISNAYVQELIKISFNVDGS | |
| | EWVSLVPSYILIRNSYLSNIDISECLITKNSVICRHDFAMP | |
| | MSYTLKECLTGDTEKCPREAVVTSYVPRFAISGGVIYAN | |
| | CLSTTCQCYQTGKVIAQDGSQTLMMIDNQTCSIVRIEEIL | |
| | ISTGKYLGSQEYNTMHVSVGNPVFTDKLDITSQISNINQS | |
| | IEQSKFYLDKSKAILDKINLNLIGSVPISILFIIAILSLILSIIT | |
| | FVIVMIIVRRYNKYTPLINSDPSSRRSTIQDVYIIPNPGEHS | |
| | IRSAARSIDRDRD | |
| 25 | SRALLRETDNYSNGLIVENLVRNCHHPSKNNLNYTKTQ | Bat Paramyxovirus F |
| | KRDSTIPYRVEERKGHYPKIKHLIDKSYKHIKRGKRRNG | Protein, without signal |
| | HNGNIITIILLLILILKTQMSEGAIHYETLSKIGLIKGITREY | sequence |
| | KVKGTPSSKDIVIKLIPNVTGLNKCTNISMENYKEQLDKI | |
| | LIPINNIIELYANSTKSAPGNARFAGVIIAGVALGVAAAA | |
| | QITAGIALHEARQNAERINLLKDSISATNNAVAELQEAT | |
| | GGIVNVITGMQDYINTNLVPQIDKLQCSQIKTALDISLSQ | |
| | YYSEILTVFGPNLQNPVTTSMSIQAISQSFGGNIDLLLNLL | |
| | GYTANDLLDLLESKSITGQITYINLEHYFMVIRVYYPIMT | |
| | TISNAYVQELIKISFNVDGSEWVSLVPSYILIRNSYLSNIDI | |
| | SECLITKNSVICRHDFAMPMSYTLKECLTGDTEKCPREA | |
| | VVTSYVPRFAISGGVIYANCLSTTCQCYQTGKVIAQDGS | |
| | QTLMMIDNQTCSIVRIEEILISTGKYLGSQEYNTMHVSVG | |
| | NPVFTDKLDITSQISNINQSIEQSKFYLDKSKAILDKINLN | |
| | LIGSVPISILFIIAILSLILSIITFVIVMIIVRRYNKYTPLINSD | |
| | PSSRRSTIQDVYIIPNPGEHSIRSAARSIDRDRD | |
| 26 | MVVILDKRCY CNLLILILMI SECSVG | signal sequence |
| 27 | ILHYEKLSKIGLVKGVTRKYKIKSNPLTKDIVIKMIPNVS | Nipah virus NiV-F F2 (aa |
| | NMSQCTGSVMENYKTRLNGILTPIKGALEIYKNNTHDL | 27-109) |
| | VGDVR | |
| 28 | LAGVIMAGVAIGIATAAQITAGVALYEAMKNADNINKL | Nipah virus NiV F F1 (aa |
| | KSSIESTNEAVVKLQETAEKTVYVLTALQDYINTNLVPTI | 110-546) |
| | DKISCKQTELSLDLALSKYLSDLLFVFGPNLQDPVSNSM | |
| | TIQAISQAFGGNYETLLRTLGYATEDFDDLLESDSITGQII | |
| | YVDLSSYYIIVRVYFPILTEIQQAYIQELLPVSFNNDNSE | |
| | WISIVPNFILVRNTLISNIEIGFCLITKRSVICNQDYATPMT | |
| | NNMRECLTGSTEKCPRELVVSSHVPRFALSNGVLFANCI | |
| | SVTCQCQTTGRAISQSGEQTLLMIDNTTCPTAVLGNVIIS | |
| | LGKYLGSVNYNSEGIAIGPPVFTDKVDISSQISSMNQSLQ | |
| | QSKDYIKEAQRLLDTVNPSLISMLSMIILYVLSIASLCIGLI | |
| | TFISFIIVEKKRNTYSRLEDRRVRPTSSGDLYYIGT | |
| 29 | MKVR FENTTSDKGK IPSKVIKSYY GTMDIKKINE | NiVG protein attachment |
| | GLLDSKILSA FNTVIALLGS IVIIVMNIMI IQNYTRSTDN | glycoprotein |
| | QAVIKDALQG IQQQIKGLAD KIGTEIGPKV SLIDTSSTIT | Truncated Δ5 |
| | IPANIGLLGS KISQSTASIN ENVNEKCKFT LPPLKIHECN | |
| | ISCPNPLPFR EYRPQTEGVS NLVGLPNNIC | |
| | LQKTSNQILK PKLISYTLPV VGQSGTCITD | |
| | PLLAMDEGYF AYSHLERIGS CSRGVSKQRI | |
| | IGVGEVLDRG DEVPSLFMTN VWTPPNPNTV | |
| | YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF | |
| | LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PEICWEGVYN DAFLIDRINW ISAGVFLDSN | |
| | QTAENPVFTV FKDNEILYRA QLASEDTNAQ | |
| | KTITNCFLLK NKIWCISLVE IYDTGDNVIR PKLFAVKIPE | |
| | QCT | |
| 30 | MSKVIKSYY GTMDIKKINE GLLDSKILSA FNTVIALLGS | NiVG protein attachment |
| | IVIIVMNIMI IQNYTRSTDN QAVIKDALQG | glycoprotein |
| | IQQQIKGLAD KIGTEIGPKV SLIDTSSTIT IPANIGLLGS | Truncated Δ20 |
| | KISQSTASIN ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QCT | |
| 31 | MSYY GTMDIKKINE GLLDSKILSA FNTVIALLGS | NiVG protein attachment |
| | IVIIVMNIMI IQNYTRSTDN QAVIKDALQG | glycoprotein |
| | IQQQIKGLAD KIGTEIGPKV SLIDTSSTIT IPANIGLLGS | Truncated Δ25 |
| | KISQSTASIN ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QCT | |
| 32 | ILHY EKLSKIGLVK GVTRKYKIKS NPLTKDIVIK | Nipah virus NiV-F F0 |
| | MIPNVSNMSQ CTGSVMENYK TRLNGILTPI | T234 truncation (aa 525- |
| | KGALEIYKNQ THDLVGDVRL AGVIMAGVAI | 544) AND mutation on |
| | GIATAAQITA GVALYEAMKN ADNINKLKSS | N-linked glycosylation |
| | IESTNEAVVK LQETAEKTVY VLTALQDYIN | site |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 33 | MVVILDKRCY CNLLILILMI SECSVGILHY | Truncated NiV fusion |
| | EKLSKIGLVK GVTRKYKIKS NPLTKDIVIK | glycoprotein (FcDelta22) |
| | MIPNVSNMSQ CTGSVMENYK TRLNGILTPI | at cytoplasmic tail |
| | KGALEIYKNN THDLVGDVRL AGVIMAGVAI | (with signal sequence) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 34 | MKKINEGLLDSKILSA FNTVIALLGS IVIIVMNIMI | NiVG protein attachment |
| | IQNYTRSTDN QAVIKDALQG IQQQIKGLAD | glycoprotein |
| | KIGTEIGPKV SLIDTSSTIT IPANIGLLGS KISQSTASIN | Truncated and mutated |
| | ENVNEKCKFT LPPLKIHECN ISCPNPLPFR | (E501 A, W504A, |
| | EYRPQTEGVS NLVGLPNNIC LQKTSNQILK | Q530A, E533A) NiV G |
| | PKLISYTLPV VGQSGTCITD PLLAMDEGYF | protein (Gc Δ 34) |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PAICAEGVYN | |
| | DAFLIDRINW ISAGVFLDSN ATAANPVFTV | |
| | FKDNEILYRA QLASEDTNAQ KTITNCFLLK | |
| | NKIWCISLVE IYDTGDNVIR PKLFAVKIPE QCT | |
| 35 | KKINEGLLDSKILSA FNTVIALLGS IVIIVMNIMI | NiVG protein attachment |
| | IQNYTRSTDN QAVIKDALQG IQQQIKGLAD | glycoprotein |
| | KIGTEIGPKV SLIDTSSTIT IPANIGLLGS KISQSTASIN | Truncated and mutated |
| | ENVNEKCKFT LPPLKIHECN ISCPNPLPFR | (E501 A, W504A, |
| | EYRPQTEGVS NLVGLPNNIC LQKTSNQILK | Q530A, E533A) NiV G |
| | PKLISYTLPV VGQSGTCITD PLLAMDEGYF | protein (Gc Δ 34) |
| | AYSHLERIGS CSRGVSKQRI IGVGEVLDRG | Without N-terminal |
| | DEVPSLFMTN VWTPPNPNTV YHCSAVYNNE | methionine |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PAICAEGVYN | |
| | DAFLIDRINW ISAGVFLDSN ATAANPVFTV | |
| | FKDNEILYRA QLASEDTNAQ KTITNCFLLK | |
| | NKIWCISLVE IYDTGDNVIR PKLFAVKIPE QCT | |
| 36 | MVVILDKRCY CNLLILILMI SECSVGILHY | Truncated NiV fusion |
| | EKLSKIGLVK GVTRKYKIKS NPLTKDIVIK | glycoprotein (FcDelta22) |
| | MIPNVSNMSQ CTGSVMENYK TRLNGILTPI | at cytoplasmic tail |
| | KGALEIYKNN THDLVGDVRL AGVIMAGVAI | (with signal sequence) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 37 | ILHY EKLSKIGLVK GVTRKYKIKS NPLTKDIVIK | Nipah virus NiV-F F0 |
| | MIPNVSNMSQ CTGSVMENYK TRLNGILTPI | T234 truncation (aa 525- |
| | KGALEIYKNN THDLVGDVRL AGVIMAGVAI | 544) |
| | GIATAAQITA GVALYEAMKN ADNINKLKSS | |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNTGT | |
| 38 | ILHY EKLSKIGLVK GVTRKYKIKS NPLTKDIVIK | Truncated mature NiV |
| | MIPNVSNMSQ CTGSVMENYK TRLNGILTPI | fusion glycoprotein |
| | KGALEIYKNN THDLVGDVRL AGVIMAGVAI | (FcDelta22) at |
| | GIATAAQITA GVALYEAMKN ADNINKLKSS | cytoplasmic tail |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN | |
| | NMRECLTGST EKCPRELVVS SHVPRFALSN | |
| | GVLFANCISV TCQCQTTGRA ISQSGEQTLL | |
| | MIDNTTCPTA VLGNVIISLG KYLGSVNYNS | |
| | EGIAIGPPVF TDKVDISSQI SSMNQSLQQS | |
| | KDYIKEAQRL LDTVNPSLIS MLSMIILYVL SIASLCIGLI | |
| | TFISFIIVEK KRNT | |
| 39 | FNTVIALLGS IVIIVMNIMI IQNYTRSTDN | NivG protein attachment |
| | QAVIKDALQG IQQQIKGLAD KIGTEIGPKV SLIDTSSTIT | glycoprotein |
| | IPANIGLLGS KISQSTASIN ENVNEKCKFT LPPLKIHECN | Without cytoplasmic tail |
| | ISCPNPLPFR EYRPQTEGVS NLVGLPNNIC | Uniprot Q9IH62 |
| | LQKTSNQILK PKLISYTLPV VGQSGTCITD | |
| | PLLAMDEGYF AYSHLERIGS CSRGVSKQRI | |
| | IGVGEVLDRG DEVPSLFMTN VWTPPNPNTV | |
| | YHCSAVYNNE FYYVLCAVST VGDPILNSTY | |
| | WSGSLMMTRL AVKPKSNGGG YNQHQLALRS | |
| | IEKGRYDKVM PYGPSGIKQG DTLYFPAVGF | |
| | LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PEICWEGVYN DAFLIDRINW ISAGVFLDSN | |
| | QTAENPVFTV FKDNEILYRA QLASEDTNAQ | |
| | KTITNCFLLK NKIWCISLVE IYDTGDNVIR PKLFAVKIPE | |
| | QC | |
| 40 | MMADSKLVSL NNNLSGKIKD QGKVIKNYYG | Hendra virus G protein |
| | TMDIKKINDG LLDSKILGAF | Uniprot O89343 |
| | NTVIALLGSI IIIVMNIMII QNYTRTTDNQ ALIKESLQSV | |
| | QQQIKALTDK IGTEIGPKVS LIDTSSTITI PANIGLLGSK | |
| | ISQSTSSINE NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD | |
| | KVPSMFMTNV WTPPNPSTIH HCSSTYHEDF | |
| | YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD | |
| | TVDPLRVQWR NNSVISRPGQ SQCPRFNVCP | |
| | EVCWEGTYND AFLIDRLNWV SAGVYLNSNQ | |
| | TAENPVFAVF KDNEILYQVP LAEDDTNAQK | |
| | TITDCFLLEN VIWCISLVEI YDTGDSVIRP KLFAVKIPAQ | |
| | CSES | |
| 41 | MADSKLVSL NNNLSGKIKD QGKVIKNYYG | Hendra virus G protein |
| | TMDIKKINDG LLDSKILGAF | Uniprot O89343 Without |
| | NTVIALLGSI IIIVMNIMII QNYTRTTDNQ ALIKESLQSV | N-terminal methionine |
| | QQQIKALTDK IGTEIGPKVS LIDTSSTITI PANIGLLGSK | |
| | ISQSTSSINE NVNDKCKFTL | |
| | PPLKIHECNI SCPNPLPFRE YRPISQGVSD LVGLPNQICL | |
| | QKTTSTILKP RLISYTLPIN TREGVCITDP LLAVDNGFFA | |
| | YSHLEKIGSC TRGIAKQRII GVGEVLDRGD | |
| | KVPSMFMTNV WTPPNPSTIH HCSSTYHEDF | |
| | YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ PVFYQASYSW DTMIKLGDVD | |
| | TVDPLRVQWR NNSVISRPGQ SQCPRFNVCP | |
| | EVCWEGTYND AFLIDRLNWV SAGVYLNSNQ | |
| | TAENPVFAVF KDNEILYQVP LAEDDTNAQK | |
| | TITDCFLLEN VIWCISLVEI YDTGDSVIRP KLFAVKIPAQ | |
| | CSES | |
| 42 | FNTVIALLGSI IIIVMNIMII QNYTRTTDNQ ALIKESLQSV | SARS CoV 2 S(0) |
| | QQQIKALTDK | |
| | IGTEIGPKVS LIDTSSTITI PANIGLLGSK ISQSTSSINE | |
| | NVNDKCKFTL | |
| | PPLKIHECNI SCPNPLPFRE YRPISQGVSD LVGLPNQICL | |
| | QKTTSTILKP | |
| | RLISYTLPIN TREGVCITDP LLAVDNGFFA YSHLEKIGSC | |
| | TRGIAKQRII | |
| | GVGEVLDRGD KVPSMFMTNV WTPPNPSTIH | |
| | HCSSTYHEDF YYTLCAVSHV | |
| | GDPILNSTSW TESLSLIRLA VRPKSDSGDY | |
| | NQKYIAITKV ERGKYDKVMP | |
| | YGPSGIKQGD TLYFPAVGFL PRTEFQYNDS | |
| | NCPIIHCKYS KAENCRLSMG | |
| | VNSKSHYILR SGLLKYNLSL GGDIILQFIE IADNRLTIGS | |
| | PSKIYNSLGQ | |
| | PVFYQASYSW DTMIKLGDVD TVDPLRVQWR | |
| | NNSVISRPGQ SQCPRFNVCP EVCWEGTYND | |
| | AFLIDRLNWV SAGVYLNSNQ TAENPVFAVF | |
| | KDNEILYQVP LAEDDTNAQK TITDCFLLEN | |
| | VIWCISLVEI YDTGDSVIRP KLFAVKIPAQ CSES | |
| 43 | MFVFLVLLPLVSSQCVNLTTRTQLPPAYTNSFTRGVYYPDKVF | SARS CoV 2 S(0) |
| | RSSVLHSTQDLFLPFFSNVTWFHAIHVSGTNGTKRFDNPVLPF | Uniprot P0DTC2 |
| | NDGVYFASTEKSNIIRGWIFGTTLDSKTQSLLIVNNATNVVIKV | with signal |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWTA | |
| | GAAAYYVGYLQPRTFLLKYNENGTITDAVDCALDPLSETKCT | |
| | LKSFTVEKGIYQTSNFRVQPTESIVRFPNITNLCPFGEVFNATRF | |
| | ASVYAWNRKRISNCVADYSVLYNSASFSTFKCYGVSPTKLND | |
| | LCFTNVYADSFVIRGDEVRQIAPGQTGKIADYNYKLPDDFTGC | |
| | VIAWNSNNLDSKVGGNYNYLYRLFRKSNLKPFERDISTEIYQA | |
| | GSTPCNGVEGFNCYFPLQSYGFQPTNGVGYQPYRVVVLSFEL | |
| | LHAPATVCGPKKSTNLVKNKCVNFNFNGLTGTGVLTESNKKF | |
| | LPFQQFGRDIADTTDAVRDPQTLEILDITPCSFGGVSVITPGTNT | |
| | SNQVAVLYQDVNCTEVPVAIHADQLTPTWRVYSTGSNVFQTR | |
| | AGCLIGAEHVNNSYECDIPIGAGICASYQTQTNSPRRARSVASQ | |
| | SIIAYTMSLGAENSVAYSNNSIAIPTNFTISVTTEILPVSMTKTSV | |
| | DCTMYICGDSTECSNLLLQYGSFCTQLNRALTGIAVEQDKNTQ | |
| | EVFAQVKQIYKTPPIKDFGGFNFSQILPDPSKPSKRSFIEDLLFN | |
| | KVTLADAGFIKQYGDCLGDIAARDLICAQKFNGLTVLPPLLTD | |
| | EMIAQYTSALLAGTITSGWTFGAGAALQIPFAMQMAYRFNGI | |
| | GVTQNVLYENQKLIANQFNSAIGKIQDSLSSTASALGKLQDVV | |
| | NQNAQALNTLVKQLSSNFGAISSVLNDILSRLDKVEAEVQIDR | |
| | LITGRLQSLQTYVTQQLIRAAEIRASANLAATKMSECVLGQSK | |
| | RVDFCGKGYHLMSFPQSAPHGVVFLHVTYVPAQEKNFTTAPA | |
| | ICHDGKAHFPREGVFVSNGTHWFVTQRNFYEPQIITTDNTFVS | |
| | GNCDVVIGIVNNTVYDPLQPELDSFKEELDKYFKNHTSPDVDL | |
| | GDISGINASVVNIQKEIDRLNEVAKNLNESLIDLQELGKYEQYI | |
| | KWPWYIWLGFIAGLIAIVMVTIMLCCMTSCCSCLKGCCSCGSC | |
| | CKFDEDDSEPVLKGVKLHYT | |
| 44 | MGPAENKKVR FENTTSDKGK IPSKVIKSYY | NiVG protein attachment |
| | GTMDIKKINE GLLDSKILSA FNTVIALLGS IVIIVMNIMI | glycoprotein (602 aa) |
| | IQNYTRSTDN QAVIKDALQG IQQQIKGLAD | |
| | KIGTEIGPKV SLIDTSSTIT IPANIGLLGS KISQSTASIN | |
| | ENVNEKCKFT LPPLKIHECN ISCPNPLPFR | |
| | EYRPQTEGVS NLVGLPNNIC LQKTSNQILK | |
| | PKLISYTLPV VGQSGTCITD PLLAMDEGYF | |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG SPSKIYDSLG | |
| | QPVFYQASFS WDTMIKFGDV LTVNPLVVNW | |
| | RNNTVISRPG QSQCPRFNTC PEICWEGVYN | |
| | DAFLIDRINW ISAGVFLDSN QTAENPVFTV | |
| | FKDNEILYRA QLASEDTNAQ KTITNCFLLK | |
| | NKIWCISLVE IYDTGDNVIR PKLFAVKIPE QC | |
| 45 | SQCVNLTTRTQLPPAYTNSFTRGVYYPDKVFRSSVLHST | SARS COV 2 S(0) |
| | QDLFLPFFSNVTWFHAIHVSGTNGTKRFDNPVLPFNDGV | Uniprot P0DTC2 |
| | YFASTEKSNIIRGWIFGTTLDSKTQSLLIVNNATNVVIKV | No signal |
| | CEFQFCNDPFLGVYYHKNNKSWMESEFRVYSSANNCTF | |
| | EYVSQPFLMDLEGKQGNFKNLREFVFKNIDGYFKIYSKH | |
| | TPINLVRDLPQGFSALEPLVDLPIGINITRFQTLLALHRSY | |
| | LTPGDSSSGWTAGAAAYYVGYLQPRTFLLKYNENGTIT | |
| | DAVDCALDPLSETKCTLKSFTVEKGIYQTSNFRVQPTESI | |
| | VRFPNITNLCPFGEVFNATRFASVYAWNRKRISNCVADY | |
| | SVLYNSASFSTFKCYGVSPTKLNDLCFTNVYADSFVIRG | |
| | DEVRQIAPGQTGKIADYNYKLPDDFTGCVIAWNSNNLD | |
| | SKVGGNYNYLYRLFRKSNLKPFERDISTEIYQAGSTPCN | |
| | GVEGFNCYFPLQSYGFQPTNGVGYQPYRVVVLSFELLH | |
| | APATVCGPKKSTNLVKNKCVNFNFNGLTGTGVLTESNK | |
| | KFLPFQQFGRDIADTTDAVRDPQTLEILDITPCSFGGVSVI | |
| | TPGTNTSNQVAVLYQDVNCTEVPVAIHADQLTPTWRVY | |
| | STGSNVFQTRAGCLIGAEHVNNSYECDIPIGAGICASYQT | |
| | QTNSPRRARSVASQSIIAYTMSLGAENSVAYSNNSIAIPT | |
| | NFTISVTTEILPVSMTKTSVDCTMYICGDSTECSNLLLQY | |
| | GSFCTQLNRALTGIAVEQDKNTQEVFAQVKQIYKTPPIK | |
| | DFGGFNFSQILPDPSKPSKRSFIEDLLFNKVTLADAGFIKQ | |
| | YGDCLGDIAARDLICAQKFNGLTVLPPLLTDEMIAQYTS | |
| | ALLAGTITSGWTFGAGAALQIPFAMQMAYRFNGIGVTQ | |
| | NVLYENQKLIANQFNSAIGKIQDSLSSTASALGKLQDVV | |
| | NQNAQALNTLVKQLSSNFGAISSVLNDILSRLDKVEAEV | |
| | QIDRLITGRLQSLQTYVTQQLIRAAEIRASANLAATKMSE | |
| | CVLGQSKRVDFCGKGYHLMSFPQSAPHGVVFLHVTYVP | |
| | AQEKNFTTAPAICHDGKAHFPREGVFVSNGTHWFVTQR | |
| | NFYEPQIITTDNTFVSGNCDVVIGIVNNTVYDPLQPELDS | |
| | FKEELDKYFKNHTSPDVDLGDISGINASVVNIQKEIDRLN | |
| | EVAKNLNESLIDLQELGKYEQYIKWPWYIWLGFIAGLIAI | |
| | VMVTIMLCCMTSCCSCLKGCCSCGSCCKFDEDDSEPVL | |
| | KGVKLHYT | |
| 46 | QIPRDRLSNIGVIVDEGKSLKIAGSHESRYIVLSLVP | Sendai F protein |
| | GVDFENGCGTAQVIQYKSLLNRLLIPLRDALDLQEA | |
| | LITVINDTTQNAGAPQSRFFGAVIGTIALGVATSAQI | |
| | TAGIALAEAREAKRDIALIKESMTKTHKSIELLQNA | |
| | VGEQILALKTLQDFVNDEIKPAISELGCETAALRLGI | |
| | KLTQHYSELLTAFGSNFGTIGEKSLTLQALSSLYSA | |
| | NITEIMTTIKTGQSNIYDVIYTEQIKGTVIDVDLERY | |
| | MVTLSVKIPILSEVPGVLIHKASSISYNIDGEEWYVT | |
| | VPSHILSRASFLGGADITDCVESRLTYICPRDPAQLIP | |
| | DSQQKCILGDTTRCPVTKVVDSLIPKFAFVNGGVV | |
| | ANCIASTCTCGTGRRPISQDRSKGVVFLTHDNCGLI | |
| | GVNGVELYANRRGHDATWGVQNLTVGPAIAIRPID | |
| | ISLNLADATNFLQDSKAELEKARKILSEVGRWYNSR | |
| | ETVITIIVVMVVILVVIIVIIIVLYRLRRSMLMGNPDD | |
| | RIPRDTYTLEPKIRHMYTNGGFDAMAEKR | |
| 47 | MDGDRGKRDSYWSTSPSGSTTKPASGWERSSKADTWL | Sendai HN protein |
| | LILSFTQWALSIATVIICIIISARQGYSMKEYSMTVEALNM | |
| | SSREVKESLTSLIRQEVIARAVNIQSSVQTGIPVLLNKNSR | |
| | DVIQMIDKSCSRQELTQHCESTIAVHHADGIAPLEPHSF | |
| | WRCPVGEPYLSSDPEISLLPGPSLLSGSTTISGCVRLPSLSI | |
| | GEAIYAYSSNLITQGCADIGKSYQVLQLGYISLNSDMFP | |
| | DLNPVVSHTYDINDNRKSCSVVATGTRGYQLCSMPTVD | |
| | ERTDYSSDGIEDLVLDVLDLKGRTKSHRYRNSEVDLDH | |
| | PFSALYPSVGNGIATEGSLIFLGYGGLTTPLQGDTKCRTQ | |
| | GCQQVSQDTCNEALKITWLGGKQVVSVIIQVNDYLSER | |
| | PKIRVTTIPITQNYLGAEGRLLKLGDRVYIYTRSSGWHSQ | |
| | LQIGVLDVSHPLTINWTPHEALSRPGNKECNWYNKCPK | |
| | ECISGVYTDAYPLSPDAANVATVTLYANTSRVNPTIMYS | |
| | NTTNIINMLRIKDVQLEAAYTTTSCITHFGKGYCFHIIEIN | |
| | QKSLNTLQPMLFKTSIPKLCKAES | |
| 48 | MWSELKIRSNDGGEGPEDANDPRGKGVQHIHIQPSLPVG | Sendai HN protein |
| | QRVRMDGDRGKRDSYWSTSPSGSTTKPASGWERSSKA | (modified CTD) |
| | DTWLLILSFTQWALSIATVIICIIISARQGYSMKEYSMTVE | |
| | ALNMSSREVKESLTSLIRQEVIARAVNIQSSVQTGIPVLL | |
| | NKNSRDVIQMIDKSCSRQELTQHCESTIAVHHADGIAPL | |
| | EPHSFWRCPVGEPYLSSDPEISLLPGPSLLSGSTTISGCVR | |
| | LPSLSIGEAIYAYSSNLITQGCADIGKSYQVLQLGYISLNS | |
| | DMFPDLNPVVSHTYDINDNRKSCSVVATGTRGYQLCSM | |
| | PTVDERTDYSSDGIEDLVLDVLDLKGRTKSHRYRNSEV | |
| | DLDHPFSALYPSVGNGIATEGSLIFLGYGGLTTPLQGDTK | |
| | CRTQGCQQVSQDTCNEALKITWLGGKQVVSVIIQVNDY | |
| | LSERPKIRVTTIPITQNYLGAEGRLLKLGDRVYIYTRSSG | |
| | WHSQLQIGVLDVSHPLTINWTPHEALSRPGNKECNWYN | |
| | KCPKECISGVYTDAYPLSPDAANVATVTLYANTSRVNPT | |
| | IMYSNTTNIINMLRIKDVQLEAAYTTTSCITHFGKGYCFH | |
| | IIEINQKSLNTLQPMLFKTSIPKLCKAES | |
| 49 | DIQMTQSPSSLSASVGDRVTITCQASQDITNYLNWYQQK | REGN10933 Fab light |
| | PGKAPKLLIYAASNLETGVPSRFSGSGSGTDFTFTISGLQ | chain |
| | PEDIATYYCQQYDNLPLTFGGGTKVEIKRTVAAPSVFIFP | |
| | PSDEQLKSGTASVVCLLNNFYPREAKVQWKVDNALQS | |
| | GNSQESVTEQDSKDSTYSLSSTLTLSKADYEKHKVYACE | |
| | VTHQGLSSPVTKSFNRGEC | |
| 50 | QVQLVESGGGLVKPGGSLRLSCAASGFTFSDYYMSWIR | REGN10933 Fab heavy |
| | QAPGKGLEWVSYITYSGSTIYYADSVKGRFTISRDNAKS | chain |
| | SLYLQMNSLRAEDTAVYYCARDRGTTMVPFDYWGQGT | |
| | LVTVSSASTKGPSVFPLAPSSKSTSGGTAALGCLVKDYF | |
| | PEPVTVSWNSGALTSGVHTFPAVLQSSGLYSLSSVVTVP | |
| | SSSLGTQTYICNVNHKPSNTKVDKKVEPKSCDKT | |
| 51 | QSALTQPASVSGSPGQSITISCTGTSSDVGGYNYVSWYQ | REGN10987 Fab light |
| | QHPGKAPKLMIYDVSKRPSGVSNRFSGSKSGNTASLTIS | chain |
| | GLQSEDEADYYCNSLTSISTWVFGGGTKLTVLGQPKAA | |
| | PSVTLFPPSSEELQANKATLVCLISDFYPGAVTVAWKAD | |
| | SSPVKAGVETTTPSKQSNNKYAASSYLSLTPEQWKSHRS | |
| | YSCQVTHEGSTVEKTVAPTECS | |
| 52 | QVQLVESGGGVVQPGRSLRLSCAASGFTFSNYAMYWV | REGN10987 Fab heavy |
| | RQAPGKGLEWVAVISYDGSNKYYADSVKGRFTISRDNS | chain |
| | KNTLYLQMNSLRTEDTAVYYCASGSDYGDYLLVYWGQ | |
| | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDK | |
| 53 | DIVMTQSPSSLSASVGDRVTITCRASQSISRYLNWYQQK | JS016 light chain |
| | PGKAPKLLIYAASSLQSGVPSRFSGSGSGTDFTLTISSLQP | |
| | EDFATYYCQQSYSTPPEYTFGQGTKLEIKRTVAAPSVFIF | |
| | PPSDEQLKSGTASVVCLLNNFYPREAKVQWKVDNALQS | |
| | GNSQESVTEQDSKDSTYSLSSTLTLSKADYEKHKVYACE | |
| | VTHQGLSSPVTKSFNRGECS | |
| 54 | EVQLVESGGGLVQPGGSLRLSCAASGFTVSSNYMSWVR | JS016 heavy chain |
| | QAPGKGLEWVSVIYSGGSTFYADSVKGRFTISRDNSMN | |
| | TLFLQMNSLRAEDTAVYYCARVLPMYGDYLDYWGQG | |
| | TLVTVSSASTKGPSVFPLAPSSKSTSGGTAALGCLVKDY | |
| | FPEPVTVSWNSGALTSGVHTFPAVLQSSGLYSLSSVVTV | |
| | PSSSLGTQTYICNVNHKPSNTKVDKRVEPKSCDKTHTHH | |
| | HHHH | |
| 55 | QVQLQQSGAELVRPGVSVKISCKGSGYTFTDYAMHWV | 15G11 VDJ |
| | KQSHAKSLEWIGVISTYYGDASYNQKFKGKATMTVDKS | |
| | SSTAYMELARLTSEDSAIYYCARWANWGYYYAMDYW | |
| | GQGTSVTVSS | |
| 56 | QAVVTQESALTTSPGETVTLTCRSSTGAVTTSNYANWV | 15G11 VJ |
| | QEKPDHLFTGLIGGTNNRAPGVPARFSGSLIGDKAALTIT | |
| | GAQTEDEAIYFCALWYSNHWVFGGGTKLTVL | |
| 57 | QVQLKESGPGLVAPSQSLSITCTVSGFSLTSYAISWVRQP | 18F4 VDJ |
| | PGKGLEWLGVIWTGGGTNYNSALKSRLSISKDNSKSQV | |
| | FLKMNSLQTDDTARYYCARKDYYGSSRNAMDYWGQG | |
| | TSVTVSS | |
| 58 | QAVVTQESALTTSPGETVTLTCRSSTGAVTTSNYANWV | 18F4 VJ |
| | QEKPDHLFTGLIGGTNNRAPGVPARFSGSLIGDKAALTIT | |
| | GAQTEDEAIYFCALWYSNHWVFGGGTKLTVL | |
| 59 | QVQLQQPGAELVKPGASVKMSCKASGYTFTSYWITWV | 1E5 VDJ |
| | KQRPGQGLEWIGDIYPGSGSTNYNEKFKSKATLTVDTSS | |
| | STAYMQLSSLTSEDSAVYYCARSTVVATDAMDYWGQG | |
| | TSVTVSS | |
| 60 | DIVMTQAAFSNPVTLGTSASISCRSSKSLLHSNGITYLYW | 1E5 VJ |
| | YLQKPGQSPQLLIYQMSNLASGVPDRFSSSGSGTDFTLRI | |
| | SRVEAEDVGVYYCAQNLELPWTFGGGTKLEIK | |
| 61 | QVQLKESGPGLVAPSQSLSITCTVSGFSLTSYAISWVRQP | 1G3 VDJ |
| | PGKGLEWLGVIWTGGGTNYNSALKSRLSISKDNSKSQV | |
| | FLKMNSLQTDDTARYYCARFHYYGSSYGYFDYWGQGT | |
| | TLTVSS | |
| 62 | QAVVTQESALTTSPGETVTLTCRSSTGAVTTSNYANWV | 1G3 VJ |
| | QEKPDHLFTGLIGGTNNRAPGVPARFSGSLIGDKAALTIT | |
| | GAQTEDEAIYFCALWYSNHWVFGGGTKLTVL | |
| 63 | QVQLKESGPGLVAPSQSLSITCTVSGFSLTSYAISWVRQP | 21C3 VDJ |
| | PGKGLEWLGVIWTGGGTNYNSALKSRLSISKDNSKSQV | |
| | FLKMNSLQTDDTARYYCARIYYYGSSYFDYWGQGTTLT | |
| | VSS | |
| 64 | QAVVTQESALTTSPGETVTLTCRSSTGAVTTSNYANWV | 21C3 VJ |
| | QEKPDHLFTGLIGGTNNRAPGVPARFSGSLIGDKAALTIT | |
| | GAQTEDEAIYFCALWYSNQFIFGSGTKVTVL | |
| 65 | QVQLQQSGAELVRPGTSVKVSCKASGYAFTNYLIEWVK | 22D9 VDJ |
| | QRPGQGLEWIGVINPGSGGTNYNEKFKGKATLTADKSS | |
| | STAYMQLSSLTSEDSAVYFCARRHYYYGVDYWGQGTT | |
| | LTVSS | |
| 66 | DIKMTQSPSSMYASLGERVTITCKASQDINSYLSWFQQK | 22D9 VJ |
| | PGKSPKTLIYRANRLVDGVPSRFSGSGSGQDYSLTISSLE | |
| | YEDMGIYYCLQYDELYTFGGGTKLEIK | |
| 67 | EVQLQQSGPELVKPGASVKISCKTSGYTFTEYTMYWVK | 23D11 VDJ |
| | QSHGKSLEWIGGVNPNNGDTSYSQKFKGKATLTVDKSS | |
| | STAYMELRSLTSEDSAVYYCARDGYDLYYGMDYWGQ | |
| | GTSVTVSS | |
| 68 | DIVMTQSQKFMSTSVGDRVSVTCKASQNVGTNVAWYQ | 23D11 VJ |
| | QKPGQSPKALIYSASYRYSGVPDRFTGSGSGTDFTLTISN | |
| | VQSVDLAEYFCHQYNSYPWTFGGGTKLEIR | |
| 69 | EVQLQQSGAELVRPGASVKLSCTASGFNIKDDYMHWV | 26E2 VDJ |
| | KQRPEQGLEWIGWIDPENGDTEYASKFQGKATITADTSS | |
| | NTAYLQLSSLTSEDTAVYYCTKGYYGSSYDYFDYWGQ | |
| | GTTLTVSS | |
| 70 | DIQMTQSPSSLSASLGGKVTITCKASQDINKYIAWYQHK | 26E2 VJ |
| | PGKGPRLLIHYTSTLQPGIPSRFSGSGSGRDYSFSISNLEP | |
| | EDIATYYCLQYDNLWTFGGGTKLEIK | |
| 71 | QVQLQQSGPELVKPGASVKISCKASGYAFSSSWMNWV | 29F7 VDJ |
| | KQRPGKGLEWIGRIYPGDGDTNYNGKFKGKATLTADKS | |
| | SSTAYMQLSSLTSEDSAVYFCARDDYDEGDYWGQGTTL | |
| | TVSS | |
| 72 | DIQMTQSPSSLSASLGGKVTITCKASQDINKYIAWYQHK | 29F7 VJ |
| | PGKGPRLLIHYTSTLQPGIPSRFSGSGSGRDYSFSISNLEP | |
| | EDIATYYCLQYDNLWTFGGGTKLEIK | |
| 73 | DVKLVESGEGLVKPGGSLKLSCAASGFTFSSYAMSWVR | 3B3 VDJ |
| | QTPEKRLEWVAYISSGGDYIYYADTVKGRFTISRDNARN | |
| | TLYLQMSSLKSEDTAMYYCTRVARLYDGYFDYWGQGT | |
| | TLTVSS | |
| 74 | NIVLTQSPASLAVSLGQRATISCRASESVDSYGNSFMHW | 3B3 VJ |
| | YQQKPGQPPKLLIYLASNLESGVPARFSGSGSRTDFTLTI | |
| | DPVEADDAATYYCQQNNEDPYTFGGGTKLEIK | |
| 75 | EVQLQQSGAELVRPGASVKLSCTASGFNIKDDYMHWV | 3F2 VDJ |
| | KQRPEQGLEWIGWIDPENGDTEYASKFQGKATITVDISS | |
| | NTAYLQLNSLTSEDTAVYYCSTLIYYYGSSNDYWGQGT | |
| | TLTVSS | |
| 76 | QAVVTQESALTTSPGETVTLTCRSSTGAVTTSNYANWV | 3F2 VJ |
| | QEKPDHLFTGLIGGTNNRAPGVPARFSGSLIGDKAALTIT | |
| | GAQTEDEAIYFCALWYSNHWVFGGGTKLTVL | |
| 77 | QVQLKESGPGLVAPSQSLSITCTVSGFSLTSYAISWVRQP | D59047-11955 VDJ |
| | PGKGLEWLGVIWTGGGTNYNSALKSRLSISKDNSKSQV | |
| | FLKMNSLQTDDTARYYCARIYYYGSSYFDYWGQGTTLT | |
| | VSS | |
| 78 | QAVVTQESALTTSPGETVTLTCRSSTGAVTTSNYANWV | D59047-11955 VJ |
| | QEKPDHLFTGLIGGTNNRAPGVPARFSGSLIGDKAALTIT | |
| | GAQTEDEAIYFCALWYSNQFIFGSGTKVTVL | |
| 79 | EVQLVESGGGLVKPGGSLRLSCATSGFTFSHYSMNWVR | D70678-12637-S1 VDJ |
| | QAPGKGLEWVSSISSSSSNIYYADSVKGRFTVSRDNAKN | |
| | SLYLQMNSLRAEDTAVYYCARRGSSWSFDYWGQGTLV | |
| | TVSS | |
| 80 | EIVLTQSPATLSLSPGERAILSCRASQSISSTYLAWNQQKP | D70678-12637-S1 VJ |
| | GQAPRLLIYGASSRATGIPDRFSGSGSGTDFTLTISRLEPE | |
| | DFAVYYCQQYGSSWTFGQGTKVEIK | |
| 81 | QVQLVQSGAEVKKPGASVKVSCKASGYTFTDYYMHW | D70678-12799-S1 VDJ |
| | VRQAPGQGLEWMGWINPNSGGTNYAQKFQGRVTMTR | |
| | DTSISTAYMELSRLRSDDTALYYCARLDYWSQGTLVTV | |
| | SS | |
| 82 | DIVMTQTPLSLPVTLGQPASISCRSSQSLVHSDGNTYLSW | D70678-12799-S1 VJ |
| | LQQRPGQPPRLLIYKISNRFSGVPDRFSGSGAGTDFTLKIS | |
| | RVEAEDVGVYYCMQETQFTWTFGQGTKVEIK | |
| 83 | QVQLQESGPGLVKPSETLSLTCTVSGGSISSYYWTWIRQ | D70678-13531-S1 VDJ |
| | SPGKGLEWIGYIYYSGSTYTNPSLKSRVTFSVDTSENQFS | |
| | LKLNSVTAADTAIYFCARDNMDVWGKGTTVTVSS | |
| 84 | DVVMTQTPLSLPVTLGQPASISCRCSQSLVYSDGNTYLN | D70678-13531-S1 VJ |
| | WFQQRPGQSPRRLIYKVSIRDSGVPDRFSGSGSGTDFTL | |
| | KISRVEAEDVGIYYCMQGTHRPITFGRGTRLEIK | |
| 85 | QVQLQESGPGLVKPSETLSLTCTVSGGSISNYYWTWIRQ | D70678-13576-S1 VDJ |
| | PPGKGLEWIGYIYYSGSTYTNPSLKSRVTISVDTSENQFS | |
| | LKLNSVTAADTAIYYCARDNMDVWGKGTTVTVSS | |
| 86 | DIVMTQSPDSLAVSLGERATINCKSSQSVLYSSNNKNYL | D70678-13576-S1 VJ |
| | AWYQQKPGQPPKLLIYWASTRESGVPDRFSGSGSGTDF | |
| | TLTISSLQAEDVAVYYCQQYYSTPYTFGQGTKLEIK | |
| 87 | QVQLVQSGAEVKKPGASVKVSCKASGYTFSSYGIIWVR | D70678-14004-S2 VDJ |
| | QAPGQGLEWMGWISAYNGNTNYAQKLQGRVTMTTDT | |
| | STSTAYMELRTLRSDDTAVYYCAREITLNWNYAGWFDP | |
| | WGQGTLVTVSS | |
| 88 | EIVMTQSPATLSVSPGERATLSCRASQSIRNNLAWYQQK | D70678-14004-S2 VJ |
| | PGQAPRLLIYGAISRATGVPARFSGSGSGTEFTLTISSLQS | |
| | EDFAVYHCQQYNNWLPYTFGQGTKLEIK | |
| 89 | EVQLVESGGGLVKPGGSLRLSCAASGFTFSYAWMTWV | D70678-14027-S2 VDJ |
| | RQAPGKGLEWVGRIKTKSDGGTTDYASPVKGKFTISRD | |
| | DSKNTLYLQMNSLQTEDTAVYYCTTHSSPDYWGQGTL | |
| | VTVSS | |
| 90 | DIQMTQSPSSLSASVGDRVTITCQASQDIRNYLNWYHQK | D70678-14027-S2 VJ |
| | PGKAPKLLIYDASNLETGVPSRFSGSGSGTDFTFTISSLQP | |
| | EDIATYYCQQYDNLPYTFGQGTKLEIK | |
| 91 | QVQLVESGGDLVKPGGSLRLSCAASGFTFSNAWMTWV | D70678-2155-S1 VDJ |
| | RQAPGKELEWVGRIKTKSDGGTIEYGVSVKGRFTISRDD | |
| | SKNTLFLQMNSLTTEDTAVYYCTTHSSPDYWGQGTLVT | |
| | VSS | |
| 92 | DIQMTQSPSSLSASVGDRVTITCQASQDIRNYLNWYQQK | D70678-2155-S1 VJ |
| | PGKAPKLLIYDASTLETGVPSRFSGSGSGTDFTFTISSLQP | |
| | EDIATYYCHQYGNLPLSFGGGTKVEIK | |
| 93 | QVQLQESGPGLVKPSETLSLTCTVSGGSISSYYWTWIRQ | D70678-2743-S1 VDJ |
| | PPGKGLEWIGYIYSSGSTYTNPSLKSRVTISVDTSENQFS | |
| | LKLNSVTAADTAVYYCARDNMDVWGKGTTVTVSS | |
| 94 | DVVMTQTPLSLPVTLGQPASISCRSSQSLVYSDGNTYLN | D70678-2743-S1 VJ |
| | WFQQRPGQSPRRLIYKVSNRDSGVPDRFSGSGSGTDFTL | |
| | KISRVEAEDVGIYYCMQGTHWPIIFGRGTRLEIK | |
| 95 | EVQLVESGGGLVKPGGSLRLSCAASGFTFNYAWMTWV | D70678-5521-S2 VDJ |
| | RQAPGKGLDWVGRIKTKTDSGTTDYAAPVKGRFTISRD | |
| | DSKNTLYLQMNNLKTEDTAVYYCTTHSTPDYWGQGTL | |
| | VTVSS | |
| 96 | DIQMTQSPSSLSASVGDRVTITCQASQDINNHLNWYQQK | D70678-5521-S2 VJ |
| | PGKAPNLLIYDASNLETGVPSRFSGSGSGTDFTFTISSLQP | |
| | EDFATYYCQQYDNLPYTFGQGTKLEIK | |

1. A binding agent, comprising: (i) at least one binding domain that binds to a viral protein exposed on the surface of a virus, and (ii) a modified Fe domain, wherein the modified Fe domain comprises one or more amino acid substitutions compared to wildtype Fe domain, wherein the binding agent is capable of neutralizing the virus and exhibits reduced pro-inflammatory activity compared to an unmodified Fc domain.

2. A binding agent, comprising: (i) at least one binding domain that binds to a viral protein exposed on the surface of a virus, and (ii) an Fc domain with reduced pro-inflammatory activity as compared to a wild-type IgG1 Fc domain, wherein the binding agent is capable of neutralizing the virus.

3. The binding agent of claim 2, wherein the Fc domain is an IgG2 or IgG4 Fc domain.

4. The binding agent of claim 1 or claim 2, wherein the Fc domain is a modified Fc domain that is modified by one or more amino acid substitutions compared to a wildtype IgG1 Fc domain.

5. The binding agent of claim 1 or claim 4, wherein the modified Fc domain exhibits reduced binding to an Fc activating receptor.

6. The binding agent of claim 1 or claim 4, wherein the modified Fc domain exhibits increased binding to an Fc inhibitory receptor compared to a wild-type Fc domain.

7. A binding agent, comprising (i) at least one binding domain that binds to a surface exposed viral protein, and (ii) a modified Fc domain, wherein the modified Fc domain comprises one or more amino acid substitutions compared to wildtype Fc domain, and wherein the modified Fc domain has decreased binding to at least one Fc activating receptor family member compared to the wild-type Fc domain.

8. The binding agent of claim 5 or claim 7, wherein the Fc activating receptor is Fe gamma receptor I (FcγRI), Fc gamma receptor IIA (FcγRIIA) or Fc gamma receptor III (FcγRIII).

9. The binding agent of any of claims 1-5, 7 and 8, wherein the binding agent is capable of forming an immune complex with decreased pro-inflammatory activity compared to an immune complex formed with a binding agent comprising the at least one binding domain and a wild-type Fc domain.

10. The binding agent of any of claims 1-9, wherein the wildtype Fc domain is a wildtype IgG1.

11. The binding agent of any of claims 1, 4, 5 and 7-10, wherein the modified Fc domain comprises an amino acid substitution selected from, Ser228Pro, Glu233Pro, Leu234Ala, Leu234Glu, Leu235Ala, Leu235Glu, Leu235Phe, Gly236Arg, Gly237Ala, Pro238Ser, Asp265Ala, His268Ala, His268Gln, Ser288Pro, Asn297Ala, Asn297Gly, Asn297Gln, Val309Leu, Gly318Ala, Leu328Arg, Pro329Gly, Ala330Ser, and Pro331Ser, each based on EU numbering, or combinations of any of the foregoing.

12. The binding agent of any of claims 1-4, 5, 7-9 and 11, wherein:
the modified Fc domain comprises a Leu235Glu substitution based on EU numbering;
the modified Fc domain comprises a Leu234Ala substitution based on EU numbering and Leu235Ala substitution based on EU numbering;
the modified Fc domain comprises a Ser288Pro substitution based on EU numbering and Leu235Glu substitution based on EU numbering;
the modified Fc domain comprises a Leu234Ala substitution based on EU numbering, Leu235Ala substitution based on EU numbering, and Pro329Gly substitution based on EU numbering;
the modified Fc domain comprises a Pro331Ser substitution based on EU numbering, Leu234Glu substitution based on EU numbering, and Leu235Phe substitution based on EU numbering;
the modified Fc domain comprises a Asp265Ala substitution based on EU numbering;
the modified Fe domain comprises a Gly237Ala substitution based on EU numbering;
the modified Fe domain comprises a Gly318Ala substitution based on EU numbering;
the modified Fe domain comprises a Glu233Pro substitution based on EU numbering;
the modified Fe domain comprises a Gly236Arg substitution based on EU numbering, Leu328Arg substitution based on EU numbering, and Pro329Gly substitution based on EU numbering;
the modified Fe domain comprises a His268Gln substitution based on EU numbering, Val309Leu substitution based on EU numbering, and Ala330Ser substitution based on EU numbering, and/or Pro331Ser substitution based on EU numbering;
the modified Fe domain comprises a Leu234Ala substitution based on EU numbering, Leu235Ala substitution based on EU numbering, Gly237Ala substitution based on EU numbering, Pro238Ser substitution based on EU numbering, His268Ala substitution based on EU numbering, Ala330Ser substitution based on EU numbering, and Pro331Ser substitution based on EU numbering;
the modified Fe domain comprises a Asn297Ala substitution based on EU numbering, Asn297Gly substitution based on EU numbering, or Asn297Gln substitution based on EU numbering; or
the modified Fe domain comprises a Ser228Pro substitution based on EU numbering, Phe234Ala substitution based on EU numbering, and Leu235Ala substitution based on EU numbering.

13. A binding agent, comprising (i) at least one binding domain that binds to a viral protein exposed on the surface of a virus, and (ii) a modified Fe domain, wherein the modified Fe domain comprises one or more amino acid substitutions compared to wildtype Fe domain, and wherein the modified Fe domain has increased binding to an inhibitory Fc receptor compared to the wild-type Fe domain.

14. The binding agent of claim 6 or 13, wherein the inhibitory Fc receptor is an FcγRIIB, optionally wherein the FcRIIB is FcγRIIB1 or FcγRIIB2.

15. The binding agent of any of claims 1, 2, 4, 6, 13 and 14, wherein:
the binding agent is capable of forming an immune complex with increased anti-inflammatory activity compared to an immune complex formed with a binding agent comprising the at least one binding domain and a wild-type Fc domain; or
the binding agent is capable of forming an immune complex with decreased inflammatory activity compared to an immune complex formed with a binding agent comprising the at least one binding domain and a wild-type Fc domain.

16. The binding agent of any of claims 1, 2, 4, 6, and 13-15, wherein the wildtype Fc domain is a wildtype IgG1.

17. The binding agent of any of claims 1, 4, 6, or 13-16, wherein the modified Fc domain comprises an amino acid substitution selected from, Phe241Ala, Ser267Glu, His268Phe, Leu328Phe, Ser324Thr, Pro238Asp, Leu328Glu, Ser239Asp, Ile332Glu, Gly236Ala each based on EU numbering, or combinations of any of the foregoing.

18. The binding agent of any of claims 1, 4, 6, or 13-17, wherein:
the modified Fc domain comprises a Ser267Glu substitution based on EU numbering and His268Phe substitution based on EU numbering, and Ser324Thr substitution based on EU numbering;
the modified Fc domain comprises a Ser267Glu substitution based on EU numbering and Leu328Phe substitution based on EU numbering;
the modified Fc domain comprises a Pro238Asp substitution based on EU numbering;
the modified Fc domain comprises a Leu328Glu substitution based on EU numbering;
the modified Fc domain comprises a Ser239Asp substitution based on EU numbering and Ile332Glu substitution based on EU numbering;
the modified Fc domain comprises a Ser239Asp substitution based on EU numbering and Ile332Glu substitution based on EU numbering, and Gly236Ala substitution based on EU numbering;
the modified Fc domain comprises a Ser267Glu substitution based on EU numbering;
the modified Fc domain comprises a E233D substitution based on EU numbering;
the modified Fc domain comprises a G237D substitution based on EU numbering;
the modified Fc domain comprises a H268D substitution based on EU numbering;
the modified Fe domain comprises a P271G substitution based on EU numbering;
the modified Fe domain comprises a A330R substitution based on EU numbering;
the modified Fe domain comprises a E233D substitution based on EU numbering and a A330R substitution based on EU numbering;
the modified Fe domain comprises a E233D substitution based on EU numbering, a P271G substitution based on EU numbering, and a A330R substitution based on EU numbering;
the modified Fe domain comprises a G237D substitution based on EU numbering, a H268D substitution based on EU numbering, and a P271G substitution based on EU numbering;
the modified Fe domain comprises a G237D substitution based on EU numbering, a P271G substitution based on EU numbering, and a A330R substitution based on EU numbering;
the modified Fe domain comprises a E233D substitution based on EU numbering, a H268D substitution based on EU numbering, a P271G substitution based on EU numbering, and a A330R substitution based on EU numbering;
the modified Fe domain comprises a G237D substitution based on EU numbering, a H268D substitution based on EU numbering, a P271G substitution based on EU numbering, and a A330R substitution based on EU numbering;
the modified Fe domain comprises a E233D substitution based on EU numbering, a G237D substitution based on EU numbering, a H268D substitution based on EU numbering, a P271G substitution based on EU numbering, and a A330R substitution based on EU numbering.

19. The binding agent of any of claims 1-18, wherein the at least one binding domain and modified Fe domain are indirectly linked via a linker, optionally wherein the linker is a peptide linker.

20. The binding agent of claim 19, wherein the peptide linker is (GₘS)ₙ(SEQ ID NO: 4), wherein each of m and n is an integer between 1 to 4, inclusive.

21. The binding agent of any of claims 1-20, wherein the at least one binding domain is at least two binding domains.

22. The binding agent of claim 21, wherein each of the at least two binding domains bind a distinct epitope of the viral protein.

23. The binding agent of any of claims 1-22, wherein the viral protein is a viral receptor.

24. The binding agent of any of claims 1-23, wherein the virus is an RNA virus.

25. The binding agent of any of claims 1-24, wherein the virus is a coronavirus.

26. The binding agent of claim 25, wherein the virus is Severe Acute Respiratory Syndrome (SARS) CoV-2.

27. The binding agent of claim 25, wherein the virus is SARS CoV-1.

28. The binding agent of claim 25, wherein the virus is Middle Eastern Respiratory Syndrome Virus (MERS-V).

29. The binding agent of any of claims 1-24, wherein the virus is an orthomyxovirus, optionally wherein the virus is an influenza virus.

30. The binding agent of any of claims 1-24, wherein the virus is a paramyxovirus.

31. The binding agent of claim 30, wherein the virus is a Respiratory Syncytial Virus (RSV) or is a Measles morbillivirus (MeV).

32. The binding agent of any of claims 1-31, wherein the binding agent is capable of neutralizing the virus and/or the binding agent reduces or prevents viral attachment to a host cell.

33. The binding agent of any of claims 1-28 and 32 wherein the virus is a coronavirus and the at least one binding domain specifically binds the S (spike) glycoprotein of the coronavirus.

34. The binding agent of any of claims 1-33, wherein the at least one binding domain comprises an antigen-binding fragment of an antibody that specifically binds the viral protein optionally wherein the antigen-binding fragment comprises a variable heavy chain (VH) and a variable light chain (VL).

35. The binding agent of claim 34, wherein the antigen-binding fragment is selected from among a Fab fragment, F(ab′)₂ fragment, Fab′ fragment, Fv fragment.

36. The binding agent of any of claims 1-28 and 32-35, wherein the at least one binding domain is an antigen-binding fragment of an antibody selected from among STI-1499, STI-4398, REGN10933, REGN10987, REGN-COV2, JS016, LY-CoV555, LY-3819253, TB181-8, TB181-28, TB181-36, BGB-DXP593, TY027, CT-P59, BRII-196, BRII-198, SCTA01, MW33, AZD8895, AZD1061, HLX70, 15G11, 18F4, 1E5, 1G3, 21C3, 22d(23D11, 26E2, 29F7, 3B3, 3F2, D59047-11955, D70678-12637-S1, D70678-12799-S1, D70678-13531-S1, D70678-13576-S1, D70678-14004-S2, D70678-14027-S2, D70678-2155-S1, D70678-2743-S1, and D70678-5521-S2.

37. The binding agent of any of claims 1-28, 32 and 33, wherein the at least one binding domain is a single domain antibody (sdAb).

38. The binding agent of any of claims 1-28, 32, 33 and 37, wherein the at least one binding domain is TB201-1, TB202-3, TB202-63.

39. The binding agent of any of claims 1-28, 32 and 33, wherein the at least one binding domain is an antibody mimetic, optionally selected from Designed Ankyrin Repeat Protein (DARPin), adnectins, or an antigen-binding fibronectin type III (Fn3) scaffold.

40. A particle, comprising (i) the binding agent of any of claims 1-39 and (ii) a viral protein capable of being bound by the at least one binding domain of the binding agent.

41. The particle of claim 40, wherein the viral protein is a purified viral protein.

42. The particle of claim 40 or claim 41, wherein the viral protein is a recombinant viral protein.

43. The particle of any of claims 40-42, wherein the viral protein is the S (spike) glycoprotein of a coronavirus.

44. A nucleic acid molecule, encoding the binding agent of any of claims 1-39.

45. The nucleic acid molecule of claim 44 wherein the nucleic acid molecule is mRNA.

46. A cell, comprising nucleic acid of claim 44 or claim 45.

47. A vector, comprising the nucleic acid molecule of claim 44 or claim 45.

48. The vector of claim 47, wherein the vector is a viral vector or viral like particle.

49. The vector of claim 47 or claim 48, wherein the vector is derived from an adenovirus.

50. The vector of claim 49, wherein the viral vector is derived from an Adeno-associated virus (AAV).

51. The vector of claim 50, wherein the AAV is of serotype 1, 2, 5, 6, 8 or 9

52. The vector of claim 47 or claim 48, wherein the viral vector is derived from a lentivirus.

53. The vector of any of claims 47-52, wherein the vector comprises a fusogen.

54. The vector of claim 53, wherein the fusogen is a viral fusogen selected from a Class I viral membrane fusion protein, a Class II viral membrane protein, a Class II viral membrane fusion protein, a viral membrane glycoprotein, or a viral envelope protein.

55. The vector of claim 53 or claim 54, wherein the fusogen is a vesicular stomatitis virus envelope glycoprotein (VSV-G), a baboon endogenous virus (BaEV) envelope glycoprotein a syncytin, is a fusogen from a coronavirus, or is derived from a paramyxovirus.

56. The vector of claim 54, wherein the fusogen is a Severe Acute Respiratory Syndrome (SARS) coronavirus 1 (SARS CoV-1) spike glycoprotein, or a Severe Acute Respiratory Syndrome (SARS) coronavirus 2 (SARS CoV-2) spike glycoprotein, optionally wherein the fusogen is an alpha coronavirus CD13 protein.

57. The vector of claim 56, wherein the fusogen derived from a Paramyxovirus comprises an F protein molecule or a biologically active portion thereof and/or a glycoprotein G (G protein) or a biologically active portion thereof.

58. The vector of any one of claims 53-57, wherein the fusogen is a re-targeted fusogen comprising a targeting moiety that binds to a molecule on a target cell.

59. The vector of claim 58, wherein the targeting moiety is a Design ankyrin repeat proteins (DARPin), a single domain antibody (sdAb), a single chain variable fragment (scFv), or an antigen-binding fibronectin type III (Fn3) scaffold.

60. The vector of claim 58 or claim 59, wherein the target cell is known or suspected of being infected by a coronavirus.

61. The vector of any of claims 59-60, wherein targeting moiety binds a receptor of a coronavirus.

62. The vector of any of claims 58-61, wherein the targeting moiety binds angiotensin-converting enzyme 2 (ACE2).

63. The vector of claim 58 or claim 59, wherein the target cell is a B lymphocyte.

64. The vector of any of claim 63, wherein the targeting moiety binds to human CD20.

65. A method of producing a binding agent, comprising introducing the nucleic acid molecule of claim 44 or claim 45 or vector of any of claims 47-64 into a host cell under conditions to express the binding agent in the cell, optionally further comprising isolating or purifying the binding agent from the cell.

66. A method of forming an immune complex, the method comprising administering the one or more binding agent of any of claims 1-39 to a subject known or suspected of having a viral infection, wherein an immune complex comprising the administered one or more binding agent is formed in the subject.

67. The method of claim 66, wherein the immune complex further comprises at least one endogenous antibody against a viral protein exposed on the surface of the virus.

68. The method of any of claims 66 or 67, wherein the one or more binding agent and the at least one endogenous antibody bind the same viral protein.

69. The method of any of claims 66-68, wherein the one or more binding agent and the at least one endogenous agent bind the same epitope of the viral protein, bind a distinct epitope of the viral protein, or bind an overlapping epitope of the viral protein.

70. A composition comprising an immune complex comprising: (i) the binding agent of any of claims 1-39 and (ii) a surface exposed viral protein bound by the at least one binding domain of the binding agent.

71. The composition of claim 70, wherein the immune complex comprises two or more binding agents.

72. The composition of claim 71, wherein the at least two binding domains bind a distinct epitope of the viral protein or bind the same epitope of the viral protein.

73. The composition of claims 70-72, wherein the immune complex further comprises endogenous binding domains, optionally from an endogenous antibody and/or antibodies, and wherein the at least one binding domain and endogenous binding domains bind a distinct epitope of the viral protein or bind an overlapping epitope of the viral protein.

74. A pharmaceutical composition, comprising the binding agent of any of claims 1-39, the nucleic acid of claim 44 or claim 45, or the particle of any of claims 40-43, the cell of claim 46, or the vector of any of claims 47-64.

75. A pharmaceutical composition, comprising (i) the binding agent of any of claims 1-39 and (ii) a recombinant viral protein capable of being bound by the at least one binding domain of the binding agent.

76. The pharmaceutical composition of claim 74 and claim 75, comprising a pharmaceutically acceptable excipient.

77. A method of reducing inflammation in response to a viral infection in a subject, the method comprising administering, to a subject known or suspected of having a virus infection, a therapeutically effective amount of a binding agent of any one of claims 1-39, the particle of any of claims 40-42, the nucleic acid of claim 43 or claim 44, the cell of claim 45, the pharmaceutical composition of claims 74-76, or the vector of any of claims 46-73.

78. A method of reducing inflammation in response to a viral infection in a subject, the method comprising administering to a subject known or suspected of having a virus infection, (i) a therapeutically effective amount of the pharmaceutical composition of claims 74-76; and (ii) a recombinant viral protein capable of being bound by the at least one binding domain of the binding agent.

79. The method of claim 77 or claim 78, wherein the inflammation comprises lymphocytic accumulation in the lung, lymphocytic proliferation in the lung, peripheral blood lymphopenia, pro-inflammatory cytokine production, or combinations of any of the foregoing, optionally wherein the pro-inflammatory cytokine is selected from the group consisting of: MCP-1, IL-8, IL-1β, IFN-γ, IP-10, IL-4, IL-1β, IL-2, IL-7, GCSF, MIP-1A, and TNF-α.

80. The method of claim 78, wherein the pharmaceutical composition and recombinant viral protein are administered concurrently; or wherein the pharmaceutical composition and recombinant viral protein are administered sequentially, optionally wherein the recombinant viral protein is first administered or wherein the pharmaceutical composition is first administered.

81. A method of promoting inhibitory immune complex function, comprising administering therapeutically effective amount of the binding agent of any one of claims 13-39, the particle of any of claims 40-42, the nucleic acid of claim 43 or claim 44, the cell of claim 45, the pharmaceutical composition of claims 74-76, the vector of any of claims 46-73.

82. The method of claim 81, wherein inhibitory immune complex function comprises diminished antigen uptake, diminished antigen presentation, reduced cellular activation, reduced antibody secretion, production of anti-inflammatory cytokines, or combinations of any of the foregoing.

83. A method of reducing activating immune complex function, comprising administering a therapeutically effective amount of the binding agent of any one of claims 1-12, the particle of any of claims 40-42, the nucleic acid of claim 43 or claim 44, the cell of claim 45, the pharmaceutical composition of claims 74-76, or the vector of any of claims 46-73.

84. The method of claim 83, wherein activating immune complex function comprises antibody-dependent cell mediated cytotoxicity (ADCC), antibody dependent enhancement (ADE), release of inflammatory mediators, production of pro-cytokines, phagocytosis, or combinations of any of the foregoing.

85. The method of any of claims 77-84, wherein the method further comprises treatment of a viral infection in a subject.

86. The method of claim 85, wherein the viral infection is an infection caused by a virus with a surface exposed viral protein recognized by the at least one binding domain of the binding agent, optionally wherein the virus is an RNA virus, optionally wherein the RNA virus is selected from SARS-CoV-1, SARS-CoV-2, MERS, RSV, influenza viruses, and measles virus.

87. Use of a binding agent of any one of claims 1-39, the particle of any of claims 40-42, the nucleic acid of claim 43 or claim 44, the cell of claim 45, the pharmaceutical composition of claims 74-76, or the vector of any of claims 46-73, in of the manufacture of a medicament for reducing inflammation in response to a viral infection in a subject.

88. Use of the pharmaceutical composition of claims 74-76, and a recombinant viral protein capable of being bound by the at least one binding domain of the binding agent, in the manufacture of a medicament for reducing inflammation in response to a viral infection in a subject.

89. Use of a binding agent of any one of claims 1-39, the particle of any of claims 40-42, the nucleic acid of claim 43 or claim 44, the cell of claim 45, the pharmaceutical composition of claims 74-76, or the vector of any of claims 46-73, in of the manufacture of a medicament for promoting inhibitor immune complex function.

90. Use of a binding agent of any one of claims 1-39, the particle of any of claims 40-42, the nucleic acid of claim 43 or claim 44, the cell of claim 45, the pharmaceutical composition of claims 74-76, or the vector of any of claims 46-73, in the manufacture of a medicament for reducing activating immune complex function.

91. A binding agent of any one of claims 1-39, the particle of any of claims 40-42, the nucleic acid of claim 43 or claim 44, the cell of claim 45, the pharmaceutical composition of claims 74-76, or the vector of any of claims 46-73, for use in a method of reducing inflammation in response to a viral infection in a subject.

92. A pharmaceutical composition of claims 74-76, and a recombinant viral protein capable of being bound by the at least one binding domain of the binding agent, for use in reducing inflammation in response to a viral infection in a subject.

93. A binding agent of any one of claims 1-39, the particle of any of claims 40-42, the nucleic acid of claim 43 or claim 44, the cell of claim 45, the pharmaceutical composition of claims 74-76, or the vector of any of claims 46-73, for use in a method for promoting inhibitor immune complex function.

94. A binding agent of any one of claims 1-39, the particle of any of claims 40-42, the nucleic acid of claim 43 or claim 44, the cell of claim 45, the pharmaceutical composition of claims 74-76, or the vector of any of claims 46-73, for use in a method for reducing activating immune complex function.


SEQUENCE LISTING (96 sequences)

SEQ ID NO: 1 - protein, 232 aa, Artificial Sequence
  WT IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 235 aa, Artificial Sequence
  WT IgG2 Fc
TKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 229 aa, Artificial Sequence
  WT IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 6 aa, Artificial Sequence
  GS Linker
  REPEAT (1)...(6): 1 to 4 times
  VARIANT 2, 3, 4, 5: Gly or absent
  Xaa is Gly
GXXXXS

SEQ ID NO: 5 - protein, 602 aa, Artificial Sequence
  NiVG protein attachment glycoprotein
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 6 - protein, 604 aa, Artificial Sequence
  Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 7 - protein, 603 aa, Artificial Sequence
  Hendra Virus G Protein without Met
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 8 - protein, 602 aa, Artificial Sequence
  Nipah Virus G Protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 9 - protein, 601 aa, Artificial Sequence
  Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 10 - protein, 622 aa, Artificial Sequence
  Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 11 - protein, 621 aa, Artificial Sequence
  Cedar Virus G Protein (No Met)
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 12 - protein, 632 aa, Artificial Sequence
  Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 13 - protein, 631 aa, Artificial Sequence
  Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 14 - protein, 625 aa, Artificial Sequence
  Bat Paramyxovirus G Protein (No Met) Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 15 - protein, 624 aa, Artificial Sequence
  Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 16 - protein, 546 aa, Artificial Sequence
  Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 17 - protein, 520 aa, Artificial Sequence
  Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 18 - protein, 546 aa, Artificial Sequence
  Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 19 - protein, 520 aa, Artificial Sequence
  Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 20 - protein, 557 aa, Artificial Sequence
  Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 21 - protein, 551 aa, Artificial Sequence
  Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 22 - protein, 545 aa, Artificial Sequence
  Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 23 - protein, 519 aa, Artificial Sequence
  Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 24 - protein, 662 aa, Artificial Sequence
  Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 25 - protein, 640 aa, Artificial Sequence
  Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 26 - protein, 26 aa, Artificial Sequence
  signal sequence
MVVILDKRCYCNLLILILMISECSVG

SEQ ID NO: 27 - protein, 83 aa, Artificial Sequence
  Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 28 - protein, 437 aa, Artificial Sequence
  Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 29 - protein, 597 aa, Artificial Sequence
  NiVG protein attachment glycoprotein Truncated 5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 30 - protein, 582 aa, Artificial Sequence
  NiVG protein attachment glycoprotein Truncated 20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 31 - protein, 577 aa, Artificial Sequence
  NiVG protein attachment glycoprotein Truncated 25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 32 - protein, 498 aa, Artificial Sequence
  Nipah virus NiV-F F0 T234 truncation (aa 525-544) AND mutation on N-linked glycosylation site
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 33 - protein, 524 aa, Artificial Sequence
  Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 34 - protein, 569 aa, Artificial Sequence
  NiVG protein attachment glycoprotein Truncated and mutated (E501 A, W504A, Q530A, E533A) NiV G protein (Gc 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 35 - protein, 568 aa, Artificial Sequence
  NiVG protein attachment glycoprotein Truncated and mutated (E501 A, W504A, Q530A, E533A) NiV G protein (Gc 34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 36 - protein, 524 aa, Artificial Sequence; identical to SEQ ID NO: 33
  Truncated NiV fusion glycoprotein (FcDelta22) at cytoplasmic tail (with signal sequence)

SEQ ID NO: 37 - protein, 500 aa, Artificial Sequence
  Nipah virus NiV-F F0 T234 truncation (aa 525-546)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 38 - protein, 498 aa, Artificial Sequence
  Truncated  mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 39 - protein, 552 aa, Artificial Sequence
  NivG protein attachment glycoprotein Without cytoplasmic tail
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 40 - protein, 604 aa, Artificial Sequence; identical to SEQ ID NO: 6
  Hendra virus G protein

SEQ ID NO: 41 - protein, 603 aa, Artificial Sequence; identical to SEQ ID NO: 7
  Hendra virus G protein Without N-terminal methionine

SEQ ID NO: 42 - protein, 555 aa, Artificial Sequence
  Hendra virus G protein Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 43 - protein, 1260 aa, Artificial Sequence
  SARS CoV 2 S(0), A0A679G9E9
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 602 aa, Artificial Sequence; identical to SEQ ID NO: 5
  NiVG protein attachment glycoprotein

SEQ ID NO: 45 - protein, 1261 aa, Artificial Sequence
  SARS CoV 2 S(0), P0DTC2
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 540 aa, Artificial Sequence
  Sendai F protein
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR

SEQ ID NO: 47 - protein, 575 aa, Artificial Sequence
  Sendai HN protein
MDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 48 - protein, 618 aa, Artificial Sequence
  Sendai HN protein (modified CTD)
MWSELKIRSNDGGEGPEDANDPRGKGVQHIHIQPSLPVGQRVRMDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 49 - protein, 214 aa, Artificial Sequence
  REGN10933 Fab light chain
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 226 aa, Artificial Sequence
  REGN10933 Fab heavy chain
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 51 - protein, 216 aa, Artificial Sequence
  REGN10987 Fab light chain
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - protein, 225 aa, Artificial Sequence
  REGN10987 Fab heavy chain
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 53 - protein, 217 aa, Artificial Sequence
  JS016 light chain
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 54 - protein, 233 aa, Artificial Sequence
  JS016 heavy chain
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTHHHHHH

SEQ ID NO: 55 - protein, 121 aa, Artificial Sequence
  15G11 VDJ
QVQLQQSGAELVRPGVSVKISCKGSGYTFTDYAMHWVKQSHAKSLEWIGVISTYYGDASYNQKFKGKATMTVDKSSSTAYMELARLTSEDSAIYYCARWANWGYYYAMDYWGQGTSVTVSS

SEQ ID NO: 56 - protein, 109 aa, Artificial Sequence
  15G11 VJ
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 57 - protein, 121 aa, Artificial Sequence
  18F4 VDJ
QVQLKESGPGLVAPSQSLSITCTVSGFSLTSYAISWVRQPPGKGLEWLGVIWTGGGTNYNSALKSRLSISKDNSKSQVFLKMNSLQTDDTARYYCARKDYYGSSRNAMDYWGQGTSVTVSS

SEQ ID NO: 58 - protein, 109 aa, Artificial Sequence; identical to SEQ ID NO: 56
  18F4 VJ

SEQ ID NO: 59 - protein, 120 aa, Artificial Sequence
  1E5 VDJ
QVQLQQPGAELVKPGASVKMSCKASGYTFTSYWITWVKQRPGQGLEWIGDIYPGSGSTNYNEKFKSKATLTVDTSSSTAYMQLSSLTSEDSAVYYCARSTVVATDAMDYWGQGTSVTVSS

SEQ ID NO: 60 - protein, 112 aa, Artificial Sequence
  1E5 VJ
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIK

SEQ ID NO: 61 - protein, 121 aa, Artificial Sequence
  1G3 VDJ
QVQLKESGPGLVAPSQSLSITCTVSGFSLTSYAISWVRQPPGKGLEWLGVIWTGGGTNYNSALKSRLSISKDNSKSQVFLKMNSLQTDDTARYYCARFHYYGSSYGYFDYWGQGTTLTVSS

SEQ ID NO: 62 - protein, 109 aa, Artificial Sequence; identical to SEQ ID NO: 56
  1G3 VJ

SEQ ID NO: 63 - protein, 119 aa, Artificial Sequence
  21C3 VDJ
QVQLKESGPGLVAPSQSLSITCTVSGFSLTSYAISWVRQPPGKGLEWLGVIWTGGGTNYNSALKSRLSISKDNSKSQVFLKMNSLQTDDTARYYCARIYYYGSSYFDYWGQGTTLTVSS

SEQ ID NO: 64 - protein, 109 aa, Artificial Sequence
  21C3 VJ
QAVVTQESALTTSPGETVTLTCRSSTGAVTTSNYANWVQEKPDHLFTGLIGGTNNRAPGVPARFSGSLIGDKAALTITGAQTEDEAIYFCALWYSNQFIFGSGTKVTVL

SEQ ID NO: 65 - protein, 118 aa, Artificial Sequence
  22D9 VDJ
QVQLQQSGAELVRPGTSVKVSCKASGYAFTNYLIEWVKQRPGQGLEWIGVINPGSGGTNYNEKFKGKATLTADKSSSTAYMQLSSLTSEDSAVYFCARRHYYYGVDYWGQGTTLTVSS

SEQ ID NO: 66 - protein, 106 aa, Artificial Sequence
  22D9 VJ
DIKMTQSPSSMYASLGERVTITCKASQDINSYLSWFQQKPGKSPKTLIYRANRLVDGVPSRFSGSGSGQDYSLTISSLEYEDMGIYYCLQYDELYTFGGGTKLEIK

SEQ ID NO: 67 - protein, 120 aa, Artificial Sequence
  23D11 VDJ
EVQLQQSGPELVKPGASVKISCKTSGYTFTEYTMYWVKQSHGKSLEWIGGVNPNNGDTSYSQKFKGKATLTVDKSSSTAYMELRSLTSEDSAVYYCARDGYDLYYGMDYWGQGTSVTVSS

SEQ ID NO: 68 - protein, 107 aa, Artificial Sequence
  23D11 VJ
DIVMTQSQKFMSTSVGDRVSVTCKASQNVGTNVAWYQQKPGQSPKALIYSASYRYSGVPDRFTGSGSGTDFTLTISNVQSVDLAEYFCHQYNSYPWTFGGGTKLEIR

SEQ ID NO: 69 - protein, 121 aa, Artificial Sequence
  26E2 VDJ
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTKGYYGSSYDYFDYWGQGTTLTVSS

SEQ ID NO: 70 - protein, 106 aa, Artificial Sequence
  26E2 VJ
DIQMTQSPSSLSASLGGKVTITCKASQDINKYIAWYQHKPGKGPRLLIHYTSTLQPGIPSRFSGSGSGRDYSFSISNLEPEDIATYYCLQYDNLWTFGGGTKLEIK

SEQ ID NO: 71 - protein, 117 aa, Artificial Sequence
  29F7 VDJ
QVQLQQSGPELVKPGASVKISCKASGYAFSSSWMNWVKQRPGKGLEWIGRIYPGDGDTNYNGKFKGKATLTADKSSSTAYMQLSSLTSEDSAVYFCARDDYDEGDYWGQGTTLTVSS

SEQ ID NO: 72 - protein, 106 aa, Artificial Sequence; identical to SEQ ID NO: 70
  29F7 VJ

SEQ ID NO: 73 - protein, 120 aa, Artificial Sequence
  3B3 VDJ
DVKLVESGEGLVKPGGSLKLSCAASGFTFSSYAMSWVRQTPEKRLEWVAYISSGGDYIYYADTVKGRFTISRDNARNTLYLQMSSLKSEDTAMYYCTRVARLYDGYFDYWGQGTTLTVSS

SEQ ID NO: 74 - protein, 111 aa, Artificial Sequence
  3B3 VJ
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPYTFGGGTKLEIK

SEQ ID NO: 75 - protein, 120 aa, Artificial Sequence
  3F2 VDJ
EVQLQQSGAELVRPGASVKLSCTASGFNIKDDYMHWVKQRPEQGLEWIGWIDPENGDTEYASKFQGKATITVDISSNTAYLQLNSLTSEDTAVYYCSTLIYYYGSSNDYWGQGTTLTVSS

SEQ ID NO: 76 - protein, 109 aa, Artificial Sequence; identical to SEQ ID NO: 56
  3F2 VJ

SEQ ID NO: 77 - protein, 119 aa, Artificial Sequence; identical to SEQ ID NO: 63
  D59047-11955 VDJ

SEQ ID NO: 78 - protein, 109 aa, Artificial Sequence; identical to SEQ ID NO: 64
  D59047-11955 VJ

SEQ ID NO: 79 - protein, 118 aa, Artificial Sequence
  D70678-12637-S1 VDJ
EVQLVESGGGLVKPGGSLRLSCATSGFTFSHYSMNWVRQAPGKGLEWVSSISSSSSNIYYADSVKGRFTVSRDNAKNSLYLQMNSLRAEDTAVYYCARRGSSWSFDYWGQGTLVTVSS

SEQ ID NO: 80 - protein, 107 aa, Artificial Sequence
  D70678-12637-S1 VJ
EIVLTQSPATLSLSPGERAILSCRASQSISSTYLAWNQQKPGQAPRLLIYGASSRATGIPDRFSGSGSGTDFTLTISRLEPEDFAVYYCQQYGSSWTFGQGTKVEIK

SEQ ID NO: 81 - protein, 112 aa, Artificial Sequence
  D70678-12799-S1 VDJ
QVQLVQSGAEVKKPGASVKVSCKASGYTFTDYYMHWVRQAPGQGLEWMGWINPNSGGTNYAQKFQGRVTMTRDTSISTAYMELSRLRSDDTALYYCARLDYWSQGTLVTVSS

SEQ ID NO: 82 - protein, 112 aa, Artificial Sequence
  D70678-12799-S1 VJ
DIVMTQTPLSLPVTLGQPASISCRSSQSLVHSDGNTYLSWLQQRPGQPPRLLIYKISNRFSGVPDRFSGSGAGTDFTLKISRVEAEDVGVYYCMQETQFTWTFGQGTKVEIK

SEQ ID NO: 83 - protein, 113 aa, Artificial Sequence
  D70678-13531-S1 VDJ
QVQLQESGPGLVKPSETLSLTCTVSGGSISSYYWTWIRQSPGKGLEWIGYIYYSGSTYTNPSLKSRVTFSVDTSENQFSLKLNSVTAADTAIYFCARDNMDVWGKGTTVTVSS

SEQ ID NO: 84 - protein, 112 aa, Artificial Sequence
  D70678-13531-S1 VJ
DVVMTQTPLSLPVTLGQPASISCRCSQSLVYSDGNTYLNWFQQRPGQSPRRLIYKVSIRDSGVPDRFSGSGSGTDFTLKISRVEAEDVGIYYCMQGTHRPITFGRGTRLEIK

SEQ ID NO: 85 - protein, 113 aa, Artificial Sequence
  D70678-13576-S1 VDJ
QVQLQESGPGLVKPSETLSLTCTVSGGSISNYYWTWIRQPPGKGLEWIGYIYYSGSTYTNPSLKSRVTISVDTSENQFSLKLNSVTAADTAIYYCARDNMDVWGKGTTVTVSS

SEQ ID NO: 86 - protein, 113 aa, Artificial Sequence
  D70678-13576-S1 VJ
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 87 - protein, 123 aa, Artificial Sequence
  D70678-14004-S2 VDJ
QVQLVQSGAEVKKPGASVKVSCKASGYTFSSYGIIWVRQAPGQGLEWMGWISAYNGNTNYAQKLQGRVTMTTDTSTSTAYMELRTLRSDDTAVYYCAREITLNWNYAGWFDPWGQGTLVTVSS

SEQ ID NO: 88 - protein, 108 aa, Artificial Sequence
  D70678-14004-S2 VJ
EIVMTQSPATLSVSPGERATLSCRASQSIRNNLAWYQQKPGQAPRLLIYGAISRATGVPARFSGSGSGTEFTLTISSLQSEDFAVYHCQQYNNWLPYTFGQGTKLEIK

SEQ ID NO: 89 - protein, 117 aa, Artificial Sequence
  D70678-14027-S2 VDJ
EVQLVESGGGLVKPGGSLRLSCAASGFTFSYAWMTWVRQAPGKGLEWVGRIKTKSDGGTTDYASPVKGKFTISRDDSKNTLYLQMNSLQTEDTAVYYCTTHSSPDYWGQGTLVTVSS

SEQ ID NO: 90 - protein, 107 aa, Artificial Sequence
  D70678-14027-S2 VJ
DIQMTQSPSSLSASVGDRVTITCQASQDIRNYLNWYHQKPGKAPKLLIYDASNLETGVPSRFSGSGSGTDFTFTISSLQPEDIATYYCQQYDNLPYTFGQGTKLEIK

SEQ ID NO: 91 - protein, 117 aa, Artificial Sequence
  D70678-2155-S1 VDJ
QVQLVESGGDLVKPGGSLRLSCAASGFTFSNAWMTWVRQAPGKELEWVGRIKTKSDGGTIEYGVSVKGRFTISRDDSKNTLFLQMNSLTTEDTAVYYCTTHSSPDYWGQGTLVTVSS

SEQ ID NO: 92 - protein, 107 aa, Artificial Sequence
  D70678-2155-S1 VJ
DIQMTQSPSSLSASVGDRVTITCQASQDIRNYLNWYQQKPGKAPKLLIYDASTLETGVPSRFSGSGSGTDFTFTISSLQPEDIATYYCHQYGNLPLSFGGGTKVEIK

SEQ ID NO: 93 - protein, 113 aa, Artificial Sequence
  D70678-2743-S1 VDJ
QVQLQESGPGLVKPSETLSLTCTVSGGSISSYYWTWIRQPPGKGLEWIGYIYSSGSTYTNPSLKSRVTISVDTSENQFSLKLNSVTAADTAVYYCARDNMDVWGKGTTVTVSS

SEQ ID NO: 94 - protein, 112 aa, Artificial Sequence
  D70678-2743-S1 VJ
DVVMTQTPLSLPVTLGQPASISCRSSQSLVYSDGNTYLNWFQQRPGQSPRRLIYKVSNRDSGVPDRFSGSGSGTDFTLKISRVEAEDVGIYYCMQGTHWPIIFGRGTRLEIK

SEQ ID NO: 95 - protein, 117 aa, Artificial Sequence
  D70678-5521-S2 VDJ
EVQLVESGGGLVKPGGSLRLSCAASGFTFNYAWMTWVRQAPGKGLDWVGRIKTKTDSGTTDYAAPVKGRFTISRDDSKNTLYLQMNNLKTEDTAVYYCTTHSTPDYWGQGTLVTVSS

SEQ ID NO: 96 - protein, 107 aa, Artificial Sequence
  D70678-5521-S2 VJ
DIQMTQSPSSLSASVGDRVTITCQASQDINNHLNWYQQKPGKAPNLLIYDASNLETGVPSRFSGSGSGTDFTFTISSLQPEDFATYYCQQYDNLPYTFGQGTKLEIK